(19) United States
(12) Patent Application Publication
(10) Pub. No.: US 2014/0234321 A1
(43) Pub. Date: Aug. 21, 2014

(54) HUNK, A SNF1-RELATED KINASE ESSENTIAL FOR MAMMARY TUMOR METASTASIS

(71) Applicant: The Trustees of The University of Pennsylvania, Philadelphia, PA (US)

(72) Inventors: Lewis A. CHODOSH, West Chester, PA (US); Gerald Wertheim, Swarthmore, PA (US); Thomas W. Yang, Long Island City, NY (US)

(73) Assignee: The Trustees of The University of Pennsylvania, Philadelphia, PA (US)

(21) Appl. No.: 14/175,187

(22) Filed: Feb. 7, 2014

Related U.S. Application Data
(63) Continuation of application No. 11/918,640, filed on Dec. 22, 2008, now abandoned.
(60) Provisional application No. 60/671,655, filed on Apr. 15, 2005.

(51) Int. Cl. C12Q 1/68; C12N 15/113; G01N 33/50; G01N 33/574

(52) U.S. Cl. CPC C12Q 1/6886; G01N 33/57415; C12N 15/1137; G01N 33/5011
USPC 424/139.1; 435/6.14; 435/6.11; 514/44 A; 435/6.12; 506/9; 435/7.4; 435/15; 435/6.13

(57) ABSTRACT

This invention relates generally to a novel serine/threonine protein kinase, specifically to hormonally up-regulated, neu-tumor-associated kinase (HUNK); and to the role of HUNK in tumor metastasis, primary tumor development, and the prediction of tumor behavior.

### CROSS REFERENCE TO RELATED APPLICATIONS

This application is a Continuation application which claims the benefit of U.S. National Phase application Ser. No. 11/918,640, filed Dec. 22, 2008 which claims the benefit of International Application No. PCT/US2005/033960, filed Sep. 22, 2005, which claims the benefit of U.S. Provisional Application No. 60/671,655, filed Apr. 15, 2005, the contents of each of which are incorporated herein in their entireties for all purposes.

### FIELD OF THE INVENTION

This invention relates generally to a novel serine/threonine protein kinase, specifically to hormonally up-regulated, neu-tumor-associated kinase (HUNK); and to the role of HUNK in tumor metastasis, primary tumor development, and the prediction of tumor behavior.

### BACKGROUND

A wealth of epidemiological evidence indicates that ovarian hormones play a crucial role in the etiology of breast cancer (Kelsey et al., Epidemiol. Rev. 15:36-47 (1993)). Specifically, the observations that early menarche, late menopause and postmenopausal hormone replacement therapy are each associated with increased breast cancer risk, whereas early oophorectomy is associated with decreased breast cancer risk, have led to the hypothesis that breast cancer risk is proportional to cumulative estradiol and progesterone exposure (Henderson et al., Cancer Res. 48:246-253 (1988); Pike et al., Epidemiol Rev. 15:17-35 (1993)). As such, elucidating the mechanisms by which hormones contribute to mammary carcinogenesis is a central goal of breast cancer research.

In addition to their roles in the pathogenesis of breast cancer, estradiol and progesterone are the principal steroid hormones responsible for regulating the development of the mammary gland during puberty, pregnancy and lactation (Topper et al., Physiol. Rev. 60:1049-1106 (1980)). For example, estradiol action is required for epithelial proliferation and ductal morphogenesis during puberty, whereas progesterone action is required for ductal arborization and alveolar differentiation during pregnancy (Bocchinfuso et al., J. Mamm. Gland Biol. Neoplasia, 2:323-334 (1997); Humphreys et al., J. Mamm. Gland Biol Neoplasia, 2:343-354 (1997); Topper et al., 1980). The effects of estradiol and progesterone in a given tissue are ultimately determined by the activation and repression of their respective target genes.

Protein kinases represent the largest class of genes known to regulate differentiation, development, and carcinogenesis in eukaryotes. Many protein kinases function as intermediates in signal transduction pathways that control complex processes such as differentiation, development, and carcinogenesis (Birchmeier et al., BioEssays, 15:185-190 (1993); Bolen, Oncogene, 8:2025-2031 (1993); Rawlings et al., Immunol. Rev., 138: 105-1 19 (1993); Bolen, Oncogene, 8:2025-2031 (1993); Rawlings et al., Immunol. Rev., 138:105-1 19 (1994)). Accordingly, studies of protein kinases in a wide range of biological systems have led to a more comprehensive understanding of the regulation of cell growth and differentiation (Bolen, 1993; Fantl et al., Annu. Rev. Biochem., 62:453-81(1993); Hardie, Symp. Soc. Exp. Biol., 44:241-255 (1990)).

Not surprisingly, aberrant expression or mutations in several members of the protein kinase family have been reportedly involved in the pathogenesis of cancer both in humans and in rodent model systems (Cardiff et al., Cancer Surv., 16:97-1 13 (1993); Cooper, Oncogenes. Publ., Jones & Bartlett, Boston, Mass., (1990); DiFiore et al., Cell, 51:1063-1070 (1987); Muller et al., Cell, 54:105-1 15 (1988)). Protein kinases function as molecular switches in signal transduction pathways that regulate cellular processes, such as proliferation and differentiation. In addition, some protein kinases are expressed in a lineage-specific manner, and are therefore useful markers for defining cellular subtypes (Dymecki et al., Science, 247:332-336 (1990); Mischak et al., J. Immunol, 147:3981-3987 (1991); Rawlings et al., 1994; Schnurch et al., Development, 119:957-968 (1993); Siliciano et al., Proc. Natl. Acad. Sci. USA, 89:11194-11198 (1992); Valenzuela et al., Neuron, 15:573-584 (1995)).

A key role played by serine/threonine kinases in regulating diverse cellular processes is exemplified by studies of SNF1-related kinases. The SNF1 family of protein kinases is composed of at least two subfamilies. The first subfamily includes SNF1 and its plant homologues including NPK5, Akin 10, BKIN 12, and Rkin1, as well as the mammalian SNF1 functional homologue, AMPK (Alderson et al., Proc. Natl. Acad. Sci. USA, 88:8602-8605 (1991); Carling et al., J. Biol Chem., 269:1 1442-11448 (1994); LeGuen et al., Gene, 120:249-254 (1992); Muranaka et al., Mol. Cell Biol., 14:2958-2965 (1994)). More recently, additional mammalian SNF1-related kinases have been identified that define a second subfamily. These include C-TAK1/p78 (involved in cell cycle control), MARK1, MARK2/Emk, SNRK (involved in adipocyte differentiation), and Msk (involved in murine cardiac development), as well as the C. elegans kinase, PAR-I (Becker et al., Eur. J. Biochem., 235:736-743 (1996); Drewes et al., Cell, 89:297-308 (1997); Peng et al., Science, 277:1501-1505 (1997); Peng et al., Cell Growth Differ., 9:197-208 (1998); Ruiz et al., Mech. Dev., 48:153-164 (1994)). Less closely related to either subfamily are Wpk4, MeIk, and KIN1, SNF1-related kinases found in wheat, mice, and Schizosaccharomyces pombe, respectively (Heyer et al., Mol. Reprod. Dev., 47:148-156 (1997); Levin et al., Proc. Natl. Acad. Sci. USA, 87:8272-8276 (1990); Sano et al., Proc. Natl. Acad. Sci. USA, 91:2582-2586 (1994)).

SNF1 is composed of a heterotrimeric complex that is activated by glucose starvation and is required for the expression of genes in response to nutritional stress (Carlson et al., Genetics, 98:25-40 (1981); Celenza et al., Mol. Cell. Biol., 9:5045-5054 (1989); Ciriacy, Mol. Gen. Genet., 154:213-220 (1977); Fields et al., Nature, 340:245-246 (1989); Wilson et al., Curr. Biol., 6:1426-1434 (1996); Yang et al., Science, 257:680-682 (1992); Yang et al., EMBO J., 13:5878-5886 (1994); Zimmermann et al., Mol. Gen. Genet., 154:95-103 (1977); Hardie et al., Semin. Cell Biol., 5: 409-416 (1994)). In fact, SNF-1 itself has been found to mediate cell cycle arrest in response to starvation (Thompson-Jaeger et al., Genetics, 129:697-706 (1991)).

Like SNF1, the mammalian SNF1-related kinase, AMPK, is involved in the cellular response to environmental stresses, particularly those that elevate cellular AMP:ATP ratios. Once activated, AMPK functions to decrease energy-requiring anabolic pathways, such as sterol and fatty acid synthesis while up-regulating energy-producing catabolic pathways such as fatty acid oxidation (Moore et al., Eur. J. Biochem., 199:691-697 (1991); Ponticos et al., EMBO J., 17: 1688-1699 (1998)). AMPK complements the SNF1 mutation in yeast and phosphorylates some of the same targets as SNF1 (Hardie, Biochem. Soc. Symp., 64:13-27 (1999); Hardie et al., Biochem. Soc. Trans., 25:1229-1231 (1997); Hardie et al., Biochem. J, 338:717-722 (1999); Woods et al., J. Biol. Chem., 271:10282-10290 (1996)). Like SNF1, AMPK is a heterotrimer composed of a, b, and g subunits that are homologous to the subunits of SNF1 (Hardie, 1999). Thus, AMPK and SNF1 are closely related both functionally and structurally, demonstrating that the regulatory pathways in which they operate have been highly conserved during evolution.

For instance, C-TAK 1/p78 appears to be involved in cell cycle regulation based on its ability to phosphorylate and inactivate Cdc25c (Peng et al., 1997; Peng et al., 1998). Since Cdc25c controls entry into mitosis by activating cdc2, inactivation of Cdc25c by C-TAK1 would be predicted to regulate proliferation negatively. Consistent with this model, C-TAK1/p78 is down-regulated in adenocarcinomas of the pancreas (Parsa, Cancer Res. 48:2265-2272 (1988)).

Perhaps the most compelling evidence that SNF1 kinases are involved in development is the observation that mutations in the C. elegans SNF1-related kinase, PAR-1, result in an inability to establish polarity in the developing embryo (Guo et al., Cell, 81:61 1-620 (1995)). Specifically, par-1 mutations disrupt P granule localization, asymmetric cell divisions, blastomere fates, and mitotic spindle orientation during early embryogenesis.

In an analogous manner, the mammalian PAR-I homologue, MARK2/Emk, is asymmetrically localized in epithelial cells in vertebrates, and expression of a dominant negative form of MARK2 disrupts both cell polarity and epithelial cell-cell contacts (Bohm et al., Curr. Biol, 7:603-606 (1997)). In addition, overexpression of either MARK2 or its close family member MARK1 results in hyperphosphorylation of microtubule-associated proteins, disruption of the microtubule array, and cell death (Drewes et al., 1997). Thus, members of the SNF1 kinase family have been demonstrated to regulate a variety of important cellular processes.

However, despite advances in detection and treatment in light of these findings, breast cancer remains the leading cause of cancer mortality among women worldwide, with over 400,000 deaths annually attributed to the disease (Parkin, et al., CA Cancer J Clin, 55:74-108 (2005)). A major determinant of morbidity and mortality associated with breast cancer is the metastatic spread of the tumor cells to distant sites (Jemal, et al., CA Cancer J Clin, 54:8-29 (2004); Ford et al., Dis Mon, 45:333-405 (1999)). Metastases have classically been thought to arise from rare cells within a primary tumor (Fidler et al., Nat Rev Cancer, 3:453-458 (2003)). Accordingly, identifying molecules that contribute to the metastatic process is essential for determining cancer prognosis and developing more effective cancer therapies.

Recently, experiments utilizing DNA microarrays to analyze gene expression patterns in primary tumors have led to a re-evaluation of the understanding of the metastatic process (Ramaswamy et al., Nat Genet, 33:49-54 (2003); Sorlie et al., PNAS, 98:10869-10874 (2001); Sotiriou et al., PNAS, 100:10393-10398 (2003); van 't Veer et al., Nature, 415:530-536 (2002); van de Vijver et al., N Engl J Med, 347:1999-2009 (2002)). These studies have defined gene expression signatures that identify primary tumors predisposed to metastasize. Such signatures are evident prior to the detection of metastases, suggesting that the propensity for tumors to metastasize may be established early in tumor development (Bernards, et al., Nature, 418:823 (2002)). Accordingly, identifying the genes responsible for establishing metastatic phenotypes in primary tumors is critically important.

In light of these findings, it is clear that prior to the present invention, there was a need to identify and study the role of protein kinases in mammary development and carcinogenesis, as well as provide insight into the regulation of pregnancy-induced changes in the mammary tissue that occur in response to estrogen and progesterone. Further, there is a long-felt need to understand the mechanisms and predictors of cancer metastasis in order to custom-tailor the early diagnosis, prognosis, and/or treatment of a cancer patient to attenuate or prevent metastasis. The present invention meets these needs.

### SUMMARY OF THE INVENTION

The present invention was the product of a systematic study of the role of protein kinases in mammary gland development and carcinogenesis. Based upon examination of defined stages in postnatal mammary development and in a panel of mammary epithelial cell lines derived from distinct transgenic models of breast cancer, the inventors discovered a novel SNF1-related serine/threonine kinase, Hunk (Hormonally Up-regulated, Neu-Tumor-Associated Kinase). The isolation of Hunk resulted from the examination of 1500 cDNA clones generated using a RT-PCR-based screening strategy, which identified 41 protein kinases, including 33 tyrosine kinases and 8 serine/threonine kinases, 3 of which were novel.

As used herein, the notation “HUNK” typically refers to human HUNK protein and/or nucleic acids, and the notation “Hunk” typically refers to mouse Hunk protein and/or nucleic acids. However, it to will be understood that either term, “HUNK” or “Hunk” can be used interchangeably to refer to either human or mouse Hunk protein or nucleic acid, unless otherwise specified in the passage set forth herein. Further, when the origin of the Hunk (eg., human or mouse) is specified in a passage set forth herein, it will be understood that the explicit definition of the origin of the protein or nucleic acid supercedes any typographical notation of the term “HUNK” or “Hunk.”

The present invention provides an isolated a 5.0-kb full-length cDNA clone for Hunk that contains the 714-amino-acid open reading frame encoding Hunk. Analysis of this cDNA reveals that Hunk is most closely related to the SNF1 family of serine/threonine kinases and contains a newly described SNF1 homology domain. Accordingly, antisera specific for Hunk detect an 80-kDa polypeptide with associated phosphotransferase activity.

Hunk is located on distal mouse chromosome 16 in a region of conserved synteny with human chromosome 21q22. During fetal development and in the adult mouse, Hunk mRNA expression is developmentally regulated and tissue-specific. Moreover, in situ hybridization analysis reveals that Hunk expression is restricted to subsets of cells within a variety of organs in the adult mouse, indicating a role for Hunk in murine development.

During postnatal mammary development, Hunk mRNA expression is restricted to a subset of mammary epithelial cells and is temporally regulated with highest levels of expression occurring during early pregnancy. In addition, treatment of mice with 17β-estradiol and progesterone resulted in the rapid and synergistic up-regulation of Hunk expression in a subset of mammary epithelial cells, correlating expression of this kinase with regulation by ovarian hormones. Consistent with the tightly regulated pattern of Hunk expression during pregnancy, mammary glands from transgenic mice engineered to mis-express Hunk in the mammary epithelium manifest temporally distinct defects in epithelial proliferation and differentiation during pregnancy, and fail to undergo normal lobuloalveolar development, suggesting a role for Hunk in affecting the changes in the mammary gland that occur during pregnancy in response to ovarian hormones.

Hunk is expressed in a heterogeneous, epithelial-specific manner throughout postnatal mammary development. This heterogeneous expression pattern is particularly striking in the terminal end bud during puberty and throughout the mammary epithelium during pregnancy. Thus, it is an object of the present invention to provide Hunk as a marker for a particular cellular state or a previously undescribed subtype of mammary epithelial cell.

The steroid hormones 17β-estradiol and progesterone play a central role in the pathogenesis of breast cancer and regulate key phases of mammary gland development. Thus, it is an object of this invention to provide developmental regulatory molecules whose activity is influenced by ovarian hormones, which may also contribute to mammary carcinogenesis.

The HUNK kinase has been shown herein to be down-regulated in the majority of human breast cancers compared to benign breast tissue; however, HUNK is overexpressed in approximately 25% of human breast cancers compared to benign breast tissue. Moreover, the range of HUNK expression from highest to lowest in human breast cancer is approximately 70-fold. In addition to its altered expression in human breast cancer, expression of the HUNK kinase has been shown to be elevated in human ovarian carcinomas when compared to benign tissue, and to be positively correlated with tumor grade. In other words, the higher the tumor grade, the higher the expression of the HUNK kinase. Similarly, expression of the HUNK kinase has been shown to be increased in a subset of human colon carcinomas compared to benign tissue, and to be positively associated with tumor grade. Such a correlation between the genes of the present invention and various cancers has not been previously reported, although it is unclear at this point whether the altered expression of the kinase is a coincidental marker of tumor behavior, or whether the altered expression of the kinase is causally related to the cancer.

The present invention also provides a method of delivering an effective amount of an inhibitor of the Hunk kinase to block the activation of, or decrease the activity of, the kinase in the target cell. In particular, the delivered inhibitor comprises an antisense or anti-Hunk molecule. In at least one embodiment, the kinase is overexpressed in the target cell, as compared with a comparable normal cell of the same type.

In addition, the invention provides a method of treating cancer, hyperproliferative disease or oncogene expression in a patient, wherein the method comprises delivering to a target cell in the patient a therapeutically effective amount of an inhibitor of Hunk. As in the previously described method of delivery, the method of treatment comprises delivering an effective amount of an inhibitor of the Hunk kinase to block the activation of, or decrease the activity of, the kinase in the target cell. In particular, the delivered inhibitor comprises an antisense or anti-Hunk molecule. In at least one embodiment, the kinase is overexpressed in the target cell, as compared with a comparable normal cell of the same type.

The present invention further provides a method of diagnosing a cancer, carcinoma, sarcoma, neoplasm, leukemia, lymphoma or hyperproliferative cell disease or oncogene expression in a patient, wherein the method comprises detecting the presence of and/or measuring Hunk activity or a change therein, as compared with a comparable normal cell of the same type. The method effectively detects and/or measures either the overexpression or under expression of Hunk.

Also provided is a method of rapid screening for a selected compound that modulates the activity of Hunk, comprising: (i) quantifying the expression of the kinase from a target cell; (ii) treating the target cell by administering thereto the selected compound, wherein all other conditions are constant with those in the quantifying step; (iii) quantifying the expression of the kinase from the treated target cell; and (iv) comparing the two quantification measurements to determine the modulation of kinase activity achieved by treatment with the selected compound. The method is applicable to screening for either the presence of kinase, or an underexpression or a measurable decrease in kinase activity, or an overexpression or a measurable increase in kinase activity. It further extends to transformation of the target cell.

Further provided is a method of using Hunk or the nucleotide sequence encoding Hunk as a prognostic tool in a patient to detect the presence of, and/or measure the activity or change of activity of the kinase, as a molecular marker in the patient to predict the behavior of a tumor, cancer, carcinoma, sarcoma, neoplasm, leukemia, lymphoma or hyperproliferative cell disease or oncogene expression in the patient, and applying that detection to predict the appropriate therapy for the patient.

It is particularly preferred that the target cell of the methods of the present invention is human, and that the patient is human.

In addition, the present invention provides a recombinant cell comprising Hunk or HUNK, or a vector or recombinant cell comprising same. Also provided is an antibody specific for the Hunk or HUNK, and homologues, analogs, derivatives or fragments thereof having Hunk activity; as well as an isolated nucleic acid sequence comprising a sequence complementary to all or part of the Hunk or HUNK, and to mutants, derivatives, homologues or fragments thereof encoding a cell having Hunk activity. A preferred complementary sequence comprises antisense activity at a level sufficient to regulate, control, or modulate Hunk activity in a target cell expressing the kinase.

Also included in the present invention is a transgenic cell and/or a transgenic animal comprising Hunk or HUNK, or the nucleic acid encoding same. In another aspect, the invention includes a transgenic animal in which Hunk can be inducibly expressed in the mammary gland. In one embodiment, the invention includes a MMTV-rtTA; TetO-Hunk transgenic animal in which Hunk can be inducibly expressed in the mammary gland. In still another aspect, the invention includes a Hunk knockout animal.

Additional objects, advantages and novel features of the invention will be set forth in part in the description, examples and figures which follow, and in part will become apparent to those skilled in the art on examination of the following, or may be learned by practice of the invention.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings, certain embodiment(s), which are presently preferred. It should be understood, however, that the invention is not limited to the precise arrangements and instrumentalities shown.

FIGS. 1A-1C depict the nucleotide and deduced amino acid sequence of Hunk. The composite nucleic acid sequence and conceptual translation of full-length Hunk cDNA are shown. Nucleotide coordinates are shown on the left. Amino acid coordinates are shown in boldface type on the right. A light shaded box indicates the kinase catalytic domain. Dark shaded boxes denote amino acid motifs characteristic of serine/threonine kinases. The SNF1 homology region, SNH, is denoted by a hatched box. The GC-rich region in the 5′-UTR and the putative polyadenylation sequence in the 3′-UTR are underlined by thin and thick lines, respectively. An asterisk denotes the stop codon. A bracket in the 3′ UTR denotes the poly(T) tract, which differs in length between the two independent cDNA clones (clone E8 is shown).

FIGS. 2A-2C depict the expression, identification, and coding potential of Hunk. FIG. 2A depicts a Northern hybridization analysis of poly(A)⁺ RNA from NAF mammary epithelial cells hybridized with a cDNA probe specific for Hunk. The relative migration of RNA size markers is indicated. FIG. 2B depicts the immunoprecipitation of Hunk. Antisera raised against the amino-terminus of Hunk (α-Hunk IP), or against polypeptides unrelated to Hunk (control IP) were used to immunoprecipitate protein from lysates prepared from cells that either express (+) or do not express (−) Hunk mRNA. Immunoprecipitated protein was immunoblotted with antisera raised against the carboxyl-terminus of Hunk. FIG. 2C depicts an immunoblotting analysis of Hunk protein using antisera raised against the carboxyl-terminus of Hunk. IVT reactions were performed in rabbit reticulocyte lysates in the presence of unlabeled methionine using either plasmid control (vector) or full-length Hunk cDNA (E8) as a template. NT reaction products were resolved by SDS-PAGE along with lysates from Hunk-expressing (+) and non-expressing (−) cell lines. The relative migration of the closest molecular weight marker is indicated.

FIGS. 3A and 3B depict a segregation analysis of Hunk within the distal region of mouse chromosome 16 as determined by interspecific back-cross analysis. The segregation patterns of Hunk and flanking genes in backcross animals that were typed for all loci are shown at the top of the figure, although for individual pairs of loci, more than 104 animals were typed. FIG. 3A graphically shows that the segregation patterns of Hunk and flanking genes in the loci are shown at the top of the figure. Each column of FIG. 3A represents the chromosome identified in the backcross progeny that was inherited from the (C57BL/6J×M. spretus) F1 parent The shaded boxes in FIG. 3A represent the presence of a C57BL/6J allele, and white boxes represent the presence of a M. spretus allele. The number of offspring inheriting each type of chromosome is listed at the bottom of each column in FIG. 3A. A partial chromosome 16 linkage map showing the location of Hunk in relation to linked genes is shown in FIG. 3B. Recombination distances between loci in centimorgans are shown to the left of the chromosome, and the positions of loci in human chromosomes, where known, are shown to the right. References for the human map positions of loci cited from GDB (Genome Data Base).

FIGS. 4A and 4B depict the kinase activity associated with the Hunk gene product. FIG. 4A depicts immunoblotting using amino-terminal anti-Hunk antisera to analyze Hunk protein expression. Protein extracts are from MMTV-Hunk transgenic (TG) or wildtype (WT) mice, or HC11 cells, a mammary epithelial cell line that does not express Hunk mRNA (−). The relative migration of the 78-kDa marker is indicated. FIG. 4B depicts an in vitro kinase assay of Hunk immunoprecipitates. An arrowhead indicates the relative migration of histone H⁺, used as an in vitro kinase substrate.

FIGS. 5A-5K depict expression of Hunk during murine embryogenesis. FIG. 5A depicts Northern hybridization analysis of poly(A)⁺ RNA from day E6.5, E13.5, and E 18.5 embryos hybridized with a cDNA probe specific for Hunk. The 28S ribosomal RNA band is shown as a loading control. FIGS. 5B-5K depict in situ hybridization analysis of Hunk mRNA expression. FIGS. 5D, 5F, 5G and 5H depict bright-field, and FIGS. 5B, 5C, 5E, 5I, 5J and 5K) depict dark field photomicrographs of E13.5 (FIG. 5B) and E18.5 (FIGS. 5C-5K) FVB embryo sections hybridized with a ³⁵S-lableled Hunk antisense cDNA probe. Tissues shown are kidney (FIGS. 5D, 5E), whisker hair follicles (FIGS. 5F, 5I), submandibular gland (FIGS. 5G, 5J), and skin (FIGS. 5H, 5K). No signal over background was detected in serial sections hybridized with sense Hunk probes: bo, bowel; fv, fourth ventricle; ki, kidney; li, liver; lu, lung; lv, lateral ventricle; oe, olfactory epithelium; sg, submandibular gland; sk, skin; st, stomach; wf, whisker hair follicle. Magnification: 8× (FIGS. 5B, 5C); 20× (FIGS. 5D-5K). Exposure times were optimized for each panel.

FIGS. 6A-6M depict tissue-specific expression of Hunk in adult tissues. FIG. 6A depicts RNase protection analysis of Hunk mRNA expression in tissues of the adult mouse hybridized with antisense RNA probes specific for Hunk and for β-actin. FIGS. 6B-6M depict spatial localization of Hunk expression in tissues of the adult mouse. FIGS. 6B, 6D, 6F, 6H, and 6J depict bright field, and FIGS. 6C, 6E, 6G, 6I, 6K, 6L and 6M depict dark field photomicrographs of in situ hybridization analysis performed on sections of duodenum (FIGS. 6B, 6C), uterus (FIGS. 6D, 6E), prostate (FIGS. 6F, 6G), ovary (FIGS. 6H, 6I), thymus (FIGS. 6J-6L), and brain (FIG. 6M), hybridized with a ³⁵S-labeled Hunk antisense probe. No signal over background was detected in serial sections hybridized with a sense Hunk probe. Arrows indicate cells expressing Hunk at high levels. CA1 and CA3, regions of the hippocampus; cl, corpus luteum; co, cortex; d, epithelial duct; cis, dentate gyrus; g, endometrial gland; fo, follicle; ic, intestinal crypt; me, medulla; mg, mesometrial gland; mu, mucosa; pc, PAR1 et al. cortex; se, serosa; st, stroma. Magnification: 10× (FIG. 6M), 90× (FIGS. 6H-6K), 180× (FIGS. 6B, 6C), 300× (FIGS. 6D, 6E) and 500× (FIGS. 6F, 6G, 6L, 6M).

FIGS. 7A-7C depict temporal regulation of Hunk expression during mammary gland development. FIG. 7A depicts RNase protection analysis of Hunk mRNA expression during postnatal developmental of the murine mammary gland. Total RNA isolated from mammary glands at the indicated time points was hybridized to a ³²P-labeled antisense RNA probe specific for Hunk. A ³²P-labeled antisense RNA probe specific for β-actin was included in the same hybridization reaction as an internal loading control. FIG. 7B depicts phosphorimager analysis of RNase protection analysis described in FIG. 7A. Expression levels are shown relative to adult virgin (15 wk). FIG. 7C depicts in situ hybridization analysis of Hunk expression during pregnancy and lactation. Bright-field (top panel) and dark-field (bottom panel) photomicrographs of mammary gland sections from day 7 pregnant, day 20 pregnant or day 9 lactating animals hybridized with an ³⁵S-labeled Hunk-specific antisense probe. No signal over background was detected in serial sections hybridized with a sense Hunk probe. Exposure times were identical for all dark-field photomicrographs to illustrate changes in Hunk expression during pregnancy, al, alveoli; d, duct; lo, lobule; st, adipose stroma.

FIGS. 8A-8F depict the heterogeneous expression of Hunk in the mammary epithelium as demonstrated by in situ hybridization analysis of Hunk expression in the virgin mammary gland using a ³⁵S-labeled Hunk-specific antisense probe. FIGS. 8A-8C depict bright field and FIGS. 8D-8F depict dark field photomicrographs of in situ hybridization analysis performed on mammary gland sections from nulliparous females. A heterogeneous expression pattern of Hunk is seen is all cases, in both epithelial ducts (FIGS. 8A, 8C, 8D and 8F), and terminal end buds (FIGS. 8B, 8C, 8E and 8F). No signal over background was detected in serial sections hybridized with a sense Hunk probe. Exposure times were optimized for each dark-field panel, d, duct; eb, terminal end bud.

FIGS. 9A-9D show that ovarian hormones alter Hunk mRNA expression in vivo in mammary glands and uteri of mice. Northern blots depict total RNA expression of tissues (mammary glands, FIG. 9A; or uteri, FIG. 9B), harvested from either intact females (sham) or oophorectomized females that to received daily subcutaneous injections of either PBS carrier alone (OVX), 17β-estradiol (OVX+E₂), progesterone (OVX+P), or both 17β-estradiol and progesterone (OVX+E₂+P). Each sample represents a pool of samples hybridized overnight with ³²P-labeled antisense RNA probes specific for Hunk and β-actin. Signal intensities were quantified by phosphorimager analysis and Hunk expression was normalized to β-actin expression levels. Hunk expression relative to expression in oophorectomized (OVX) controls is shown below each lane. FIG. 9C depicts quantification of Hunk expression in mammary glands and uteri from intact FVB female mice after injection with PBS (control; light shaded boxes) or a combination of 5 mg progesterone in 5% gum arabic; and 20 μg of 17β-estradiol in PBS (+E₂+P; dark shaded boxes). RNase protection analysis was performed on either breast or uterus total RNA using ³²P-labeled antisense RNA probes specific for Hunk and β-actin. Hunk expression was quantified by phosphorimager analysis and normalized to β-actin. Values are shown relative to control animals. Each bar represents the average of 4 animals±s.e.m. for each group. FIG. 9D depicts in situ hybridization analysis of Hunk expression in mammary gland sections from oophorectomized mice treated with hormones as described in FIG. 9A. Dark-field exposure times were identical in all cases, al, alveoli; d, duct; st, adipose stroma.

FIGS. 10A-10E depict MMTV-Hunk transgene expression in MHK3 transgenic mice. FIG. 10A depicts Northern hybridization analysis of MMTV-Hunk transgene expression in mammary glands from 7- to 9-week-old nulliparous wild type or MHK3 transgenic mice using a ³²P-labeled probe specific for Hunk. The detected mRNA transcript corresponds to the expected size of the MMTV-Hunk transgene. FIG. 10B depicts an RNase protection analysis of MMTV-Hunk: transgene expression in organs from a 7-week-old nulliparous MHK3 transgenic female mouse. A ³²P-labeled antisense RNA probe spanning the junction of the 3′ end of the Hunk cDNA and the 5′ end of the SV40 polyadenylation signal was used to specifically detect transgene expression in 20 μg of total RNA. A ³²P-labeled antisense RNA probe for β-actin was used in the same reaction to control for RNA loading and sample processing. FIG. 10C depicts the immunoprecipitation of Hunk protein from lactating MHK3 transgenic animals. Affinity-purified antisera raised against the C-terminus of Hunk (α-Hunk) was incubated with protein extract from mammary glands harvested from either MHK3 transgenic (Tg) or wild type (Wt) mice during lactation. A control reaction was performed without antisera (no Ab). Immunoprecipitated protein was analyzed by immunoblotting using C-terminal anti-Hunk antisera. The expected migration of Hunk is indicated. FIG. 10D depicts an in vitro kinase assay of anti-Hunk immunoprecipitates. Histone H1 was used as an in vitro kinase substrate for protein immunoprecipitated with (+) or without (−) anti-Hunk antisera from extracts containing equal amounts of protein as in FIG. 10C. The relative migration of histone H1 is indicated. FIG. 10E depicts an immunohistochemical analysis of Hunk protein expression in MHK3 transgenic mice. Anti-Hunk antisera from FIG. 10C and FIG. 10D were used to detect Hunk protein in sections from paraffin-embedded mammary glands harvested from 14-week-old nulliparous wild type or MHK3 transgenic females. A control assay was performed by omitting primary antisera from the protocol. Detection reaction times were identical in all cases.

FIGS. 11A and 11B depict the effects of Hunk overexpression on RNA content and mammary epithelial proliferation. FIG. 11A depicts the amount of total RNA isolated from either wild type (light-shaded boxes), expressing MHK3 transgenic (dark-shaded boxes), or non-expressing MHK3 transgenic (hatched boxes) female mice during mammary development. Total RNA was isolated from mammary glands harvested from female mice at the indicated developmental time points. The average total RNA yield for each group is represented as the mean±s.e.m. At least 3 mice were analyzed from each group. There is a significant difference in RNA content between wild type and transgenic mammary glands at day 18.5 of pregnancy, and day 2 of lactation (t-test, P=0.047 and 0.0007, respectively). FIG. 11B depicts the relative percentage of BrdU-positive epithelial cells in the mammary glands of wild type and MHK3 transgenic mice during development (t-test, P=0.004).

FIGS. 12A and 12B depict morphological defects in MHK3 transgenic mice during late pregnancy and lactation. Mammary glands from MHK3 transgenic and wild type females were harvested at day 12.5 and day 18.5 of pregnancy, and day 2 of lactation. At least 3-transgene-expressing mice and 3-wild type mice were analyzed for each time point. A representative photomicrograph is shown for each group. FIG. 12A depicts a whole-mount analysis of transgenic and wild type mammary glands at the indicated time points. FIG. 12B depicts a representative hematoxylin and eosin-stained sections of paraffin-embedded transgenic and wild type mammary glands, al, alveoli; lo, lobule; st, adipose stroma.

FIGS. 13A-13F depict differentiation defects in MHK3 transgenic mice during pregnancy and lactation. FIGS. 13A-13D depict Northern analysis of gene expression for epithelial differentiation markers (β-casein, κ-casein, lactoferrin, WAP, and ε-casein) in the mammary glands of wild type or MHK3 transgene-expressing animals at day 6.5 of pregnancy (FIG. 13A), day 12.5 of pregnancy (FIG. 13B), day 18.5 of pregnancy (FIG. 13C), or at day 2 of lactation (FIG. 13D). Differentiation marker expression in the mammary glands of non-expressing MHK3 transgenic animals is also shown in FIG. 13D. β-actin expression is shown as a control for dilutional effects, and the 28S ribosomal RNA band is shown as a loading control. FIG. 13E summarizes a multivariate regression analysis of expression products shown in FIGS. 13A-13D, demonstrating the effects of transgene expression and developmental stage on the natural logarithm of cytokeratin 18 and expression levels of milk protein genes. All expression levels were normalized to β-actin. The P value for the significance of the regression model was <0.01 for all differentiation markers shown. FIG. 13F graphically depicts phosphorimager quantification of Northern analyses of expression products shown in FIGS. 13A-13D. Expression levels of milk protein genes were normalized to β-actin expression and are shown on a logarithmic scale in arbitrary units relative to expression levels first detected in wild type animals. Values are shown as the mean±s.e.m. for each point. The number of mice analyzed in each group is: 4 Wt, 5 Tg (d6.5); 3 Wt, 3 is Tg (d12.5 and d18.5); and 4 Wt, 4 Tg, 4 non-expressing Tg (d2 Lact).

FIG. 14 graphically depicts up-regulation of lactoferrin expression at specific developmental stages in MHK3 mammary glands. Analysis of differentiation marker expression in mammary glands from either wild type (light-shaded boxes), MHK3 transgene-expressing (dark-shaded boxes), or non-expressing MHK3 transgenic (hatched boxes) female mice during puberty or day 2 of lactation, as described in FIG. 13. Northern hybridization. analysis and quantification was performed on virgin or day 2 lactating mice. Total RNA was isolated from mammary glands using ³²P-labeled cDNA probes specific for milk protein genes, as indicated. Expression of these genes was normalized to that of β-actin. Wild type expression values were set to 1.0 and are represented as the mean±s.e.m. for each group.

FIGS. 15A-15B depicts that HUNK is differentially expressed among human breast cancer cell lines and primary human breast cancers. FIG. 15A is an image depicting RNase protection analysis of HUNK and β-actin expression levels in a panel of actively growing human breast tumor cell lines. FIG. 15B is a histogram depicting relative HUNK expression in a panel of primary human breast cancers and normal human breast samples determined by quantitative real-time RT-PCR analysis. HUNK expression is normalized to TBP. Indicated expression levels are relative to average expression in normal breast tissue (mean expression is defined as 1.0). The range of HUNK expression falling within three standard deviations of the mean in normal breast tissue is indicated.

FIGS. 16A-16E depict that the HUNK-expression signature is associated with human breast cancers of high metastatic potential and predicts clinical outcome. FIG. 16A is a schematic representation of the overlap between genes associated with high HUNK expression in primary breast cancers (List A, 195 genes) and genes associated with human breast cancers of high metastatic potential (List B, 77 genes) as determined by van't Veer et al. (Nature 415:530-536 (2002)). From a pool of 8145 genes that are represented on both array platforms, an overlap of 15 genes was detected (p=2.7×10⁻¹⁰, hypergeometric test). A hierarchical clustering (Ward's method) of human breast cancers analyzed by van't Veer et al. (2002) was based on the HUNK-expression signature. Tumor clusters, designated A-D, were ordered based on similarity to the high HUNK-expression signature (cluster A=least similar, cluster D=most similar). The clustering considered genes overexpressed in HUNK-expressing tumors indicated and genes overexpressed HUNK in non-expressing tumors. FIGS. 16B-16D depict metastasis-free survival curves associated with the cancers analyzed by van't Veer et al. (Nature 415:530-536 (2002)) (FIG. 16C), Sortie et al. (PNAS, 100:8418-8423 (2003)) (FIG. 16C), and Ma et al. (Cancer Cell 5:607-616 (2004)) (FIG. 16D) as clustered by the HUNK-expression signature. FIG. 16E is a graphic depiction of metastasis-free survival of human breast cancers with high HUNK mRNA expression (upper quartile) versus cancers with low HUNK expression (lower three quartiles) as determined by Ma et al. (Cancer Cell 5:607-616 (2004)).

FIGS. 17A-17D illustrate the generation and characterization of Hunk knockout mice. FIG. 17A is a schematic diagram of the mouse Hunk genomic locus, targeting vector, and targeted allele (top). The locations of Hunk exon 1, the neomycin cassette flanked by loxP sites (Neo) and the diphtheria toxin A (DTA) gene are depicted. Locations of probes used in Southern hybridization analyses are indicated. K=Kpn I, N=Not I, Ns=Nsi I, X-Xho I, Xm=Xmn I. Restriction sites that were destroyed in cloning are in parentheses. Southern hybridization analysis of targeted ES cell DNA and DNA from offspring of a Hunk heterozygous cross (bottom). The band corresponding to the wild-type allele is 10.3 kb in size, whereas the band corresponding to the targeted allele is 7.0 kb. FIG. 17B is a Western Blot depicting the immunoprecipitation of Hunk from lung tissue of Hunk wild type (+/+), heterozygous (+/−), and homozygous mutant (−/−) mice. Protein lysates from HC11 cells stably transfected with an empty vector or a Hunk-expression vector were included as controls. FIG. 17C is a graph depicting tumor-free survival curves of MMTV-c-myc-induced mammary tumors in Hunk wild type, heterozygous, and homozygous mutant mice. No statistical difference among the three genotypes was observed (p>0.05 for all comparisons, log rank test). FIG. 17D is a series of images depicting histological analysis of Hunk wild type and Hunk-deficient MMTV-c-myc-induced mammary tumors. No difference in tumor morphology was apparent between Hunk genotypes.

FIGS. 18A-18F illustrate that Hunk-knockout mice display a cell-autonomous defect in metastasis. FIG. 18A is an image depicting the gross and histological appearance of lung metastases arising from MMTV-c-myc-induced tumors. FIG. 18F is an image illustrating that an H&E stained section reveals established metastasis (lower right). FIG. 18B is a graph depicting percentage of mice with grossly visible lung metastases within each Hunk genotype. A significantly lower percentage of mice with tumor metastases is observed for Hunk knockout animals relative to controls (Fisher's exact test). FIG. 18C is a graphic depicting tail vein injection of tumor cells into the circulation of nude mice. No differences in the frequency of metastasis were observed between genotypes. FIG. 18D is a series of images depicting soft agar colony formation assay of Hunk wild type and Hunk knockout MMTV-c-myc-induced tumor cells. No differences in the total number or size of colonies were noted. Error bars represent standard error of the mean. FIG. 18E is a graph depicting orthotopic injection of tumor cells in the fat pads of nude mice. A significantly lower frequency of metastasis was observed for Hunk-deficient mammary tumor cells.

FIG. 19 illustrates that the murine Hunk-expression signature predicts human breast cancer metastasis. Based on a hierarchical clustering of Hunk wild-type and Hunk-deficient mouse mammary tumors, tumors were found to segregate into two clusters that correspond to Hunk genotype. Based the hierarchical clustering (Ward's method) of human breast cancers (van't Veer et al., Nature 415:530-536 (2002)), based on genes differentially expressed in Hunk wild type and Hunk knockout tumors, FIG. 19 is a graphic depiction of metastasis-free survival curves of the four tumor clusters.

FIGS. 20A and 20B depict the alignment of mouse (SEQ ID NO:2) and human (SEQ ID NO:17) Hunk polypeptide sequences. Identical amino acids are illustrated in bold, boxed, and shaded print. Conservative amino acid substitutions are illustrated by boxed and non-shaded print. Amino acids 61 to 320 comprise the kinase domain, and the Snf homology region extends from amino acid residue 340 to amino acid 385.

FIGS. 21A and 21IB are a series of images illustrating that mammary gland development is not perturbed in Hunk-deficient animals. FIG. 21A is a series of images depicting carmine-stained whole mount analyses of Hunk wild type and Hunk-deficient mammary gland development. FIG. 2 IB is a series of images depicting eosin-stained histologic analyses of Hunk wild type and Hunk-deficient murine mammary glands. Mammary glands were harvested from female animals at the developmental states, as indicated in the column on the left side of the figure.

FIG. 22 is a graph depicting the ˜2-fold (25 week) increase in mean tumor latency of Hunk-deficient MMTV-Neu animals as compared to Hunk-wild type MMTV-Neu control animals.

FIG. 23 is a graph depicting that Hunk-deficient animals displayed decreased tumor multiplicity when compared to wild type control animals.

FIGS. 24A and 24B are a graphic representation of a centroid including those genes best able to distinguish Hunk wild-type from Hunk knockout myc-induced tumors.

FIG. 25 is graph depicting a classification of human breast cancer samples from the van't Veer data set, based on the mouse Hunk centroid set forth in FIG. 24. The data were divided into groups including those similar to Hunk wild-type tumors (High Hunk), those similar to Hunk knockout tumors (Low Hunk), and those in an intermediate group (Unclassified). Kaplan-Meier metastasis-free survival curves were then generated for each of these three groups.

FIGS. 26A and 26B are a graphic representation of a human HUNK centroid based on microarray expression profiles from human breast cancers expressing either high levels of HUNK or low levels of HUNK.

FIG. 27 is a graph depicting a classification of human breast cancer samples from the van't Veer data set, based on the mouse Hunk centroid set forth in FIG. 26, divided into those most similar to high HUNK-expressing (High HUNK), low HUNK-expressing (Low HUNK), or intermediate (unclassified) breast cancers. Kaplan-Meier metastasis-free survival curves were then generated for each of these three groups.

FIG. 28 is a graph depicting a classification of human breast cancer samples from the Wang data set, based on the mouse Hunk centroid set forth in FIG. 26, divided into those most similar to high HUNK-expressing (High HUNK), low HUNK-expressing (Low HUNK), or intermediate (unclassified) breast cancers. Kaplan-Meier metastasis-free survival curves were then generated for each of these three groups.

FIG. 29 is a graph depicting a classification of human breast cancer samples from the Sorlie data set, based on the mouse Hunk centroid set forth in FIG. 26, divided into those most similar to high HUNK-expressing (High HUNK), low HUNK-expressing (Low HUNK), or intermediate (unclassified) breast cancers. Kaplan-Meier metastasis-free survival curves were then generated for each of these three groups.

FIG. 30 is a graph depicting a classification of human breast cancer samples from the Ma data set, based on the mouse Hunk centroid set forth in FIG. 26, divided into those most similar to high HUNK-expressing (High HUNK), low HUNK-expressing (Low HUNK), or intermediate (unclassified) breast cancers. Kaplan-Meier metastasis-free survival curves were then generated for each of these three groups.

FIG. 31 is a graph depicting that, utilizing the MMTV-Neu model system, Hunk-knockout MMTV-rtTA/TetOp-NeuNT (MTB/TAN) mice displayed a ˜2-fold increase in tumor latency.

FIGS. 32A and 32B provide an illustration of the effect of Hunk-expression on the appearance of hyperplastic lesions. FIG. 32A is a series of images depicting the difference between Hunk wild type and Hunk deficient mice examined at necropsy, in which it was observed that a number of the glands not bearing bona fide tumors did in fact bear hyperplastic lesions. FIG. 32B is a graph depicting that the incidence of hyperplastic lesions was also decreased in Hunk-deficient, non-tumor bearing mammary glands.

FIGS. 33A and 33B illustrate the differential tissue staining of Hunk-expressing and Hunk-deficient tissue. FIG. 33A is a series of images illustrating that for carmine-stained mammary glands induced for four days with doxycycline, no differences were observed when comparing Hunk-wild type and Hunk-knockout MTB/TAN mammary glands. FIG. 33B is a series of images illustrating that no differences were observed in hematoxylin and eosin stained sections of Hunk-wild type and Hunk-knockout MTB/TAN mammary glands.

FIGS. 34A and 34B illustrate the extent of differences in epithelial cell proliferation among various Hunk genotypes. FIG. 34A is a series of images depicting that no statistically significant differences in epithelial cell proliferation were observed between Hunk genotypes. Imaging was conducted using BrdU incorporation as a surrogate for cellular proliferation, and anti-BrdU immunohistochemistry was preformed on 6 Hunk-wild type and 6 Hunk-knockout MTB/TAN mammary glands induced for 96 hrs. FIG. 34B is a series of images depicting that no statistically significant differences in epithelial cell proliferation were observed between Hunk genotypes. Imaging was conducted using TUNEL staining with 6 Hunk-wild type and 6 Hunk-knockout MTB/TAN mammary glands induced for 96 hrs. No TUNEL positive epithelial cells were observed in either Hunk-wild type or Hunk-knockout mammary glands.

FIGS. 35A-C illustrate differences in tumor growth among MW and MK tumors. FIG. 35A is a graph illustrating that no differences in tumor growth were observed, although animals harboring MK1-derived tumors demonstrated a 3-fold to 6-fold decreased incidence of metastasis when compared to animals harboring MW1- or MW4-derived tumors. FIG. 35B is a graph illustrating that when animals were sacrificed upon reaching a mean tumor cross-sectional area of 225 mm2, 75% (6/8) animals harboring MW1-derived tumors presented metastases, whereas none of the animals harboring MK1-derived tumors exhibit metastases (0/8). FIG. 35C is a series of images illustrating that inspection of lungs from animals harboring MK1-derived tumors by H&E did not yield any evidence of metastasis.

FIGS. 36A and 36B translocation of cells as a function of Hunk expression. FIG. 36A is a graph illustrating that ˜12-fold-fewer MK1 cells migrated across a TRANSWELL chamber membrane when compared to MW1 and MW4 cells. FIG. 36B is a graph illustrating that Hunk-deficient cells were found to translocate less frequently (˜2.4-fold) than their wild-type control counterparts.

FIGS. 37A-D illustrate the effect of mutant Hunk on characteristics of cells harboring the mutant protein. FIG. 37A is an image of an electrophoretic gel illustrating a kinase-dead form of Hunk (Hunk K91M). Hunk K91M bears a lysine to methionine substitution at a conserved residue in subdomain II, which is critical for the ATP-binding pocket. Similar substitutions have been utilized to inactivate other kinases without altering substrate binding. Independent, stably transduced pools of MK1 cells expressed readily detectable levels of both Hunk (MK1 H) and Hunk K91M (MK1K), when compared to empty vector controls (MK1E). FIG. 37B is an image of an electrophoretic gel illustrating that Hunk is K91M transduction was not accompanied by an increase in immunoprecipitated kinase activity (FIG. 37B). These results demonstrate that the K91M substitution results in an inactive Hunk kinase. FIG. 37C is a graph depciting the ability of Hunk to promote cellular migration. Hunk-transduced stable pools were seeded in Biocoat™ control inserts. Hunk expressing MK1 cells consistently translocated ˜2.3 fold more frequently than empty vector controls and ˜2.8 fold more frequently than Hunk K91M expressing pools. FIG. 37D is a graph illustrating that when Hunk-induced stable pools are plated on Matrigel-coated Biocaot™ inserts, Hunk expressing stable pools translocated ˜2.3 fold more frequently than empty vector controls and ˜3.0 fold more frequently than Hunk K91M expressing pools.

FIGS. 38A-D illustrate the effect of Hunk on the growth and behavior of transplanted cells. FIG. 38A is a graph depicting that stably-transduced cell lines orthotopically transplanted into the fat pads of nude mice, where mice were monitored for tumor growth and sacrificed upon reaching a mean tumor cross-sectional area of 225 mm², reveal no differences in tumor growth, consistent with the results set forth herein regarding observations of primary tumors and MW1 and MK1 tumors. FIG. 38B is a series of images illustrating that histological inspection of the tumors by H&E revealed no discernable differences between cohorts. FIG. 38C is a graph illustrating that upon inspection of the lungs, animals harboring tumors derived from Hunk expressing pools displayed a ˜11.6 fold increase in incidence of metastases when compared to empty vector controls and a ˜7.3 fold increase in incidence of metastases when compared to Hunk K91M expressing controls. FIG. 38D is a series of images illustrating the results set forth in FIG. 38C.

### DETAILED DESCRIPTION OF THE INVENTION

The present invention provides a novel SNF1-related serine/threonine kinase, Hunk, in the mammary gland, and methods of use therefor, particularly involving its role in mammary development or carcinogenesis. To better understand the relationship between development and carcinogenesis in the breast, a screen was designed to identify protein kinases that are expressed in the murine mammary gland during development and in mammary tumor cell lines (Chodosh et al., Dev. Biol., 219:259-276 (2000); Gardner et al., Genomics 63:279-288 (2000A); Gardner et al., Genomics 63: 49-59 (2000B); Gardner et al. Development 127:4493-4509 (2000); Stairs et al., Hum. Mol. Genet. 7:2157-2166 (1998), each of which is incorporated herein in its entirety).

After kinases were clustered on the basis of similarities in their temporal expression profiles during mammary development, multiple distinct patterns of expression were observed. Analysis of these patterns revealed an ordered set of expression profiles in which successive waves of kinase expression occur during development. This resulted in the identification of a novel serine/threonine kinase of the present invention, the Hormonally Up-Regulated, Neu-Tumor-Associated Kinase (HUNK). Originally referred to as Bstk1 (before being renamed Hunk), the kinase was first identified as a 207-bp RT-PCR product isolated from a mammary epithelial cell line derived from an adenocarcinoma arising in an MMTV-neu transgenic mouse (Chodosh et al., Cancer Res. 59:S1765-S1771 (1999)).

The cDNA encoding Hunk expression in the mammary gland was subsequently found to be: (i) tightly regulated during mammary development with a transient peak during early pregnancy; (ii) rapidly and synergistically induced in response to steroid hormones (17β-estradiol and progesterone); (iii) spatially restricted within a subset of mammary epithelial cells throughout postnatal development; and (iv) preferentially expressed in mammary tumor cell lines derived from MMTV-neu, but not in MMTV-c-myc transgenic mice, leading to the choice of the name for the kinase. These data suggest a role for Hunk in mammary development, particularly with respect to pregnancy-induced changes in the mammary gland.

Consistent with this hypothesis, mis-expression of Hunk in the mammary gland disrupted normal lobuloalveolar development during pregnancy and lactation. Specifically, dysregulated Hunk expression resulted in decreased epithelial cell proliferation exclusively during mid-pregnancy, as well as impaired alveolar cell differentiation throughout pregnancy and lactation. Together, these data show that Hunk plays a role in pregnancy-induced changes in the mammary gland, and that Hunk may be involved in the response of the mammary epithelium to ovarian hormones.

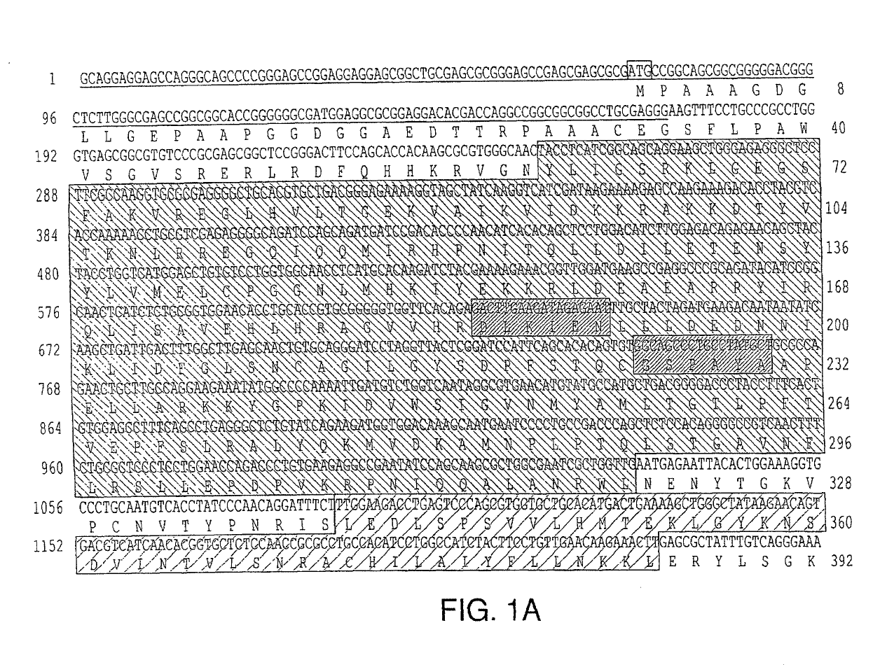

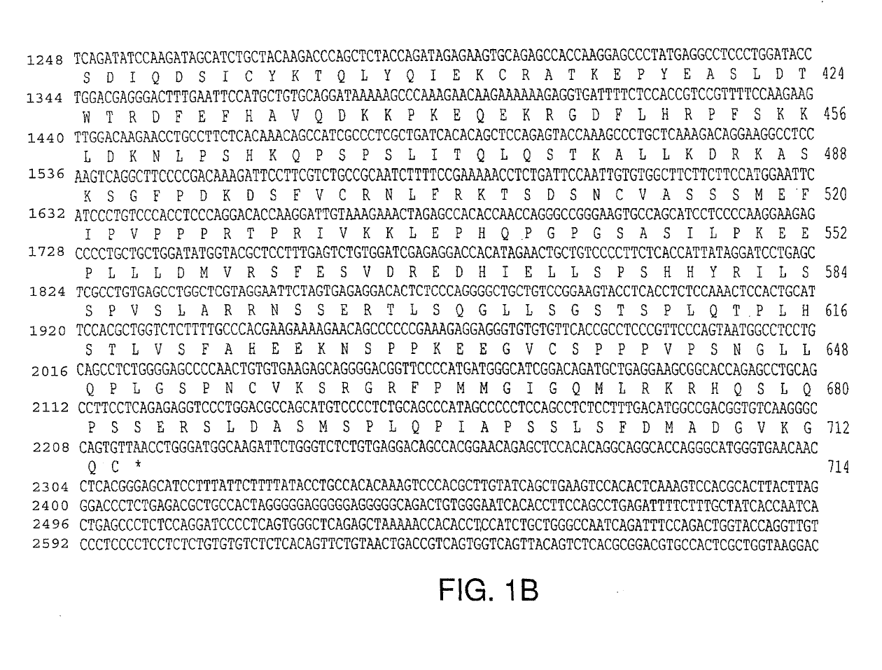

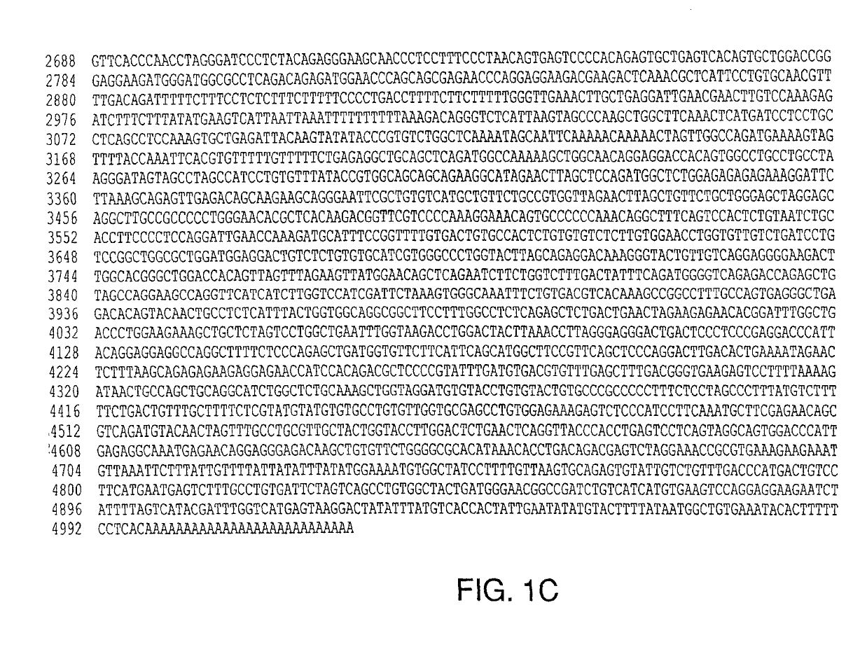

The invention provides the Hunk gene, which has been cloned and fully sequenced as described in the Examples below, and the full length coding sequence of 5026-nucleotides, derived from cDNA is set forth in FIG. 1 and SEQID NO:1. Sequence data have been deposited with the EMBL/GenBank Data Libraries under Accession No. AF 167987.

Hunk possesses an open reading frame (ORF) 2142 nucleotides in length beginning with a putative initiation codon at nucleotide 72. Comparison of the nucleotide sequence surrounding this site with the Kozak consensus sequence (Kozak, Nucleic Acids Res. 15:8125-8132 (1987); Kozak, Cell Biol. 1 15:887-903 (1991)), GCC(A/G)CCAUGG (SEQID NO: 3), reveals matches at positions −4, −3, and −2. The nucleotide sequence of the 5′-UTR and the first 100 nucleotides of the Hunk ORF are extremely GC-rich (˜80%). Other genes bearing such GC-rich sequences have been found to be subject to translational control (Kozak, 1991).

The 3′-UTR of Hunk is 2.8 kb in length, but lacks a canonical AATAAA polyadenylation signal (SEQID NO:4), containing instead the relatively uncommon signal, AATACA (SEQID NO:5), 18 nucleotides upstream from the poly(A)⁺ tract (Bishop et al., Proc. Natl. Acad. Sci. USA, 83:4859-4863 (1986); Herve et al., Brain Res. Mol. Brain Res., 32:125-134 (1995); Myohanen et al., DNA Cell Biol. 10:467-474 (1991); Myohanen et al., DNA Seq., 4:343-346 (1994); Parthasarathy et al., Gene, 191:81-87 (1997); Tokishita et al., Gene, 189:73-78 (1997)).

Several lines of evidence confirmed that the identified Hunk cDNA sequence represents the full-length Hunk ORF. First, Northern hybridization analysis of poly(A)⁺ RNA isolated from mammary epithelial cell lines using a Hunk-specific cDNA probe identified a predominant mRNA species 5.1 kb in length, consistent with the 5025-nucleotide cDNA sequence obtained for clone E8. Secondly, in vitro transcription and translation of clone E8 yielded a polypeptide that is detected by anti-Hunk antisera, that co-migrates with endogenous Hunk, and whose size is consistent with that predicted for the Hunk ORF. Thirdly, comparison of the sequence of clone E8 with a recently isolated human HUNK cDNA clone revealed a high level of homology within the predicted ORF and a lower level of homology 5′ of the predicted initiation codon and 3′ of the predicted termination codon.

Although Hunk mRNA expression levels were found to be markedly up-regulated during early pregnancy, a developmental stage that is characterized by rapid alveolar cell proliferation, multiple lines of evidence suggest that Hunk expression is not simply a correlate of proliferation. For instance, the temporal profile of Hunk expression in the mammary gland during development is distinct from that of bona fide markers of proliferation, such as cyclin A, cyclin Dl, PCNA and PLK (Chodosh et al., 2000). Specifically, the up-regulation of Hunk expression in the mammary gland was confined to early pregnancy, whereas it was found that selected proliferation markers were not only upregulated during early pregnancy, but also during mid-pregnancy, as well as puberty. Moreover, Hunk was not preferentially expressed in proliferative, as compared to nonproliferative, compartments in the mammary gland (i.e. terminal end buds versus ducts during puberty, or alveoli versus ducts during early pregnancy).

Finally, an analysis of actively growing versus confluent or serum-starved mammary epithelial cells revealed no difference in Hunk mKNA levels (Gardner, unpublished). Thus, Hunk expression does not simply reflect the proliferative state of the mammary epithelium, but rather may reflect other developmental pathways or events in the mammary gland.

Hunk up-regulation in the mammary gland during early pregnancy was transient. Thus, the tightly regulated pattern of Hunk expression during pregnancy may be required for normal lobuloalveolar development. This principle was tested by mis-expressing Hunk in the mammary glands of transgenic is mice. Forced overexpression of an MMTV-Hunk: transgene in the mammary epithelium throughout postnatal development resulted in a defect in lobuloalveolar development with molecular abnormalities first discernible during early pregnancy, cellular abnormalities discernible during mid-pregnancy and morphological abnormalities discernible late in pregnancy.

Specifically, Hunk overexpression resulted in a defect in epithelial proliferation that is restricted to mid-pregnancy and a defect in differentiation that was manifest throughout the developmental interval spanning day 6.5 of pregnancy to day 2 of lactation. In contrast, forced overexpression of Hunk in nulliparous animals had no obvious effect on patterns of proliferation or differentiation. Together, this indicated that the defects observed in lobuloalveolar development in MHK3 mice were due to the failure to down-regulate Hunk expression during mid-pregnancy, rather than to Hunk overexpression per se.

The fact that Hunk overexpression inhibited alveolar proliferation during mid-pregnancy was surprising, given the fact Hunk is normally up-regulated in the mammary gland during early pregnancy—the stage of pregnancy associated with maximum alveolar proliferation. Therefore, mechanistically either the normal role of Hunk is the negative regulation of mammary epithelial proliferation during pregnancy, or the inhibitory effect of Hunk on proliferation at day 12.5 of pregnancy is a consequence of overexpression during a developmental stage at which Hunk is normally down-regulated. Alternatively, the developmental profile of endogenous Hunk activity may be different from that of steady-state levels of Hunk mRNA.

While the present work was in progress, a 588-nucleotide portion of the catalytic domain of Hunk was independently isolated by another group and shown to recognize a mRNA approximately 4 kb in length (Korobko et al., Dokl, Akad. Nauk., 354:554-556 (1997)). However, the brief Russian paper offers no additional information to lead one to recognize the function or the utility, or the cloning, characterization, localization, function, or in vivo expression of this molecule. Thus, although a small portion of the full-length gene (<10%) appears to have been sequenced from cDNA, insufficient information is provided by the Russian paper to direct one of ordinary skill to the full-length sequence of Hunk, as provided by the present invention. The Russian gene was neither characterized, nor associated with a relevant utility. Therefore, notwithstanding the disclosure of a partial sequence by the Russians, their disclosure provides insufficient information to be considered an enabling reference with regard to the present invention. Nor would one have used the disclosed gene fragment as a probe to identify the full-length Hunk gene, since there was no reason to consider an association of the fragment with mammary development and carcinogenesis, or with the developmentally regulated and tissue-specific expression related thereto, particularly with regard to pregnancy.

Interestingly, sometime after the discovery of Hunk by the present inventors, Korobko et al., 1997 deposited a 5026-nucleotide sequence in GenBank (Accession No. AF055919) that is only 10 nucleotides shorter at the 5′ end, and in general, 98% identical to Hunk. Even more interesting is the fact that although the present inventors originated the name Hunk, the Russians also referred to their subsequent deposit as Hunk, they did not identify it by the original Russian identifier for the gene. Therefore, the deposit by Korobko et al., 1997 effectively acknowledges the earlier discovery of Hunk by the present inventors or the Russians would not have had prior knowledge of the name Hunk. Consequently, there can be no question that the first inventors of Hunk were the present inventors, not the Russians, who did not produce a full-length clone for Hunk until after the present inventors had already named the gene.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGCG5′ share 50% homology.

In the Examples that follow, two homologous genes were examined, and while not intended to be limited to the exemplified species, standard nomenclature is used. The murine gene is referred to as Hunk, whereas as the human homologue of the same gene is referred to as HUNK. Thus, the invention should be construed to include all Hunk kinase genes that meet the description herein provided, including the human homologue HUNK, as herein described. The nucleotide sequence for human HUNK is set forth as SEQID NO:18, and its corresponding protein expression product as SEQID NO:17. Thus, the invention should be construed to include all Hunk kinase genes that meet the description herein provided, including the human homologue HUNK, as herein described.

The gene encoding Hunk kinase may be isolated as described herein, or by other methods known to those skilled in the art in light of the present disclosure. Alternatively, since, according to the present invention, the gene encoding Hunk has been identified, isolated and characterized, any other Hunk gene which encodes the unique protein kinase described herein may be isolated using recombinant DNA technology, wherein probes derived from Hunk are generated which comprise conserved nucleotide sequences in kinase gene. These probes may be used to identify additional protein kinase genes in genomic DNA libraries obtained from other host strain using the polymerase chain reaction (PCR) or other recombinant DNA methodologies.

An “isolated nucleic acid,” as used herein, refers to a nucleic acid sequence, segment, or fragment which has been separated from the sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins which naturally accompany it in the cell.

Further provided in the present invention is the isolated polypeptide protein kinase product of the Hunk gene and its biological equivalents, which are useful in the methods of this invention. Preferably, the amino acid sequence of the isolated protein kinase is about 70% homologous, more preferably about 80% homologous, even more preferably about 90% homologous and most preferably about 95% homologous to the amino acid sequence Hunk, or its human homologue, HUNK.

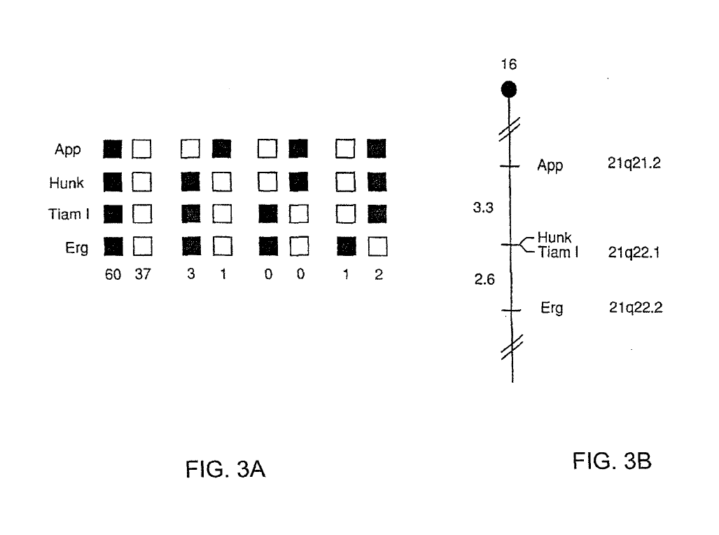

Hunk is located on distal mouse chromosome 16. The distal portion of mouse chromosome 16 shares a region of conserved synteny with human chromosome 21q (summarized in FIG. 3). In particular, Tiam1 has been mapped to 21q22.1. Mutations or segmental trisomy in this region of human chromosome 21 are associated with Alzheimer disease and Down syndrome, respectively. The close linkage between Tiam1 and Hunk in the mouse suggests that the human homologue, HUNK, will map to 21q22, as well. In fact, BLAST alignment of Hunk to sequences in GenBank reveals homology to human genomic DNA sequences cloned from 21q22.1 (gi4835629). This indicates that HUNK also lies within a region of chromosome 21q22, which is believed to contribute to several of the phenotypic features characteristic of Down syndrome (Delabar et al., Eur. J. Hum. Genet., 1:114-124 (1993); Korenberg et al., Proc. Natl. Acad. Sci. USA, 91:4997-5001 (1994); Rahmani et al., Proc. Natl. Acad. Sci. USA, 86:5958-5962 (1989)).

In this regard, it is interesting to note that Hunk is expressed at high levels throughout the brain during murine fetal development, as well as in the adult, with particularly high levels being found in the hippocampus, dentate gyrus, and cortex. However, whether increased Hunk expression in the brain is related to the pathophysiology of Alzheimer disease or Down syndrome is unknown.

Further provided in the present invention is the isolated polypeptide protein kinase product of the Hunk gene and its biological equivalents, which are useful in the methods of this invention. Preferably, the amino acid sequence of the isolated protein kinase is about 70% homologous, more preferably about 80% homologous, even more preferably about 90% homologous and most preferably about 95% homologous to the amino acid sequence Hunk, or its human homologue, HUNK.

Hunk can be purified from natural sources or produced recombinantly using the expression vectors described above in a host-vector system. The proteins also can be produced using the sequence provided in FIG. 1 and methods well known to those of skill in the art. The isolated preparation of Hunk kinase encoded by Hunk may be obtained by cloning and expressing the Hunk gene, and isolating the Hunk protein so expressed, using available technology in the art, and as described herein. The kinase may be purified by following known procedures for protein purification, wherein an immunological, enzymatic or other assay is used to monitor purification at each stage in the procedure.

The conceptual ORF of Hunk comprises 714 amino acids and encodes a polypeptide of predicted molecular mass 79.6 kDa, see FIG. 1 and SEQID NO:2. Frequently rounded herein to a size of ˜80 kDa, this polypeptide is divisible into an amino-terminal domain of 60 amino acids, a 260-amino-acid kinase catalytic domain, and a 394-amino-acid carboxyl-terminal domain. The carboxyl-terminal domain of Hunk contains a 46-amino-acid conserved motif located 18 amino acids C-terminal to the catalytic domain that is homologous to the previously described SNF1 homology region, or SNH (Becker et al., 1996). The 330 amino acids that are carboxyl to the SNH, lack homology to other known proteins.

Consistent with this, antisera that specifically immunoprecipitate Hunk co-immunoprecipitate phosphotransferase activity, and overexpression of Hunk in mammary epithelial cells increased the level of this phosphotransferase activity. Hunk expression in the mouse is developmentally regulated and tissue-specific both during fetal development and in the adult. Interestingly, within multiple tissues Hunk expression is restricted to sub-sets of cells within specific cellular compartments, predicting a role for Hunk in developmental processes in multiple tissues.

The putative catalytic domain of Hunk contains each of the invariant amino acid motifs characteristic of all protein kinases, as well as sequences specific to serine/threonine kinases (Hanks et al., Methods Enzymol. 200:38-79 (1991); Hanks et al., Science 241:42-52 (1988)). In particular, the DLKPEN to motif (SEQID NO:6) in subdomain VIB of the Hunk cDNA predicted serine/threonine kinase specificity (ten Dijke et al., Progr. Growth Factor Res. 5:55-72 (1994)). Hunk also contains the serine/threonine consensus sequence in subdomain VIII N-terminal to the APE motif, which is conserved among all protein kinases. In addition, several amino acids in subdomains I, VII, VIII, IX, X, and XI that are conserved among tyrosine kinases are absent from the Hunk ORF. Thus, the primary sequence analysis further confirms that Hunk encodes a functional serine/threonine kinase, not a tyrosine kinase.

Moreover, the observation that anti-Hunk antisera appear to recognize a single polypeptide species in lysates from cells known to express both transcripts provides evidence that the present invention comprises the isolation of the entire ORF and contain the complete coding region. Taken together, these findings suggest that the cDNA clones isolated represent a full-length Hunk transcript, and that the 5.6-kb Hunk mRNA contains additional 5′ or 3′ untranslated sequence. The difficulties associated with identifying cDNA clones containing additional 5′ sequence may be related to the GC-rich nature of the 5′ UTR of Hunk, and the tendency of reverse transcriptase to terminate prematurely in such regions. Alternately, the difference in size between the 5.1- and the 5.6-kb transcripts may be due to utilization of an alternate downstream polyadenylation site during mRNA processing.

A “biological equivalent” is intended to mean any fragment of the nucleic acid or protein, or a mimetic (protein and non-protein mimetic) also having the ability to alter Hunk kinase activity using the assay systems described and exemplified herein. For example, purified Hunk polypeptide can be contacted with a suitable cell, as described above, and under such conditions that its kinase activity is inhibited, or in some cases, it may be enhanced. By “inhibited,” is meant a change in kinase activity that is measurably less than the activity exhibited before contact with the subject cell; by “enhances,” is meant a change in kinase activity that is measurably greater than the activity exhibited before contact with the subject cell.

The protein is used in substantially pure form. As used herein, the term “substantially pure,” or “isolated preparation of a polypeptide” is meant that the protein is substantially free of other biochemical moieties with which it is normally associated in nature. Typically, a compound is isolated when at least 25%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis or HPLC analysis.

The present invention also provides for analogs of proteins or peptides encoded by Hunk or its human homologue, HUNK. Analogs can differ from naturally occurring proteins or peptides by conservative amino acid sequence differences or by modifications which do not affect sequence, or by both. It is understood that limited modifications can be made to the primary sequence of the Hunk sequence as shown in FIG. 1 and used in this invention without destroying its biological function, and that only the active portion of the entire primary structure may be required in order to effect biological activity. It is further understood that minor modifications of the primary amino acid sequence may result in proteins, which have substantially equivalent or enhanced function as compared to the molecule within the vector. These modifications may be deliberate, e.g., through site-directed mutagenesis, or may be accidental, e.g., through mutation in hosts. All of these modifications are included in the present invention, as long as the Hunk kinase activity is retained essentially as in its native form.

For example, conservative amino acid changes may be made, which although they alter the primary sequence of the protein or peptide, do not normally alter its function. Conservative amino acid substitutions typically include substitutions within the following groups: glycine and alanine; valine, isoleucine, and leucine; aspartic acid and glutamic acid; asparagine and glutamine; serine and threonine; lysine and arginine; or phenylalanine and tyrosine.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps, e.g., by exposing the polypeptide to enzymes which affect glycosylation, e.g., mammalian glycosylating or deglycosylating enzymes. Also embraced are sequences that have phosphorylated amino acid residues, e.g., phosphotyrosine, phosphoserine, or phosphothreonine. Also included are polypeptides that have been modified using ordinary molecular biological techniques so as to improve their resistance to proteolytic degradation or to optimize solubility properties. Analogs of such polypeptides include those containing residues other than naturally-occurring L-amino acids, e.g., D-amino acids or non-naturally occurring synthetic amino acids. The peptides of the invention are not limited to products of any of the specific exemplary processes listed herein.

In addition to substantially full-length polypeptides, the present invention provides for enzymatically active fragments of the polypeptides. A Hunk-specific polypeptide is “enzymatically active” if it is characterized in substantially the same manner as the naturally encoded protein in the assays described below.

As used herein, the term “fragment,” as applied to a polypeptide, will ordinarily be at least about 20 contiguous amino acids, typically at least about 50 contiguous amino acids, more typically at least about 70 continuous amino acids, usually at least about 100 contiguous amino acids, more preferably at least about 150 continuous amino acids in length.

Hunk is spatially and temporally regulated during murine mammary development Hunk is expressed at high levels in the embryo during mid-gestation as cells are rapidly proliferating and differentiating and is down-regulated in the embryo prior to parturition. During fetal development, Hunk mRNA is expressed in a tissue-specific manner and is restricted to particular compartments within expressing tissues. Similarly, Hunk is also expressed in a tissue-specific manner in the adult mouse, and its expression is restricted to subsets of cells within these tissues, with highest levels observed in ovary, lung and brain.

Functionally, the temporal and spatial regulation of Hunk has been characterized in various murine and human tissues, as summarized in Table 1.

TABLE 1
Hunk Expression

| | |
|---|---|
| Expression | Expressed in rapidly proliferating cells in vivo; Appears to |
| | negatively regulate proliferation in vivo. |
| Breast Cancer in | overexpressed in cell lines from tumors induced by the |
| Transgenic Mice | Neu/ErbB2/Her2 and Ras oncogenes: Not expressed in cell lines |
| | from tumors induced by the c-Myc or Int-2 oncogenes. |
| Expression in | Expression is highly heterogeneous in cell lines from a wide |
| Human Cancer | variety of tumor types; expressed at high levels (or at undetectable |
| Cell Lines | levels) in a subset of breast, colon, ovarian, prostate, lung, CNS, |
| | cervical and renal cancer cell lines, |
| Human Breast | Underexpressed in 50% of primary breast cancers compared to |
| Cancer | normal tissue; overexpressed in approximately 25% of human |
| | breast cancers compared to normal tissue. |
| Human Colon | Overexpressed in moderately differentiated colon cancers |
| Cancer | compared to well differentiated colon cancers and compared to |
| | benign tissue. |
| Human Ovarian | Overexpressed in poorly differentiated and moderately |
| Cancer | differentiated ovarian cancers compared to well differentiated |
| | ovarian cancers and benign tissue. |
| Other Human | overexpressed in a subset of endometrial and lung cancers |
| Cancers | compared to benign tissue. Highly expressed in a subset of |
| | carcinoid tumors. |

When compared with other previously isolated protein kinases, multiple sequence alignment showed that the kinase catalytic domain of Hunk displays highest homology to the S. cerevisiae SNF1 family of serine/threonine kinases. However, Hunk does not appear to belong to the most recognized SNF1 subfamily of protein kinases, rather Hunk appears to represent a new branch of the SNF1 family tree.

In addition to the conserved kinase catalytic domain, SNF1-related protein kinases contain the SNH region of homology, or SNF1 homology domain (Becker et al., 1996). Although amino acids in this motif are conserved in all SNF1 family members, the functional significance of the SNH domain is unknown. Multiple sequence alignment analysis revealed that the SNH is anchored approximately 20 amino acids carboxyl-terminal to the kinase domain, spans approximately 45 amino acids, and extends further toward the amino terminus than previously reported. The present consensus identified amino acids exhibiting greater than 70% conservation among the SNF1 family members shown, as well as residues that are specific for particular SNF1 kinase subfamilies.

Although most conserved residues are shared among all SNF1 family members, some residues are relatively specific for a particular subfamily. For example, the consensus amino acid at position 32 of the SNH is glutamine in subfamily I SNF1 kinases, and tyrosine in subfamily II kinases. Subclass-specific residues are also found at positions 37 (alanine versus valine) and 45 (lysine/arginine versus asparagine).

On the other hand, other than its kinase and SNH domains, Hunk displayed no detectable homology to other members of the SNF1 family or to other known molecules.

Since the distance between the catalytic domain and the SNH is conserved and since many kinases contain autoregulatory domains, it is plausible that the SNH domain functions to regulate kinase activity (Yokokura et al., 1995). Consistent with this speculation is the presence of weak homology between the SNH domain of SNF1 kinases and the autoinhibitory domain of the closely related family of calcium-calmodulin regulated kinases (data not shown). This homology does not extend into the adjacent calmodulin-binding region, consistent with the observation that SNF1 kinases are not regulated by calcium. Regardless, the presence of the SNH domain in all SNF1 kinases raises the possibility that members of this family of molecules may be regulated by a common mechanism.

After isolating the Hunk kinase (Example 1) and cloning and characterizing Hunk as a novel member of the family of SNF1-related protein kinases (Example 2), four founder mice were identified in Example 3 as harboring the MMTV-Hunk transgene in DNA that passed the transgene to offspring in a Mendelian fashion. When screened for transgene expression by Northern hybridization and RNase protection analysis. One founder line, MHK3, was identified that expressed the MMTV-Hunk transgene at high levels, and it became the focus of comparisons with endogenous Hunk expression during all stages of postnatal mammary development.

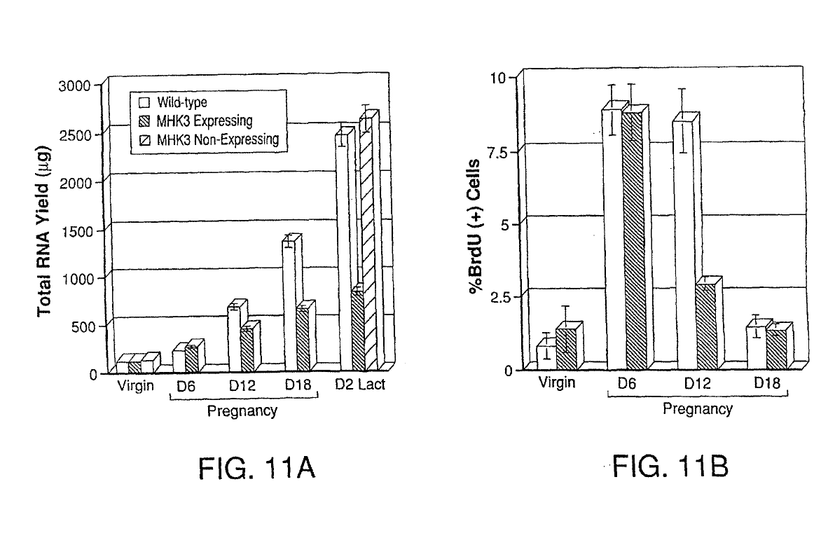

Defects have been demonstrated in both mammary epithelial proliferation and differentiation in MHK3 animals during pregnancy. For example, the lower total RNA yield obtained from transgenic glands as compared with wild type glands during late pregnancy and lactation probably reflects, in part, the reduced epithelial cell content of MHK3 transgenic glands, since the increase in total RNA present in the mammary gland during lobuloalveolar development is a result both of increases in epithelial cell number and increases in expression of milk protein genes on a per-cell basis (FIG. 11B). Consequently, there was the consideration that the decreased expression of markers for mammary epithelial differentiation observed in MHK3 animals during pregnancy and lactation was a consequence of the decreased alveolar proliferation evident in MHK3 mice at day 12.5 of pregnancy, and the resulting decrease in epithelial cell mass. However, several lines of evidence indicate that the abnormalities in mammary epithelial differentiation observed in MHK3 animals cannot be explained by a decrease in epithelial cell mass. First, the fact that defects in alveolar differentiation in MHK3 animals actually precede the reduction in epithelial proliferation that occurs at day 12.5 strongly argues that defects in differentiation cannot solely be a consequence of defects in proliferation. In addition, RNA extracted from a mammary gland composed of a smaller number of appropriately differentiated epithelial cells would be predicted to give rise to a normal distribution of milk protein gene expression (i.e., early versus late), and to normal levels of expression of milk protein genes when normalized to epithelial cell content.

In contrast, the present invention demonstrates that that both the level and the composition of milk protein RNA produced by the mammary glands of MHK3 animals during pregnancy and lactation is abnormal, even after controlling for differences in epithelial content between wild type and transgenic glands. Consistent with this conclusion, the morphology of the alveolar epithelial cells present in the mammary glands of MHK3 animals at day 18.5 of pregnancy is less differentiated compared with those present in their wild type counterparts. Thus, the reduced expression of differentiation markers in MHK3 transgenic glands reflects the less differentiated state of the mammary epithelial cells present, rather than a reduced number of appropriately differentiated mammary epithelial cells. As such, the data show that the defects in differentiation that occur in MHK3 animals as a consequence of Hunk overexpression are separable from and independent of the defects in proliferation that occur in these animals.

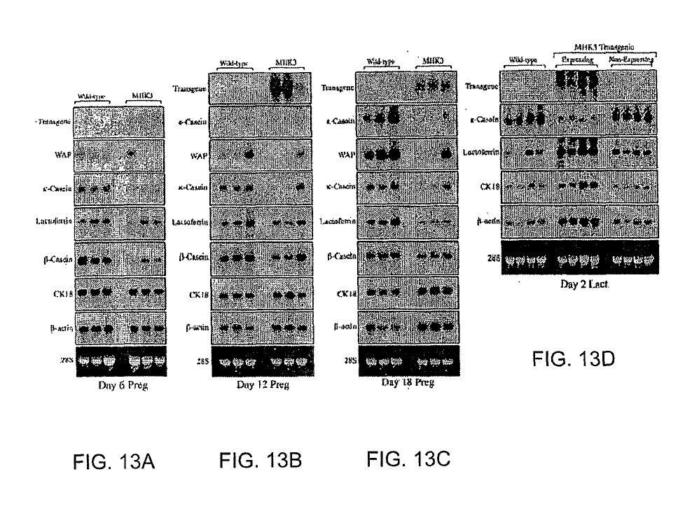

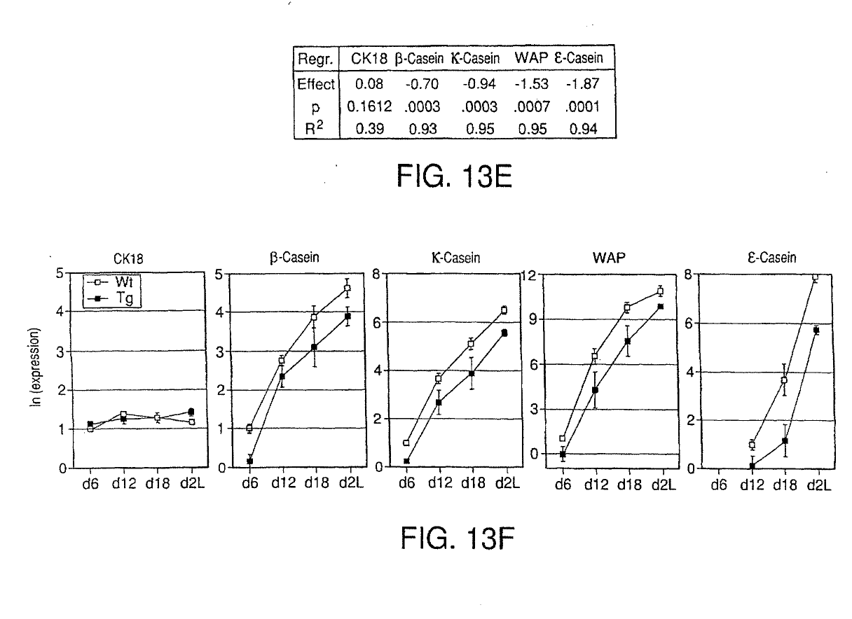

It is important to note that during pregnancy and lactation, a similar magnitude of reduction in the expression of differentiation markers was observed in the mammary glands of MHK3 animals compared with wild type animals, regardless of whether levels of expression of milk protein genes were normalized to β-actin or to the epithelial cell marker, cytokeratin 18 (FIG. 13, and data not shown). In other words, when normalized to β-actin expression, cytokeratin 18 expression levels do not differ between MHK3 transgenic animals and wild type animals at any stage of lobuloalveolar development, reflecting the fact that mammary epithelial cells contribute the vast majority of RNA to the total RNA pool during pregnancy and lactation. This observation explains why cytokeratin 18 levels show little change during pregnancy when normalized to β-actin expression. Thus, normalizing mRNA expression levels to β-actin mRNA levels itself effectively controls for the decreases in epithelial cell content that occur in MHK3 animals. In addition, these kinases are useful as diagnostic tools, as markers to assess a patient's illness, and/or prognostically, to determine how aggressively, or with what agent a diagnosed case of cancer should be treated.

The invention further provides a method of identifying a therapeutic compound having activity to affect Hunk by screening a test compound for its ability to modulate the expression or activity of Hunk. In one embodiment of the invention, a method includes analysis of the effect of a compound on Hunk activity by comparing the result of: 1) contacting a cell comprising Hunk with a test compound with the result obtained by 2) contacting a cell lacking Hunk with the test compound. In an embodiment, a method includes providing a first cell comprising Hunk and measuring the metastatic activity of the cell under defined culture conditions to obtain a metastatic value. Subsequently, the cell is contacted with the test compound and a second metastatic value is obtained. The difference between the first and second measured metastatic values provides an “inhibitory value,” which is a relative measure of the degree of inhibition of Hunk when compared to the inhibitory value obtained by performing the method of the invention using a cell devoid of Hunk.

That is, the difference in metastatic activity between a Hunk-positive and a Hunk-negative cell, wherein the comparison is made both before and after treatment of the cells with a test compound, will provide a relative measure of the effect of the test compound on Hunk. By way of a non-limiting example, a greater inhibitory value obtained by treating a Hunk-positive cell with a test compound than that obtained by treating a Hunk-negative cell with the test compound demonstrates a Hunk-inhibitory effect of the test compound. Methods of assaying for metastatic potential of a cell are known in the art, and are also described, in part, elsewhere herein.

Methods of the invention can be practiced in vitro, ex vivo or in vivo. When the method is practiced in vitro, the expression vector, protein or polypeptide can be added to the cells in culture or added to a pharmaceutically acceptable carrier as defined below. In addition, the expression vector or Hunk DNA can be inserted into the target cell using well known techniques, such as transfection, electroporation or microinjection. By “target cell” is meant any cell that is the focus of examination, delivery, therapy, modulation or the like by, or as a result of, activation, inactivation, expression or changed expression of Hunk or the nucleotide sequence encoding same, or any cell that effects such modulation, activation, inactivation or the like in the kinase or gene encoding it.

Compounds which are identified using the methods of the invention are candidate therapeutic compounds for treatment of disease states or carcinomas in patients caused by or associated with Hunk or by a cell type related to the activation of Hunk, such as an epithelial cell type as yet unidentified which activates or is activated by the a cancerous condition in the subject, particularly in a human patient. By “patient” is meant any human or animal subject in need or treatment and/or to whom the compositions or methods of the present invention are applied. It is preferred that in a preferred embodiment of the invention, the patient is a mammal, more preferred that it is a veterinary animal, most preferred that it is a human.

The use of the compositions and methods in vitro provides a powerful bioassay for screening for is drugs which are agonists or antagonists of Hunk function in these cells. Thus, one can screen for drugs having similar or enhanced ability to prevent or inhibit Hunk kinase activity. It also is useful to assay for drugs having the ability to inhibit carcinogenesis, particularly in the breast. The in vitro method further provides an assay to determine if the method of this invention is useful to treat a subject's pathological condition or disease that has been linked to enhanced Hunk expression, to the developmental stages associated with up-regulation of Hunk, or to a cancerous condition, particularly in the breast or other tissues in which Hunk is highly expressed.

Generally the term “activity,” as used herein, is intended to relate to Hunk kinase activity, as well as to the ability of Hunk to enhance or increase metastasis of a cell comprising Hunk, and an “effective amount” of a compound with regard to Hunk kinase activity means a compound that modulates (inhibits or enhances) that Hunk activity. However, the term “activity” as used herein with regard to a compound, also means the capability of that compound, that in some way affects Hunk kinase activity, to also destroy or inhibit the uncontrolled growth of cells, particularly cancerous cells, particularly in a tumor, or which is capable of inhibiting the pathogenesis, i.e., the disease-causing capacity, of such cells. Similarly, an “effective amount” of such a compound is that amount of the compound that is sufficient to destroy or inhibit the uncontrolled growth of cells, particularly cancerous cells, particularly in a tumor, or which is capable of inhibiting the pathogenesis, i.e., the disease-causing capacity, of such cells. In the alternative, in the case of an enhancing effect, and “effective amount” is that amount of the compound that is sufficient to enhance or increase a desired effect as compared with a corresponding normal cell, or a benign cell.

Acceptable “pharmaceutical carriers” are well known to those of skill in the art and can include, but are not limited to any of the standard pharmaceutical carriers, such as phosphate buffered saline, water and emulsions, such as oil/water emulsions and various types of wetting agents.

The assay method can also be practiced ex vivo. Generally, a sample of cells, such as those in the mammary gland, blood or other relevant tissue, can be removed from a subject or animal using methods well known to those of skill in the art. An effective amount of antisense Hunk nucleic acid or a Hunk inhibitor or suspected Hunk inhibitor is added to the cells and the cells are cultured under conditions that to favor internalization of the nucleic acid by the cells. The transformed cells are then returned or reintroduced to the same subject or animal (autologous) or one of the same species (allogeneic) in an effective amount and in combination with appropriate pharmaceutical compositions and carriers.

As used herein, the term “administering” for in vivo and ex vivo purposes means providing the subject with an effective amount of the nucleic acid molecule or polypeptide effective to prevent or inhibit Hunk kinase activity in the target cell.

In each of the assays described, control experiments may include the use of mutant strains or cells types that do not encode Hunk. Such strains are generated by disruption of the Hunk gene, generally in vitro, followed by recombination of the disrupted gene into the genome of host cell using technology which is available in the art of recombinant DNA technology as applied to the generation of such mutants in light of the present disclosure. The host may include transgenic hosts.

In another aspect of the invention, RNAi is useful for inhibiting Hunk activity. Briefly, RNAi involves the administration of homologous double stranded RNA (dsRNA) to a cell, wherein the dsRNA specifically targets the transcription product of a target gene, resulting in the inhibition of expression of the target gene. Methods of preparing materials and conducting RNAi experiments and assays are known in the art, and will therefore not be discussed in detail herein (see, eg., U.S. Pat. No. 6,506,559 of Fire et al.).

Accordingly, by way of a non-limiting example, dsRNA that is specific for the gene product of Hunk is useful for the administration to a cell, for the purpose of inhibiting the expression of Hunk in the cell. Inhibition of Hunk using such an inhibitor, referred to herein as an “interfering RNA,” will result in a decrease in Hunk activity in the cell. As described in detail elsewhere herein, the Hunk activity is required for metastasis of a cancer cell. Inhibition of Hunk using an RNAi technique is therefore useful for inhibiting metastasis of a tumor cell, among other things. Additionally, an RNAi technique can be used to inhibit mammary tumor formation that is induced by the Neu oncogene. This is because, as described in detail elsewhere herein, Hunk is required for mammary tumor formation induced by the Neu oncogene.

In one aspect of the assay method of the invention, a compound is assessed for therapeutic activity by examining the effect of the compound on Hunk kinase activity. In this instance, the test compound is added to an assay mixture designed to measure protein kinase activity. The assay mixture may comprise a mixture of cells that express Hunk, a buffer solution suitable for optimal activity of the kinase, and the test compound. Controls may include the assay mixture without the test compound and the assay mixture having the test compound. The mixture is incubated for a selected length of time and temperature under conditions suitable for expression of the Hunk kinase as described herein, whereupon the reaction is stopped and the presence or absence of the kinase, or its overexpression is assessed, also as described herein.

Compounds that modulate the Hunk kinase activity, either by enhancing or inhibiting the activity, are easily identified in the assay by assessing the production of the expression product by the methods exemplified in the presence or absence of the test compound. A lower level, or minimal amounts of Hunk in the presence of the test compound compared with the absence of the test compound in the assay mixture is an indication that the test compound inhibits the selected kinase activity. Similarly, an increased, or significantly increased level, or higher amounts of Hunk in the presence of the test compound compared with the absence of the test compound in the assay mixture is an indication that the test compound enhances or increases the selected kinase activity.

The method of the invention is not limited by the type of test compound used in the assay. The test compound may thus be a synthetic or naturally-occurring molecule, which may comprise a peptide or peptide-like molecule, or it may be any other molecule, either small or large, which is suitable for testing in the assay. In another embodiment, the test compound is an antibody or antisense molecule directed against Hunk kinase, or its human homologue, or other homologues thereof, or even directed against active fragments of Hunk kinase molecules.

In one aspect, a compound useful for inhibiting the kinase activity of any Hunk protein is a protein kinase inhibitor. In another aspect, a compound is a serine/threonine protein kinase inhibitor. Examples of such inhibitors useful in the present invention include, but are not limited to, a cyclic AMP derivative, a protein kinase A inhibitor, a protein kinase C inhibitor, a protein kinase G inhibitor, a calmodulin kinase inhibitor, staurosporine, an MLCK inhibitor, and the like.

As will be understood by the skilled artisan, when armed with the disclosure set forth herein, any serine/threonine kinase inhibitor can be modified, using chemical methods known in the art, in order to enhance or diminish the specificity of binding of such inhibitor with any Hunk protein. That is, using methods of chemical design and modification, the skilled artisan would understand, based on the present disclosure, how to modulate the binding properties of a kinase inhibitor with respect to a Hunk protein. By modulating the Hunk-binding properties of an inhibitor, the skilled artisan can create inhibitors that bind Hunk more tightly or more weakly. By doing so, Hunk inhibitors can be created that provide more potent inhibition or that provide weaker inhibition of Hunk activity. Based on the disclosure set forth herein, it will be understood that a Hunk inhibitor may inhibit the kinase activity of Hunk, or may to otherwise inhibit the metastatic potential of Hunk. By way of a non-limiting example, an inhibitor may inhibit the metastatic potential of Hunk through inhibition of the kinase activity, through inhibition of Hunk in a mode other than through inhibition of the kinase activity, or through a combination of two or more distinct modes of inhibition of Hunk, one of which may or may not be inhibition of the kinase activity.

As will be further understood based on the present disclosure, any compound that binds to a Hunk protein, either now known or discovered in the future, may be useful to inhibit the activity of a Hunk protein. Using the methods set forth herein, the skilled artisan will understand how to assay a compound for Hunk inhibitory activity, thereby identifying a Hunk inhibitor.

Compounds which inhibit Hunk kinase activity in vitro are then tested for activity directed against HUNK kinase in vivo in humans. Essentially, the compound is administered to the human by any one of the routes described herein, and the effect of the compound is assessed by clinical and symptomatic evaluation. Such assessment is well known to the practitioner in the field of developmental biology or those studying the effect of cancer drugs. Compounds may also be assessed in an in vivo animal model, as herein described.

Precise formulations and dosages will depend on the nature of the test compound and may be determined using standard techniques, by a pharmacologist of ordinary skill in the art.

The compound may also be assessed in non-transgenic animals to determine whether it acts through inhibition of Hunk kinase activity in vivo, or whether it acts via another mechanism. To test this effect of the test compound on activity, the procedures described above are followed using non-transgenic animals instead of transgenic animals.

This invention also provides vector and protein compositions useful for the preparation of medicaments which can be used for preventing or inhibiting Hunk kinase activity, maintaining cellular function and viability in a suitable cell, or for the treatment of a disease characterized by the unwanted death of target cells or uncontrolled cell amplification, particularly as in a cancer.

The nucleic acid can be duplicated using a host-vector system and traditional cloning techniques with appropriate replication vectors. A “host-vector system” refers to host cells which have been transfected with appropriate vectors using recombinant DNA techniques. The vectors and methods disclosed herein are suitable for use in host cells over a wide range of eukaryotic organisms. This invention also encompasses the cells transformed with the novel replication and expression vectors described herein.

The Hunk gene, made and isolated using the above methods, can be directly inserted into an expression vector, e.g., as in the Examples that follow, and inserted into a suitable animal or mammalian cell, such as a mouse or mouse cell or that of a guinea pig, rabbit, simian cell, rat, or acceptable animal host cells, or into a human cell.

A variety of different gene transfer approaches are available to deliver the Hunk gene into a target cell, cells or tissues. Among these are several non-viral vectors, including DNA/liposome complexes, and targeted viral protein DNA complexes. In addition, the Hunk nucleic acid also can be incorporated into a “heterologous DNA” or “expression vector” for the practice of this invention. The term “heterologous DNA” is intended to encompass a DNA polymer, such as viral vector DNA, plasmid vector DNA, or cosmid vector DNA. Prior to insertion into the vector, it is in the form of a separate fragment, or as a component of a larger DNA construct, which has been derived from DNA isolated at least once in substantially pure form as described above, i.e., free of contaminating endogenous materials and in a quantity or concentration enabling identification, manipulation, and recovery of the segment and its component nucleotide sequences by standard biochemical methods, for example, using a cloning vector.

As used herein, “recombinant” is intended to mean that a particular DNA sequence is the product of various combination of cloning, restriction, and ligation steps resulting in a construct having a sequence distinguishable from homologous sequences found in natural systems. Recombinant sequences can be assembled from cloned fragments and short oligonucleotides linkers, or from a series of oligonucleotides.

As noted above, one means to introduce the nucleic acid into the cell of interest is by the use of a recombinant expression vector. “Recombinant expression vector” includes vectors which are capable of expressing DNA sequences contained therein, where such sequences are operatively linked to other sequences capable of effecting their expression. It is implied, although not always explicitly stated, that these expression vectors must be replicable in the host organisms either as episomes or as an integral part of the chromosomal DNA. In sum, “expression vector” is given a functional definition, and any DNA sequence which is capable of effecting expression of a specified DNA sequence disposed therein is included in this term as it is applied to the specified sequence.

Suitable expression vectors include viral vectors, including adenoviruses, adeno-associated viruses, retroviruses, cosmids and others. Adenoviral vectors are a particularly effective means for introducing genes into tissues in vivo because of their high level of expression and efficient transformation of cells both in vitro and in vivo. Thus, in a preferred embodiment of the invention, a disease state or cancer in a patient caused by or related to the expression of Hunk, may be effectively treated by gene transfer by administering to that patient an effective amount of Hunk or an acceptable species-specific homologue thereof, wherein the gene is delivered to the patient by an adenovirus vector using recognized delivery methods.

The invention also relates to eukaryotic host cells comprising a vector comprising Hunk or a homologue thereof, particularly the human homologue, according to the invention. Such a cell is advantageously a mammalian cell, and preferably a human cell, and can comprise said vector in integrated form in the genome, or preferably in non-integrated (episome) form. The subject of the invention is also the therapeutic or prophylactic use of such vector comprising Hunk or a homologue thereof, particularly the human homologue, or eukaryotic host cell.

In addition, the present invention relates to a pharmaceutical composition comprising as therapeutic or prophylactic agent a vector comprising Hunk or a homologue thereof, particularly the human homologue according to the invention, in combination with a vehicle, which is acceptable for pharmaceutical purposes. Alternately it comprises an antisense Hunk molecule, or a Hunk inhibitor molecule or suspected Hunk inhibitor molecule.

The composition according to the invention is intended especially for the preventive or curative treatment of disorders, such as hyperproliferative disorders and cancers, including those induced by carcinogens, viruses and/or dysregulation of oncogene expression; or by the activation of Hunk, or its homologue; or by expression or amplification of a presently unknown cell type, such as an epithelial cell, which is activated or transformed in the breast as a result of or related to Hunk expression, or for which Hunk expression is an indicator. The treatment of cancer (before or after the appearance of significant symptoms) is particularly preferred.

The phrase “therapeutically effective amount” is used herein to mean an amount sufficient to reduce by at least about 15%, preferably by at least 50%, more preferably by at least 90%, and most preferably complete remission of a hyperproliferative disease or cancer of the host. Alternatively, a “therapeutically effective amount” is sufficient to cause an improvement in a clinically significant condition in the host. In the context of the present invention, a therapeutically effective amount of the expression product of Hunk or a homologue thereof, particularly the human homologue, is that amount which is effective to treat a proliferative disease or tumor or other cancerous condition, in a patient or host, thereby effecting a reduction in size or virulence or the elimination of such disease or cancer. Preferably, administration or expression of an “effective” amount of the expression product of Hunk or a homologue thereof, particularly the human homologue resolves the underlying infection or cancer. A therapeutically effective amount” also relates to non-Hunk molecules, such as, but not limited to, protein kinase inhibitors.

A pharmaceutical composition according to the invention may be manufactured in a conventional manner. In particular, a therapeutically effective amount of a therapeutic or prophylactic agent is combined with a vehicle such as a diluent. A composition according to the invention may be administered to a patient (human or animal) by aerosol or via any conventional route in use in the field of the art, especially via the oral, subcutaneous, intramuscular, intravenous, intraperitoneal, intrapulmonary, intratumoral, intratracheal route or a combination of routes. The administration may take place in a single dose or a dose repeated one or more times after a certain time interval.

The appropriate administration route and dosage vary in accordance with various parameters, for example with the individual being treated or the disorder to be treated, or alternatively with the gene(s) of interest to be transferred. The particular formulation employed will be selected according to conventional knowledge depending on the properties of the tumor, or hyperproliferative target tissue and the desired site of action to ensure optimal activity of the active ingredients, i.e., the extent to which the protein kinase reaches its target tissue or a biological fluid from which the drug has access to its site of action. In addition, these viruses may be delivered using any vehicles useful for administration of the protein kinase, which would be known to those skilled in the art. It can be packaged into capsules, tablets, etc. using formulations known to those skilled in the art of pharmaceutical formulation.

Dosages for a given host can be determined using conventional considerations, e.g., by customary comparison of the differential activities of the subject preparations and a known appropriate, conventional pharmacological protocol. Generally, a pharmaceutical composition according to the invention comprises a dose of the protein kinase according to the invention of between 10⁴ and 10¹⁴, advantageously 10⁵ and 10¹³, and preferably 10⁶ and 10¹¹.

A pharmaceutical composition, especially one used for prophylactic purposes, can comprise, in addition, a pharmaceutically acceptable adjuvant, carrier, fillers or the like. Suitable pharmaceutically acceptable carriers are well known in the art. Examples of typical carriers include saline, buffered saline and other salts, liposomes, and surfactants. The adenovirus may also be lyophilized and administered in the forms of a powder. Taking appropriate precautions not to denature the protein, the preparations can be sterilized and if desired mixed with auxiliary agents, e.g., lubricants, preservatives, stabilizers, wetting agents, emulsifiers, salts for influencing osmotic pressure, buffers, and the like that do not deleteriously react with the active virus. They also can be combined where desired with other biologically active agents, e.g., antisense DNA or mRNA.

The compositions and methods described herein can be useful for preventing or treating cancers of a number of types, including but not limited to breast cancer, sarcomas and other neoplasms, bladder cancer, colon cancer, lung cancer, pancreatic cancer, gastric cancer, cervical cancer, ovarian cancer, brain cancers, various leukemias and lymphomas. One would expect that any other human tumor cell, regardless of expression of functional p53, would be subject to treatment or prevention by the methods of the present invention, although the particular emphasis is on mammary cells and mammary tumors. The invention also encompasses a method of treatment, according to which a therapeutically effective amount of the protein kinase, or a vector comprising same according to the invention is administered to a patient requiring such treatment.

Also useful in conjunction with the methods provided in the present invention would be chemotherapy, phototherapy, anti-angiogenic or irradiation therapies, separately or combined, which maybe used before or during the enhanced treatments of the present invention, but will be most effectively used after the cells have been sensitized by the present methods. As used herein, the phrase “chemotherapeutic agent” means any chemical agent or drug used in chemotherapy treatment, which selectively affects tumor cells, including but not limited to, such agents as adriamycin, actinomycin D, camptothecin, colchicine, taxol, cisplatinum, vincristine, vinblastine, and methotrexate. Other such agents are well known in the art.

As described above, the agents encompassed by this invention are not limited to working by any one mechanism, and may for example be effective by direct poisoning, apoptosis or other mechanisms of cell death or killing, tumor inactivation, or other mechanisms known or unknown. The means for contacting tumor cells with these agents and for administering a chemotherapeutic agent to a subject are well known and readily available to those of skill in the art.

As also used herein, the term “irradiation” or “irradiating” is intended in its broadest sense to include any treatment of a tumor cell or subject by photons, electrons, neutrons or other ionizing radiations. These radiations include, but are not limited to, X-rays, gamma-radiation, or heavy ion particles, such as alpha or beta particles. Moreover, the irradiation may be radioactive, as is commonly used in cancer treatment and can include interstitial irradiation. The means for irradiating tumor cells and a subject are well known and readily available to those of skill in the art.

The protein kinase of the present invention can also be used to express immuno-stimulatory to proteins that can increase the potential anti-tumor immune response, suicide genes, anti-angiogenic proteins, and/or other proteins that augment the efficacy of these treatments.

Further still, the present invention identifies gene expression signatures in both mouse and human breast cancers that are associated with expression of the SNF1-related kinase, HUNK. As exemplified herein, these signatures strongly predict metastasis-free survival in women with breast cancer. This is is because the increased risk of tumor recurrence associated with the HUNK-expression signature described for the first time herein is largely independent of prognostic indicators currently used, as well as previously defined metastatic signatures. This increased risk is greater than that associated with HER2/neu amplification, tumor grade, or ER status. In another aspect of the invention, through germline deletion in mice, it is demonstrated for the first time herein that Hunk is required for efficient mammary tumor metastasis, that Hunk kinase activity is required for this effect, that Hunk is required for efficient mammary tumor formation, and that Hunk is not required for normal mammalian development or physiology. Therefore, Hunk kinase represents a safe target for therapeutic intervention. Taken together, the data disclosed herein demonstrates for the first time that HUNK plays an essential role in breast cancer formation and metastasis.

Therefore, in an embodiment of the invention, inhibition of Hunk activity is useful to prevent Neu oncogene-induced mammary tumor formation. This is because it has also been shown herein for the first time that Hunk is required for mammary tumor formation, wherein the tumor formation is induced by the Neu oncogene.

While numerous genes that promote metastasis have previously been identified utilizing ex vivo techniques, surprisingly few have been demonstrated to be required for tumor metastases in intact animal models. Examples of such genes include Mgat5, Csf-1, CD44 and Irs-2 (Guerin et al., Oncogene Res, 3:21-31 (1988); Deming et al., Br. J. Cancer, 83:1688-1695 (2000); Van Dyke et al., Cell, 108:135-144 (2002); Woodhouse et al., Cancer, 80:1529-1537 (1997)). As shown herein for the first time, a physiological function for prediction of tumor metastasis for HUNK kinase has now been identified. Notably, the SNF1-related kinases C-TAK1 and LKB1 have previously been implicated in tumorigenesis, whereas SNRK, ARKS, and SNF1 have been implicated in the related processes of cell migration, invasion and metastasis. Consistent with these proposed functions for SNF1-related kinases, it has now been shown herein that Hunk is required in a cell-autonomous manner for the efficient metastasis of tumors. By way of a non-limiting example, Hunk is required for metastasis of c-myc-induced mammary tumors.

As described in detail elsewhere herein, Hunk knockout mice are developmentally normal, healthy, and fertile. Therefore, therapeutic intervention with Hunk activity in mice, in humans, and other mammalian systems comprising Hunk would result in minimal side effects, if any, as Hunk is not required for any essential cellular process during embryonic development, postnatal development, or adult physiology.

In an embodiment of the invention, inhibition of Hunk activity is useful to prevent metastasis of breast cancer cells. This is because it has been demonstrated for the first time herein that Hunk kinase activity is responsible for the metastatic phenotype of metastatic breast cancer cells. As set forth in detail elsewhere herein, a cell line, derived from a MMTV-myc breast cancer arising in a Hunk-knockout mouse, does not metastasize to the lungs efficiently when allowed to form a tumor in a mammary fat pad of a recipient mouse. Therefore, in one aspect of the invention, a compound that inhibits Hunk kinase activity can be used to inhibit or prevent the metastatis of a breast cancer cell. The identification, design, and use of such compounds is described in detail elsewhere herein.

In another embodiment of the invention, the expression of wild type Hunk in a non-metastatic cell line derived from a MMTV-myc breast cancer arising in a Hunk-knockout mouse restores the metastatic potential of the cell line. Conversely, the expression of a mutant Hunk, wherein the mutant Hunk lacks kinase activity, in a non-metastatic cell line derived from a MMTV-myc breast cancer arising in a Hunk-knockout mouse does not restore the metastatic potential of the cell line.

In yet another embodiment, the invention includes a method of predicting metastasis-free survival of a patient diagnosed with cancer, or a cancer-related disease or disorder. The method includes detection of a gene expression signature associated with elevated expression of HUNK, as described in detail elsewhere herein. When armed with the present disclosure, the skilled artisan will understand the methods and techniques available to obtain a HUNK gene expression signature. Cancer-related diseases and disorders for which metastasis-free survival can be predicted include, but are not limited to, cancer, a tumor, carcinoma, sarcoma, neoplasm, leukemia, lymphoma or hyperproliferative cell disease or oncogene expression.

In another aspect of the invention, a method is provided for the use of Hunk as a prognostic tool in a patient. The method includes detection of a gene expression signature associated with expression of Hunk in a patient, wherein the detected expression signature can be used to predict the behavior of a cancer-related disease or disorder in the patient. Cancer-related diseases and disorders include, but are not limited to, a tumor, cancer, carcinoma, sarcoma, neoplasm, leukemia, lymphoma or hyperproliferative cell disease or oncogene expression. In one aspect of the invention, a cancer is breast cancer. In another aspect of the invention, a method is provided for the prediction of the appropriate therapy for a patient with a cancer-related disease or disorder, for the purpose of treating the patient with the cancer-related disease or disorder.

As will be understood based on the present disclosure, one or more methods of the present invention may be combined in order to provide treatment to a patient having a cancer-related disease or is disorder. By way of a non-limiting example, the invention includes a method for the use of Hunk as a prognostic and a therapeutic-determinative tool in a patient. The method includes detection of a gene expression signature associated with expression of Hunk in a patient, wherein the detected expression signature can be used to predict the behavior of a cancer-related disease or disorder in the patient, and further, use of the expression signature and prognostic data to predict the appropriate therapy to provide to the patient.

One of the most significant challenges in the successful treatment of breast cancer is identifying those patients presenting with early stage disease who are at greatest risk for metastasis and recurrence, and who would therefore benefit most from aggressive treatment. This is particularly true for patients who present with estrogen receptor (ER)-positive, lymph node-negative tumors for whom accurately establishing prognosis is particularly problematic. The present invention meets this need. This is because the disclosure set forth herein shows for the first time that within this subset of human breast cancers, both HUNK mRNA expression and a HUNK-related expression signature can be used to accurately predict clinical outcome. As set forth in greater detail elsewhere herein, HUNK-expression is a useful tool for identifying patients with early stage disease who are at high risk for recurrence.

In an embodiment, the invention includes a method of predicting an increased rate of disease relapse in a patient diagnosed with a cancer-related disease or disorder. The method includes detection of a gene expression signature associated with elevated expression of HUNK. Cancer-related diseases and disorders useful in the method include, but are not limited to, a tumor, cancer, carcinoma, sarcoma, neoplasm, leukemia, lymphoma or hyperproliferative cell disease or oncogene expression. In an aspect of the invention, a cancer is breast cancer. In another aspect of the invention, a method of predicting an increased rate of disease relapse in a patient includes the use of the expression signature to predict the appropriate therapy to provide to the patient. Based on the disclosure set forth herein, one of skill in the art will understand how to select an appropriate course of therapy based on the expected rate of relapse for the patient.

In another embodiment of the invention, a method is provided for the use of Hunk to predict an increased rate of relapse in a patient. As described in detail elsewhere herein, a method of using Hunk includes the detection of a gene expression signature for Hunk, wherein the signature is indicative of the rate of relapse of the patient. In another aspect of the invention, a method of using Hunk to predict an increased rate of relapse in a patient also includes the use of the detected expression signature to determine the appropriate therapy for the patient.

In another embodiment of the invention, the ability of the HUNK-expression signature to predict is clinical outcome across a broad range of human breast cancers also demonstrates that HUNK can regulate pathways critical for the progression of multiple breast cancer subtypes. In one aspect of the invention, tumors bearing the HUNK signature have been identified. Such tumors include, but are not limited to, basal, HER2/neu-amplified, and luminal B breast cancer subtypes, which are overrepresented among tumors bearing the HUNK signature. Thus, the invention identifies HUNK as a target for therapeutic intervention.

Further still, in another embodiment of the invention, the determination of a HUNK expression signature can be used to diagnose a patient as having a disease or disorder related to the expression of HUNK. In one aspect, this diagnosis can subsequently be used to determine the appropriate type and amount of therapy to provide to such a patient, in order to treat, alleviate, or eliminate the HUNK-related disease or disorder. Methods of identifying such types and amounts of treatments are described in greater detail elsewhere herein.

Therefore, the present invention also includes a method of diagnosing a cancer-related disease or disorder, wherein the method comprises detection of a gene expression signature associated with elevated expression of HUNK. Cancer-related diseases and disorders that can be diagnosed using the method of the present invention include, but are not limited to, cancer, carcinoma, sarcoma, neoplasm, leukemia, lymphoma or hyperproliferative cell disease or oncogene expression in a patient. In another aspect of the invention, a method is provided for the use of Hunk to diagnose the presence of a cancer-related disease or disorder, wherein the method comprises detection of a gene expression signature associated with elevated expression of HUNK.

In an embodiment of the invention, HUNK is a useful therapeutic target for human breast cancers in a variety of clinical contexts. By way of a non-limiting example, the HUNK signature is prominently associated with highly aggressive ER-negative basal subtype tumors for which there is currently a lack of validated molecular targets. Therefore, the correlation of HUNK with this cancer illustrates that modulation of HUNK expression can be used to combat this type of cancer. Additionally, HUNK is associated with aggressive behavior within breast cancer subtypes, such as, but not limited to, ER-positive and HER2/neu-amplified tumors. The present invention particularly addresses the need to find a target by which to treat such tumors, as development of resistance to current therapeutics is common in ER-positive and HER2/neu-amplified tumors. By way of a non-limiting example, recent successes in using protein kinase inhibitors to treat BCR-ABL-positive chronic myelogenous leukemias and lung cancer bearing mutations in the epidermal growth factor receptor highlight the utility of targeting this class of molecules—which includes HUNK—pharmaceutically (Kusakai et al., J. Exp. Clin. Cancer Res., 23:263-268 (2004); Legembre et al., J. Biol. Chem., 219:46742-46747 (2004)).

Therefore, the present invention also includes a method of treating a cancer-related disease or disorder, wherein the method includes the delivery of a therapeutically-effective amount of an inhibitor of Hunk to a target cell. In one aspect, the administration of an inhibitor of Hunk is used to block the activation of HUNK in the target cell. In another aspect, the administration of an inhibitor of Hunk is used to decrease the activity of HUNK in the target cell. In yet another aspect, the administration of an inhibitor of Hunk is used to block the activation and to decrease the activity of HUNK in the target cell. Methods of determination of a “therapeutically effective amount” of an compound, as well as methods of delivery or administration of a compound to a cell, are described in detail elsewhere herein.

In one aspect of the invention, a Hunk inhibitor is a antisense molecule. In another aspect, a Hunk inhibitor is an anti-Hunk molecule, such as, but not limited to, an antibody. In yet another aspect, a Hunk inhibitor is a protein kinase inhibitor. Cancer-related diseases or disorders that can be treated using a Hunk inhibitor according to the present invention include, but are not limited to, cancer, hyperproliferative disease and oncogene expression in a patient.

The present invention is further described in the following examples. These examples are provided for purposes of illustration only, and are not intended to be limiting unless otherwise specified. The various scenarios are relevant for many practical situations, and are intended to be merely exemplary to those skilled in the art. These examples are not to be construed as limiting the scope of the appended claims. Thus, the invention should in no way be construed as being limited to the following example, but rather, should be construed to encompass any and all variations which become evident in light of the teaching provided herein.

### EXAMPLES

The screening, RNA analyses, in situ hybridization and constructions described below are carried out according to the general techniques of genetic engineering and molecular cloning detailed in, e.g., Maniatis et al., (Laboratory Manual, Cold Spring Harbor, Laboratory Press, Cold Spring Harbor, N.Y. (1989)). The steps of PCR amplification follow known protocols, as described in, e.g., PCR Protocols—A Guide to Methods and Applications (ed., Innis, Gelfand, Sninsky and White, Academic Press Inc. (1990)). Variations of such methods, so long as not substantial, are within the understanding of one of ordinary skill in the art.

### Example 1

### Protein Kinases Expressed During Mammary Development

To study the role of protein kinases in regulating mammary proliferation and differentiation, the following screen was designed to identify protein kinases expressed in the mammary gland and in breast is cancer cell lines. A reverse transcriptase (RT)-PCR cloning strategy was employed that relied on the use of degenerate oligonucleotide primers corresponding to conserved amino acid motifs present within the catalytic domain of protein tyrosine kinases (Wilks et al., Gene, 85:67-74 (1989); Wilks et al., Proc. Natl. Acad. Sci. USA, 86:1603-1607 (1989)).

### Cell Culture.

Mammary epithelial cell lines were derived from mammary tumors or hyperplastic lesions that arose in mouse mammary tumor virus (MMTV)-c-myc, MMTV-int-2, MMTV-neu/NT, or MMTV-H-ras transgenic mice and included: the neu transgene-initiated mammary tumor-derived cell lines SMF, NAF, NF639, NF1 1005, and NK-2; the c-myc transgene-initiated mammary tumor-derived cell lines 16MB9a, 8Ma1a, MBp6, M158, and M1011; the H-ras transgene-initiated mammary tumor-derived cell lines AC816, AC236, and AC711; the int-2 transgene-initiated hyperplastic cell line HB12; and the int-2 transgene-initiated mammary tumor-derived cell line 1128 (Morrison et al., 1994). Additional cell lines were obtained from ATCC and included NIH3T3 cells and the nontransformed murine mammary epithelial cell lines NMuMG and CL-S1. All cells were cultured under identical conditions in DMEM medium supplemented with 10% bovine calf serum, 2 mM L-glutamine, 100 units/ml penicillin, and 100 mg/ml streptomycin.

### Animals and Tissues.

FVB mice were housed under barrier conditions with a 12-h light/dark cycle. The mammary glands from between 10 and 40 age-matched mice were pooled for each developmental point. Mice for pregnancy points were mated at 4-5 weeks of age. Mammary gland harvest consisted in all cases of the No. 3, 4, and 5 mammary glands. The lymph node embedded in the No. 4 mammary gland was removed prior to harvest. Tissues used for RNA preparation were snap frozen on dry ice. Tissues used for in situ hybridization analysis were embedded in O.C.T. embedding medium (10.24% polyvinyl alcohol; 4.26% polyethylene glycol) and frozen in a dry ice/isopentane bath. Developmental expression patterns for 13 kinases were confirmed using independent pools of RNA. Analysis of the developmental expression pattern for an additional kinase using these independent pooled samples revealed a similar pregnancy-up-regulated expression pattern that differed with respect to the day of pregnancy at which maximal up-regulation occurred.

Construction and Analysis of Kinase-Specific cDNA Libraries.

RNA prepared from nine different sources was used as starting material for the generation of kinase-specific cDNA libraries. Kinase-specific cDNA libraries were constructed using mRNA prepared from the mammary glands of mice at specified stages of development and from a panel of mammary epithelial cell lines. Specifically, total RNA was prepared from the mammary glands of either 5-week-old nulliparous female mice or parous mice that had undergone a single pregnancy followed by 21 days of lactation and 2 days of postlactational regression. Total RNA was also prepared from the seven mammary epithelial cell lines NMuMG, CL-S1, HBI2, SMF, 16MB9a, AC816, and 1128, described above (Leder et al., Cell, 45:485-495 (1986); Muller et al., 1988; Muller et al., EMBO 1, 9:907-913 (1990); Sinn et al., Cell, 49:465-175 (1987)). Mammary tumors arising in each of these transgenic strains have previously been demonstrated to possess distinct and characteristic histopathologies that have been described as a large basophilic cell adenocarcinoma associated with the myc transgene, a small eosinophilic cell papillary carcinoma associated with the H-ras transgene, a pale intermediate cell nodular carcinoma associated with the neu transgene, and a papillary adenocarcinoma associated with the int-2 transgene (Cardiff et al., 1993; Cardiff et al., Am. J. Pathol, 139:495-501 (1991); Uvmn et al., Semin. Cancer Biol, 6:153-158 (1995)).

First-strand cDNA was generated from each of these nine sources of RNA using the cDNA Cycle kit according to the manufacturer's directions (Invitrogen, San Diego, Calif.). These were amplified using degenerate oligonucleotide primers corresponding to conserved regions in kinase catalytic subdomains VIb and IX. The degenerate primers, PTKIa (5′-GGGCCCGGATCCAC(A/C)G(A/G/C/T)GA(C/T)(C/T)-3′) SEQID NO:7, and PTKIIa (5′-CCCGGGGAATTCCA(A/T)AGGACCA(G/C)AC(G/A)TC-3′) SEQID NO:8, have previously been shown to amplify a conserved 200-bp portion of the catalytic domain of a wide variety of tyrosine kinases (Hanks et al., 1991; Wilks, 1989; Wilks, Methods Enzymol, 200:533-546 (1991)). In an effort to isolate a broad array of protein kinases, two additional degenerate oligonucleotide primers, BSTK1a (5′-GGGCCCGGATCC(G/A)T(A/G)CAC(A/C)G(A/G/C)GAC(C/T)T-3′) SEQID NO:9, and BSTK1Ia (5′ CCCGGGGAATTCC(A/G)(A/T)A(A/G)CTCCA(G/C)ACATC-3′) SEQID NO:10, were designed for use in this screen. These primers are also directed against subdomains VIb and IX, however, they differ in nucleotide sequence. Restriction sites, underlined in the primer sequences, were generated at the 5′ (Apa1 and BamHI) and 3′ (Xma1 and EcoRI) ends of the primer sequences.

Each cDNA source was amplified in three separate PCR reactions using three pairwise combinations of the PTKIa/PTKIIa, BSTKIIa/BSTKIIa, and BSTKIa/PTKIIa degenerate primers to amplify first-strand cDNA from each of the nine sources. Following 5-minutes denaturation at 95° C., samples were annealed at 37° C. for 1 min, polymerized at 63° C. for 2 min, and denatured at 95° C. for 30 s for 40 cycles. The resulting ˜200-bp PCR products were purified from low-melting agarose (Boehringer Mannheim Biochemicals, Indianapolis, Ind.), ligated into a T-vector (Invitrogen), and transformed in Escherichia coli. Following blue/white color selection, approximately 50 transformants were picked from each of the 27 PCR reactions (3 reactions for each of nine cDNA sources) and were subsequently transferred to gridded plates and replica plated. In total, 1450 transformants were analyzed. Dideoxy sequencing of 100 independent transformants was performed, resulting in the identification of 14 previously described tyrosine kinases.

In order to identify and eliminate additional isolates of these kinases from further consideration, filter lifts representing the 1350 remaining transformants were hybridized individually to radiolabeled DNA probes prepared from each of the 14 initially isolated kinases. Hybridization and washing were performed as described under final washing conditions of 0.13 SSC/0.1% SDS at 70° C. that were demonstrated to prevent cross-hybridization between kinase cDNA inserts (Marquis et al., Nat. Genet., 11:17-26 (1995). In this manner, 887 transformants (70% of the transformants) were identified that contained cDNA inserts from the 14 tyrosine kinases that had initially been isolated. Identifications made by colony hybridization were consistent with those made directly by DNA sequencing.

The remaining 463 transformants were screened by PCR using T7 and SP6 primers to identify those containing cDNA inserts of a length expected for protein kinases. One hundred seventy-two transformants were found to have cDNA inserts between 150 and 300 bp in length. These were subcloned into a plasmid vector and approximately 50 bacterial transformants from each of the 27 PCR reactions were replica plated and screened by a combination of DNA sequencing and colony lift hybridization in order to identify the protein kinase from which each subcloned catalytic domain fragment was derived.

Individual clones were sequenced using the Sequenase version 2 dideoxy chain termination kit (U.S. Biochemical Corp., Cleveland, Ohio). Putative protein kinases were identified by the DFG (aspartate-phenylalanine-glycine) consensus located in catalytic subdomain VI. DNA sequence analysis was performed using MacVector 3.5 (Oxford Molecular Group, Oxford, UK) and the NCBI BLAST server (Atschul et al., J. Mol. Biol., 215:403-410 (1990)).

### RNA Preparation and Analysis.

RNA was prepared by homogenization of snap-frozen tissue samples or tissue culture cells in guanidinium isothiocyanate supplemented with 7 ml/ml 2-mercaptoethanol, followed by ultracentrifugation through cesium chloride as previously described (Marquis et al., 1995; Rajan et al., Dev. Biol. 184, 385-401 (1997)). Poly(A)⁺ RNA was selected using oligo(dT) cellulose (Pharmacia, Piscataway, N.J.), separated on a 1.0% agarose gel (Seakem L E, BioWhittaker Molecular Applications, Rockland, Me.), and passively transferred to a Gene Screen membrane (New England Nuclear, Boston, Mass.). Northern hybridization was performed as described using ³²P-labeled cDNA probes corresponding to catalytic subdomains VI-IX of each protein kinase that were generated by PCR amplification of cloned catalytic domain fragments (Marquis et al., 1995). In all cases calculated transcript sizes were consistent with values reported in the literature. In situ Hybridization. In situ hybridization was performed as described (Marquis et al., 1995). Antisense and sense probes were synthesized with the Promega (Madison, Wis.) in vitro transcription system using ³⁵S-UTP and ³⁵S-CTP from the T7 and SP6 RNA polymerase promoters of a PCR template containing the sequences used for Northern hybridization analysis.

Analysis of the clones resulted in the identification of 33 tyrosine kinases and 8 serine/threonine kinases (Table 1). The 19 receptor tyrosine kinases and 14 cytoplasmic tyrosine kinases that were isolated accounted for all but 33 of the 1056 kinase-containing clones. The remaining clones were derived from 8 serine/threonine kinases, 7 of which were represented by a single clone each, including each of the novel kinases isolated in this screen. Approximately half of the 41 kinases were isolated more than once, and most of these were isolated from more than one tissue or cell line (Table 2 and data not shown). Eight (8) tyrosine kinases, including Jak2, Fgfr1, EphA2, Met, Igf1r, Hck, Jak1, and Neu, accounted for 830 (79%) of all clones analyzed (Table 2). Conversely, 18 kinases (44%) were represented by a single clone each, suggesting that further screening of cDNA libraries derived from these tissues and cell lines may yield additional kinases.

TABLE 2
Protein Kinases Isolated from Mammary Glands and Mammary
Epithelial Cell Lines.
Receptor tyrosine
kinases

| | |
|---|---|
| Axl/Ufo | 6 |
| EphA2 | 121 |
| EphA7 | 1 |
| EphB3 | 2 |
| Egfr | 1 |
| Fgfr1 | 126 |
| Flt3 | 1 |
| gflr | 89 |
| InsR | 1 |
| c-Kit | 2 |
| Met | 120 |
| MuSK | 1 |
| Neu | 62 |
| Ron | 10 |
| Ryk | 1 |
| Tie1 | 1 |
| Tie2 | 27 |
| Tyro10 | 2 |
| Tyro3 | 1 |

Nonreceptor tyrosine
kinases

| | |
|---|---|
| c-Abl | 5 |
| Csk | 46 |
| Ctk | 1 |
| c-Fes | 24 |
| Fyn | 7 |
| Hck | 88 |
| Jak1 | 74 |
| Jak2 | 150 |
| Lyn | 21 |
| Prkmk3 | 3 |
| c-Src | 23 |
| Srm | 1 |
| Tec | 1 |
| Tyk2 | 4 |

Serine/Threonine
kinases

| | |
|---|---|
| c-Akt1 | 1 |
| Mlk1 1 | 1 |
| Plk | 26 |
| A-Raf | 1 |
| SLK | 1 |

Novel kinases

| | |
|---|---|
| Bstk1 | 1 |
| Bstk2 | 1 |
| Bstk3 | 1 |

Note.
Kinases are arranged by family and class. The number of clones kinase is shown on the right.

Three novel protein kinases were identified in this screen, designated Bstk1, 2, and 3. Each of these kinases contains the amino acid motifs characteristic of serine/threonine kinases. Bstk2 and Bstk3 were each isolated from the mammary glands of mice undergoing early postlactational regression. Bstk1 was isolated from a mammary epithelial cell line derived from a tumor that arose in an MMTV-neu transgenic mouse, and is most closely related to the SNF1 family of serine/threonine kinases. A full-length cDNA encoding Bstk1 has subsequently been isolated and identified (Gardner et al., Genomics, 63:46-59 (2000)). Characteristics and expression patterns for the remaining 43 protein kinases isolated by this screen are reported by Chodosh et al., 2000.

### Example 2

### Cloning and Characterization of Hunk

Recognizing the unique temporal and spatial expression pattern of Bstk1, it was renamed Hunk, for hormonally-upregulated, neu-tumor-associated kinase. To isolate the full-length mRNA transcript from which Hunk (Bstk1) was derived, the initial 207-bp RT-PCR product was used to screen a murine brain cDNA library.

Isolation of cDNA Clones Encoding Hunk.

Cloning of a Full-Length Hunk cDNA. PoIy(A)⁺ RNA isolated from the MMTV-H-ras transgenic mammary epithelial tumor cell line, AC816 (Morrison, B., et al., Oncogene 9:3417-3426 (1994)), or from FVB mouse embryos harvested at day 14 of gestation, was used to generate independent cDNA libraries using either the Uni-ZAP (AC816) or the Zap Express (day 14 embryo) lambda phage vector (Stratagene, La Jolla, Calif.) according to the manufacturer's instructions. Hybridization was performed at a concentration of 10⁶ cpm/ml in 48% formamide, 10% dextran sulfate, 4.8×SSC, 20 mM Tris (pH 7.5), 1×Denhardt's solution, 20 μg/ml salmon sperm DNA, and 0.1% SDS at 42° C. overnight. Following hybridization, blots were washed in 2×SSC/0.1% SDS at room temperature (RT) for 30 minutes (×2), is followed by 0.2×SSC/0.1% SDS at 50° C. for 20 minutes (×2), and subjected to autoradiography. Positive phage clones were plaque-purified, and plasmids were liberated by in vivo excision according to the manufacturer's instructions (Stratagene).

Using standard methods and a [α-³²P]dCTP-labeled random-primed cDNA probe (Random Prime Kit, Boehringer Mannheim Biochemicals), a total of 5×10⁵ plaques were screened from each library. The murine library was screened by a cDNA probe derived from the catalytic domain fragment of Hunk, specifically from the 5′ end of the longest clone isolated, G3 (corresponding to nucleotides 618 to 824 of Hunk). The mouse embryo cDNA library was screened using cDNA fragments corresponding to nucleotides 132 to 500 and 276 to 793 of Hunk.

Six (6) additional nonchimeric cDNA clones ranging in length from 4.4 to 5.0 kb were isolated from the mouse embryo library. Each of the clones possessed a poly(A)⁺ tail and a restriction pattern similar to that of G3 (data not shown).

Sequence Analysis.

Dideoxy sequencing of the 5′ and 3′ termini of selected clones, in addition to restriction mapping revealed that all seven cDNA clones were contiguous.

Technically, sequence analyses, including predicted open reading frames and calculation of predicted molecular weights, were performed on an ABI Prism 377 DNA sequencer using Mac Vector (Oxford Molecular Group, Oxford, UK). Pairwise and multiple sequence alignments of kinase catalytic domains were performed using the ClustalW alignment program (Thompson et al., Nucleic Acids Research, 22:4673-4680, 1994) and calculations were made using the BLOSUM series (Henikoff et al., Proc Natl Acad Sci USA 89:10915-9, 1992) with an open gap penalty of 10, an extend gap penalty of 0.05, and a delay divergent of 40%. Multiple sequence alignment and phylogenetic calculations were performed using the ClustalX multisequence alignment program (Thompson et al., Nucleic Acids Research, 24:4876-4882, 1997) with the same parameters as above.

The longest cDNA clones isolated from each library, G3 and E8, were completely sequenced on both strands. Comparison of the 5024-nucleotide sequence of clone E8 with that of clone G3, revealed that clone E8 contains an additional 40 nucleotides at its 5′-end, and that the length of a poly(T) tract in the 3′-untranslated region (UTR) of the two clones differs by a single nucleotide. There were no additional differences between these two clones.

It was thus determined that clone E8 contained the entire 207-bp RT-PCR fragment, from positions 618 to 824 of Hunk (FIG. 1). The full-length Hunk cDNA sequence (FIG. 1), set forth as SEQID NO:1 (nucleic acid) and SEQID NO:2 (amino acid), respectively, have been deposited with the GenBank data-base (Accession No. AF 167987).

The finding that all six cDNA clones (isolated from a cDNA library generated from mRNA containing both 5.1- and 5.6-kb Hunk mRNA species) contained poly(A)⁺ tails and are co-linear suggested that the 5.6-kb transcript may contain additional 5′ or 3′ sequence relative to the longest cDNA clone, G3. Consistent with this understanding was the observation that insertions or deletions relative to the Hunk cDNA sequence were not detected using multiple PCR primer pairs to perform RT-PCR on first-strand cDNA prepared from RNA containing both transcripts (data not shown).

Northern Analysis of Hunk mRNA Expression

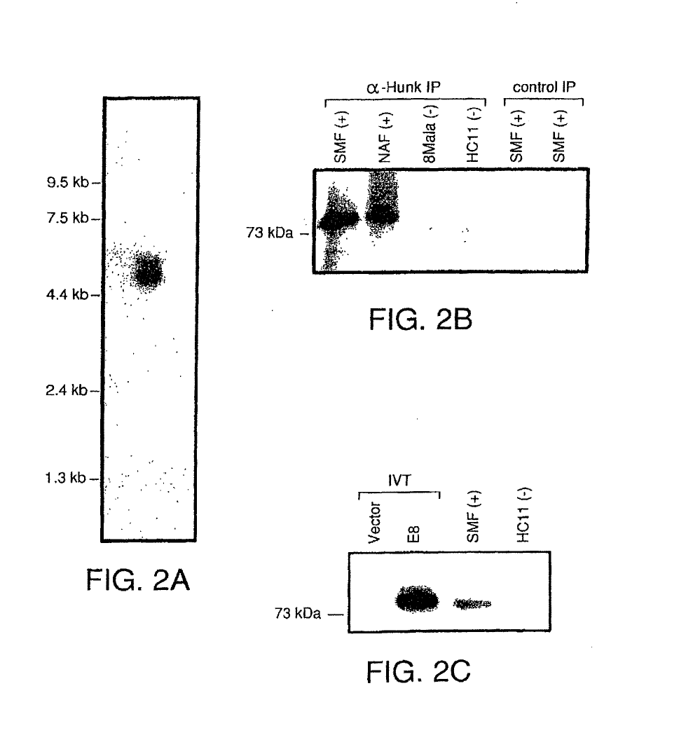

To determine whether the length of the cDNA clone encoding Hunk is consistent with the size of the Hunk mRNA message, Northern hybridization was performed on poly(A)⁺ RNA isolated from a Hunk-expressing mammary epithelial cell line (FIG. 2A). FVB mouse embryos were harvested at specified time points following timed matings. Day 0.5 postcoitus was, as above, defined as noon of the day on which a vaginal plug was observed. Tissues used for RNA preparation and protein extracts were harvested from 15- to 16-week-old virgin mice, and snap frozen on dry ice.

RNA was prepared by homogenization of snap-frozen tissue samples or tissue culture cells in guanidinium isothiocyanate supplemented with 7 μl/ml of 2-mercaptoethanol followed by ultracentrifugation through cesium chloride as reported in Example 1. 1 μg poly(A)⁺ RNA from NAF mammary epithelial cells was selected using oligo(dT) cellulose (Pharmacia), separated on a 0.7% LE agarose gel, and passively transferred to a GeneScreen membrane (NEN), again as in Example 1. Northern hybridization was performed as described using a ³²P-labeled cDNA probe encompassing nucleotides 1149 to 3849 of Hunk generated by random-primed labeling (Boehringer Mannheim Biochemicals) (Marquis et al., 1995). Hybridization was carried out as detailed above for cDNA library screening and the results are shown in FIG. 2A.

This analysis revealed a predominant mRNA transcript 5.1 kb in length in the adult tissue, as well as a less abundant transcript approximately 5.6 kb in length, suggesting that clone E8 may correspond to the shorter Hunk mRNA transcript.

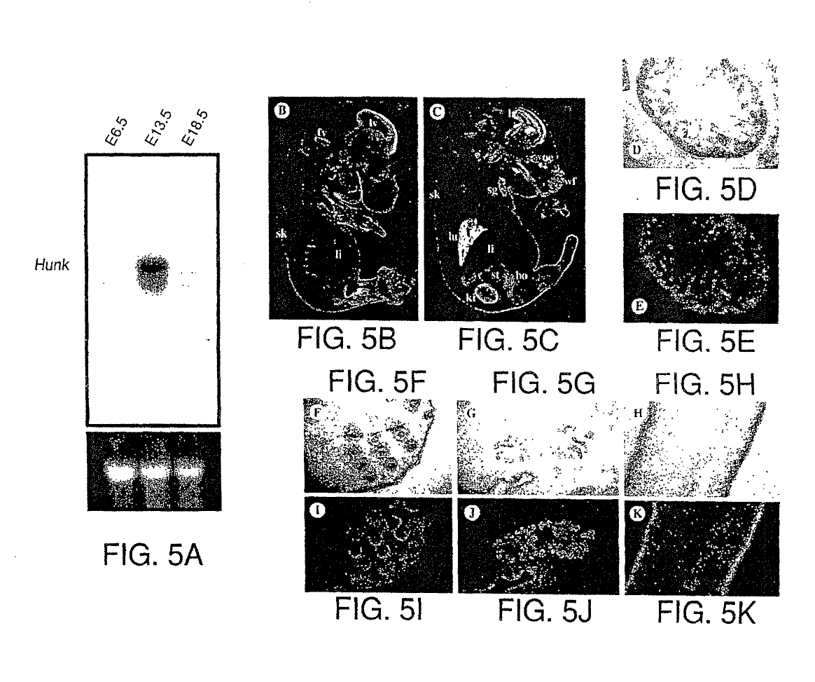

To analyze the spatial and temporal pattern of Hunk mRNA expression during fetal development, as compared with that in adult tissues, Northern hybridization analysis was performed as above, using a the generated Hunk cDNA probe on RNA isolated from FVB mice at embryonic days E6.5, E13.5, and E18.5 (2 μg poly(A)⁺ RNA samples). Hunk expression was not detected at E6.5, was dramatically up-regulated at E1 3.5, and was subsequently down-regulated at E 18.5 (FIG. 5A).

Similar to the preceding Northern analysis results obtained in adult mammary epithelial cells, analysis of embryonic mRNA revealed Hunk mRNA transcripts approximately 5.1 and 5.6 kb in length. Unlike expression in the mammary epithelial cell line, however, the 5.6-kb Hunk mRNA transcript was more abundant than the 5.1-kb transcript at E13.5, whereas the abundance of the two transcripts was equivalent at E18.5, indicating regulation of the Hunk transcripts in both a developmental stage-specific and a tissue-specific manner.

### Detection of Hunk in Mammalian Cells

Generation of Anti-Hunk Antisera.

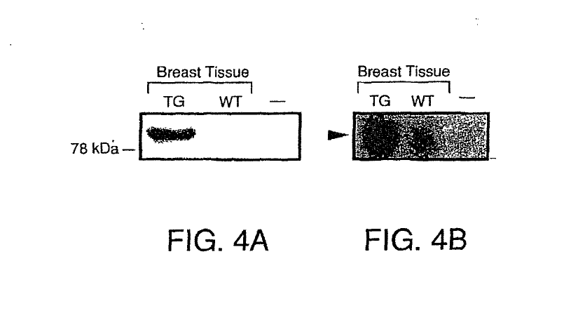

To detect the polypeptide encoded by the Hunk locus, anti-Hunk antisera were raised against recombinant proteins encoding amino-terminal (amino acids 32-213) and carboxyl-terminal (amino acids 556-714) regions of Hunk. 50 μg of protein extract prepared from mammary glands harvested from either MMTV-Hunk transgenic (TG) or wild type (WT) mice, or 100 μg of protein extract prepared from HC11 cells, a mammary epithelial cell line that does not express Hunk mRNA (−), was analyzed by immunoblotting using amino-terminal anti-Hunk antisera (FIG. 4A). FIG. 4B depicts in vitro kinase assay of Hunk immunoprecipitates. Histone H⁺ was used as an in vitro kinase substrate for Hunk protein immunoprecipitated from extracts containing 205 μg of protein, as in FIG. 2B.

GST-Hunk recombinant fusion proteins containing amino-terminal (amino acids 32-213) or carboxyl-terminal (amino acids 556-714) regions of Hunk were expressed in BL21 bacterial cells and purified using glutathione-Sepharose beads according to the manufacterer's instructions (Pharmacia). Following removal of the GST (glutathione-S-transferase) portion by cleavage with Prescission Protease (Pharmacia, Piscataway, N.J.), the liberated carboxyl-terminal Hunk polypeptide was further purified by isolation on a 15% SDS-PAGE gel.

The purified Hunk polypeptides were injected into rabbits (Cocalico Biologicals, Reamstown, Pa.) in cleavage buffer (amino-terminal) or embedded in acrylamide gel slices (carboxyl-terminal). Antisera were affinity-purified on cyanogen bromide-coupled Sepharose columns crosslinked with their respective antigens according to the manufacturer's instructions (Pharmacia). Bound antibodies were then eluted sequentially with 100 mM glycine, pH 2.5, and 100 mM triethyl-amine, pH 11.5, and neutralized with 1/10 vol of 1.0 M Tris (pH 7.5) (Harlow et al., Using Antibodies: A Laboratory Manual. Cold Spring Harbor Laboratory Press, Cold Spring Harbor, N.Y. (1999)).

Immunoprecipitation of Hunk/Immunoblotting Analysis.

To demonstrate that the identified 80-kDa polypeptide corresponded to Hunk, protein extracts prepared from two mammary epithelial cell lines that express Hunk mRNA and from two mammary epithelial cell lines that do not express Hunk mRNA were subjected to immunoprecipitation/immunoblotting protocols (FIG. 2B).

Protein was extracted from tissue culture cells by lysis in EBC buffer for 15 minutes at 4° C. From each extract, 500 μg of protein in 250 μl of EBC was precleared with 40 μl of 1:1 Protein A-Sepharose:PBS (Pharmacia, Piscataway, N.J.) for 3 hours at 4° C. Precleared lysates, prepared from cells that either express (+) or do not express (−) Hunk mRNA, were incubated overnight at 4° C. with affinity-purified antisera raised against the amino-terminus of Hunk (3 μg) (shown in FIG. 2B as α-Hunk IP), the carboxyl-terminus of Hunk (0.1 μg), or polypeptides unrelated to Hunk (0.1 or 3 μg) (shown in FIG. 2B as control IP). Immune complexes were precipitated by incubating with 40 μl of 1:1 Protein A-Sepharose:PBS for 3 hours at 4° C. Complexes were washed twice with PBS, washed once with EBC, and electrophoresed on a 10% SDS-PAGE gel.

Following transfer onto nitrocellulose membranes, immunoblotting was performed. Protein extracts were generated by lysing tissue culture cells or homogenizing murine mammary glands in EBC buffer composed of 50 mM Tris (pH 7.9), 120 mM NaCl, and 0.5% NP-40, supplemented with 1 mM β-glycerol phosphate, 50 mM NaF, 20 μg/ml aprotinin, 100 μg/ml Pefabloc (Boehringer Mannheim Biochemicals), and 10 μg/ml leupeptin. Equivalent amounts of each extract were electrophoresed on 10% SDS-PAGE gels and transferred overnight onto nitrocellulose membranes. Following visualization by Ponceau staining to verify equal protein loading and even transfer, membranes were incubated with blocking solution consisting of 4% dry milk, 0.05% Tween 20, and IX phosphate-buffered saline (PBS) at RT. Primary antibody incubation with affinity-purified antisera was performed at RT for 1 hour at a final concentration of approximately 2 μg/ml in blocking solution. Following three RT washes in blocking solution, blots were incubated with a 1:10,000 dilution of a horseradish peroxidase-conjugated goat anti-rabbit secondary antibody (Jackson ImmunoResearch, West Grove, Pa.) for 30 minutes at RT. Following three washes in blocking solution and two washes in 1×PBS, blots were developed using the ECL Plus system according to the manufacturer's instructions (Amersham Pharmacia, Piscataway, N.J.) followed by exposure to film.

As a result, after immunoprecipitation of Hunk using antisera raised against the amino-terminus of Hunk, followed by immunoblotting with antisera raised against the carboxyl-terminus of Hunk, an 80-kDa polypeptide was identified only in extracts prepared from cells that express Hunk mRNA (FIG. 2B). Similarly, immunoprecipitation of Hunk using antisera raised against the carboxyl-terminus of Hunk, followed by immunoblotting with antisera raised against the amino-terminus of Hunk, also identified an 80-kDa polypeptide only in extracts prepared from cells that express Hunk mRNA, but not in extracts from mammary epithelial cells that do not (FIG. 2C; and data not shown).

The 80-kDa polypeptide was not detected when immunoblotting was performed on immunoprecipitates prepared from Hunk-expressing cells when immunoprecipitation was carried out using either of two control affinity-purified antisera (FIG. 2B; and data not shown). This confirmed that this 80-kDa polypeptide represented the endogenous Hunk gene product in these mammary epithelial cell lines.

In Vitro Transcription/Translation.

To confirm that clone E8 encodes the predominant form of Hunk found in mammary epithelial cells, in vitro transcription and translation (IVT) of clone E8 were performed on 1 μg of plasmid DNA using rabbit reticulocyte lysates in the presence of either [³⁵S]Met or unlabeled methionine, using either plasmid control (vector) or full-length Hunk cDNA (E8) as a template, according to the manufacturer's instructions (TNT kit, Promega). Completed reactions were electrophoresed on a 10% SDS-PAGE gel along with lysates from Hunk-expressing (+) and non-expressing (−) cell lines, and were subjected either to autoradiography or to immunoblotting using antisera raised against the carboxyl-terminus of Hunk, as described below. This yielded an ˜80-kDa labeled polypeptide species, consistent with the 79.6-kDa predicted size of Hunk (data not shown), indicating that the predicted initiation codon at nucleotide 72 is capable of functioning as a translation initiation site.

Immunoblotting of protein extracts prepared from the Hunk mRNA-expressing mammary epithelial cell line SMF and from rabbit reticulocyte lysates programmed with sense RNA prepared by in vitro transcription of clone E8 identified a co-migrating 80-kDa polypeptide with the endogenous form of Hunk protein (FIG. 2C). No band was detected in reticulocyte lysates programmed with an empty vector or in whole-cell lysates from a cell line that did not express Hunk mRNA. The observation that the ˜80-kDa polypeptide identified by anti-Hunk antisera co-migrated with the polypeptide obtained following in vitro transcription and translation of clone E8 showed that it contains the entire ORF encoding the predominant form of Hunk found in mammary epithelial cells. Nevertheless, due to the absence of in-frame stop codons upstream of the putative translation initiation codon, the possibility that additional 5′ coding sequence exists cannot be excluded.

### Hunk Encodes a Functional Protein Kinase

Kinase Assays.

To demonstrate that Hunk protein levels are correlated with kinase activity, in vitro kinase assays were performed. Affinity-purified anti-Hunk antisera were used to immunoprecipitate Hunk from protein extracts prepared from the mammary glands of wild type mice, transgenic mice overexpressing Hunk, or a mammary epithelial cell line that does not express Hunk mRNA.

Transgenic mice were engineered to overexpress Hunk in the mammary gland using the mouse mammary tumor virus LTR to direct Hunk expression. Protein was extracted from snap-frozen lactating murine mammary glands and from 8Ma1a cells (Morrison et al., 1994) by dounce homogenization in EBC buffer containing protease inhibitors, as above. Extracts containing 820 μg protein in 1 ml EBC were precleared with 40 μl 1:1 Protein A-Sepharose:PBS (Pharmacia) for 1 hour at 4° C. One-quarter of the precleared lysate was incubated at 4° C. overnight with 1.2 μg/ml of affinity-purified anti-sera raised against the amino-terminus and carboxyl-terminal of Hunk were used in immunoblotting experiments to detect Hunk in protein extracts prepared from the mammary glands of wildtype mice or MMTV-Hunk transgenic mice harvested at day 9 of lactation (FIG. 4A).

Immune complexes were precipitated with 40 μl of 1:1 Protein A-Sepharose:PBS. In vitro kinase activity of the resulting immunoprecipitates was assayed by incubated with [γ-³²P]ATP and either histone H1 or myelin basic protein as substrates (FIG. 4B; and data not shown). The final reaction conditions consisted of 20 mM Tris (pH 7.5), 5 mM MgCl2, 100 μM dATP, 0.5 μCi/ml [γ-³²P]ATP, and 0.15 μg/μl histone H1 for 45 minutes at 37° C. Reactions were electrophoresed on a 15% SDS-PAGE gel, and were subjected to autoradiography.

Hunk immunoprecipitates were able to phosphorylate both histone H1 and MBP in vitro. As predicted based on the relative quantities of Hunk immunoprecipitated from transgenic and wild type mammary glands (data not shown), Hunk-associated phosphotransferase activity was substantially greater in immunoprecipitates prepared from transgenic compared to wildtype mammary glands. No activity was observed in immunoprecipitates prepared from a cell line known not to express Hunk mRNA. Thus, these findings demonstrate that anti-Hunk antisera co-immunoprecipitate Hunk and a phosphotransferase, further confirming that Hunk encodes a functional protein kinase.

RNase Protection Analysis.

FIG. 5 A depicts an RNase protection analysis of Hunk mRNA spacial expression in tissues of the adult mouse. 30 μg of RNA isolated from the indicated murine tissues was hybridized with antisense RNA probes specific for Hunk and for β-actin. Ribonuclease protection analysis was performed as described (Marquis et al., 1995). Body-labeled anti-sense riboprobes were generated using linearized plasmids containing nucleotides 276 to 500 of the Hunk cDNA and 1142 to 1241 of β-actin (GenBank Accession No. X03672) using [α-³²P]UTP and the Promega in vitro transcription system with T7 polymerase. The β-actin antisense riboprobe was added to each reaction as an internal control. Probes were hybridized with RNA samples at 58° C. overnight in 50% formamide/100 mM Pipes (pH 6.7). Hybridized samples were digested with RNase A and T1, purified, electrophoresed on a 6% denaturing polyacrylamide gel, and subjected to autoradiography.

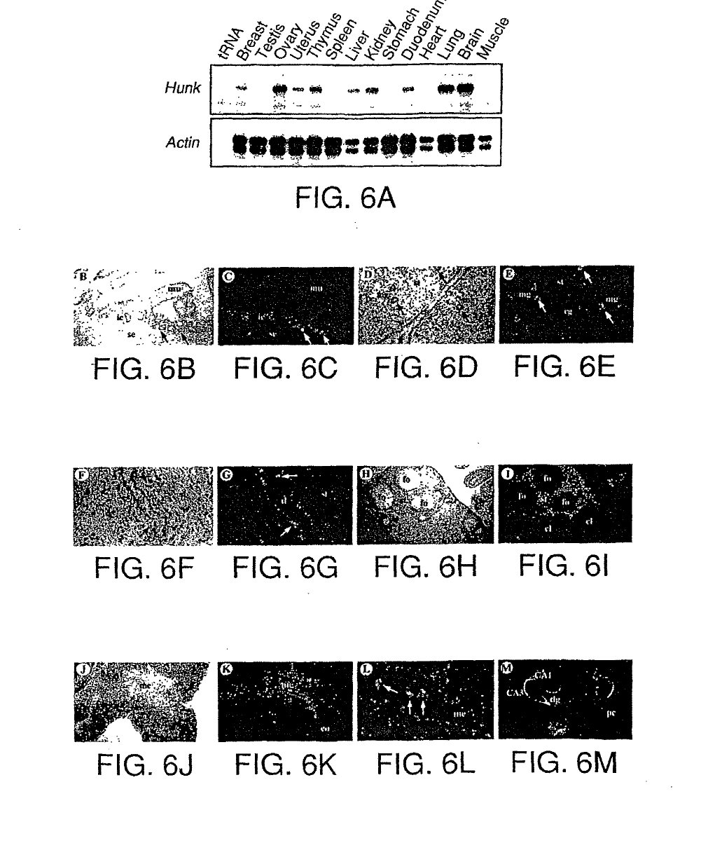

The spacial distribution of Hunk is summarized in adult tissue in FIG. 6A. High levels of Hunk expression were detected in ovary, thymus, lung, and brain, with modest levels of expression in breast, uterus, liver, kidney, and duodenum. Hunk mRNA expression was very low or undetectable in heart, skeletal muSci.e, testis, spleen, and stomach.

In Situ Hybridization.

To determine the spatial localization of Hunk mRNA expression during fetal development, ³⁵S-labeled anti-sense probes were used to perform in situ hybridization on E13.5 and E18.5 embryos (FIGS. 5B-5K). In situ hybridization was performed on FVB embryo tissue sections 15- to 16-week-old virgin mice embedded in OCT compound (as described by Marquis et al., 1995), hybridized with a ³⁵S-lableled Hunk antisense cDNA probe, see Northern analysis above. Antisense and sense probes were synthesized with the Promega in vitro transcription system using ³⁵S-UTP and ³⁵S-CTP from the T7 and SP6 RNA polymerase promoters of a PCR template containing sequences corresponding to nucleotides 276 to 793 of Hunk, a region demonstrated to recognize both mRNA transcripts. Exposure times were 6 weeks in all cases. No signal over background was detected in serial sections hybridized with sense Hunk probes to bowel, fourth ventricle, kidney, liver, lung, lateral ventricle, olfactory epithelium, submandibular gland, skin, stomach, and whisker hair follicle.

Interestingly, Hunk was shown to be expressed in only a subset of cells within each expressing organ. In the duodenum, Hunk is expressed in a subset of epithelial cells located in duodenal crypts, whereas little or no expression is observed in more differentiated epithelial cells of the duodenum or in the mesenchymal compartment of this tissue (FIGS. 6B and 6C). Heterogeneity was also observed among the crypt cells themselves, whereby cells expressing Hunk mRNA at high levels are located adjacent to cells expressing Hunk at substantially lower levels.

Heterogeneous expression patterns were also observed in other tissues. For instance, Hunk mRNA expression in the uterus is restricted to isolated epithelial cells located in mesometrial glands (FIGS. 6D and 6E). Similarly, Hunk expression in the prostate is found within only a subset of ductal epithelial cells (FIGS. 6F and 6G). Hunk expression in the ovary is found principally in the stroma, with little or no expression detected in developing follicles or corpora lutea (FIGS. 6H and 6I). Hunk expression in the thymus is limited primarily to the thymic medulla with lower levels of expression in the thymic capsule (FIGS. 6J and 6K).

Hunk is expressed throughout the brain, with particularly high levels at E13.5 in the cortex, dentate gyrus, and CA1 and CA3 regions of the hippocampus (FIG. 6M), skin, and developing bone, as well as more diffuse expression throughout the embryo. High-power examination also revealed marked heterogeneity in Hunk expression among different cell types in the cerebral cortex (data not shown). As in other tissues, expression in the thymic medulla was markedly heterogeneous (FIG. 5L). Expression of Hunk was more restricted at E1 8.5, with particularly prominent hybridization in the brain, lung, salivary gland, olfactory epithelium, skin, whisker hair follicles, and kidney. Thus, Hunk is expressed in a variety of tissues of the adult mouse, and expression within these tissues is generally restricted to a subset of cells within a particular compartment or compartments.

Chromosomal Localization; Interspecific Mouse Backcross Mapping.

The mouse chromosomal location of Hunk was determined by interspecific backcross analysis using progeny derived from matings of [(C57BL/6J×M. spretus)F₁ female×C57BL/6J male] mice, as described (Copeland et al., Trends Genet. 7:113-118 (1991)). A total of 205 N₂ mice were used to map the Hunk locus (see details below).

DNA isolation, restriction enzyme digestion, agarose gel electrophoresis, Southern transfer, and hybridization were performed essentially as described (Jenkins et al., J. Virol. 43:26 (1982)). A 520-bp EcoRI fragment corresponding to nucleotides 276 to 793 of the Hunk cDNA was labeled with [α-³²P]dCTP using a nick-translation labeling kit (Boehringer Mannheim Biochemicals). Washing was performed at a final stringency of 1.0 SSCP/0.1% SDS at 65° C. All blots were prepared with Hybond-N⁺ nylon membrane (Amersham, Arlington Heights, Ill.).

A major fragment of 6.9 kb was detected in Sacl-digested C57BL/6J DNA, and a major fragment of 5.8 kb was detected in Sacl-digested M. spretus DNA. The presence or absence of the 5.8-kb Sacl M. spretus-specific fragment was followed in backcross mice. A description of the probes and RPLPs for the loci linked to Hunk, including App, Tiam1, and Erg has been reported previously (Fan et al., Mol. Cell. Neurosci., 7:519 (1996)). Recombination distances were calculated using Map Manager, version 2.6.5 (Manly et al., Mammalian Genome, 4:303-313 (1993). Gene order was determined by minimizing the number of recombination events required to explain the allele distribution patterns.

This interspecific backcross mapping panel has been typed for over 2800 loci that are well distributed among all the autosomes as well as the X chromosome (Copeland et al., Trends Genet., 7:113-118 (1991)). C57BL/6J and M. spretus DNA samples were digested with several enzymes and analyzed by Southern blot hybridization for informative restriction fragment length polymorphisms (RFLPs) using a mouse Hunk cDNA probe.

The 5.8-kb Sacl M. spretus RFLP was used to follow the segregation of the Hunk locus in backcross mice. The mapping results indicated that Hunk is located in the distal region of mouse chromosome 16 linked to App, Tiam1, and Erg. Although 104 mice were analyzed for every marker and were evaluated by a segregation analysis (not shown), up to 152 mice were typed for some pairs of markers. Each locus was analyzed in pair-wise combinations for recombination frequencies using the additional data. The ratios of the total number of mice exhibiting recombinant chromosomes to the total number of mice analyzed for each pair of loci and the most likely gene order are: centromere-, App-4/123-Hunk-0/130-Tiam1-4/1 52-Erg. The recombination frequencies (expressed as genetic distances in centimorgans (cM)±the standard error) are −App-3.3±1.6 (Hunk, Tiam1)-2.6±1.3-Erg.

No recombinants were detected between Hunk and Tiam1 in 130 animals typed in common, suggesting that the two loci are within 2.3 cM of each other (upper 95% confidence limit). When the interspecific map of chromosome 16 was compared with a composite mouse linkage map that reports the map location of many uncloned mouse mutations (at http://www.informatics.jax.org/), Hunk mapped in a region of the composite map that lacks mouse mutations (data not shown).

### Example 3

### Developmental Role of Hunk Kinase in Pregnancy-Induced Changes in the Mammary Gland

Animal and Tissue Preparation

FVB mice were housed under barrier conditions with a 12-hour light/dark cycle. Mammary glands from pregnant females were harvested at specified time points after timed matings. Day 0.5 was defined as noon of the day on which a vaginal plug was observed. Gestational stage was confirmed by analysis of embryos. Transgenic mothers were housed with wild type mothers immediately after parturition to ensure pup survival and equivalent suckling stimuli. Both transgenic and wild type females were observed to nurse pups.

For experiments involving chronic hormone treatment, adult female FVB mice were subject to bilateral oophorectomy and allowed to recover for two weeks prior to hormonal injections that were administered as previously described (Marquis et al., 1995). For short-term hormone administration experiments, four-month-old virgin female FVB mice were injected subcutaneously with either phosphate buffered saline (PBS) or a combination of 5 mg progesterone in 5% gum arabic and 20 μg of 17β-estradiol in PBS. Four animals from each treatment group were sacrificed 24±1 hours after injection.

Tissues used for RNA analysis were snap frozen on dry ice. Tissues used for in situ hybridization analysis were embedded in OCT compound. For whole mount analysis, number four mammary glands were spread on glass slides and fixed for 24 hours in 10% neutral buffered formalin. Glands were subsequently immersed in 70% ethanol for 15 minutes followed by 15 minutes in deionized water prior to staining in 0.05% Carmine/0.12% aluminum potassium sulfate for 24-48 hours. Glands were dehydrated sequentially in 70%, 90% and 100% ethanol for 10 minutes each, and then cleared in toluene or methyl salicylate overnight.

For histological analysis, mammary glands were fixed as above, and transferred to 70% ethanol prior to paraffin embedding. Sections 5 μm thick were cut and stained with Hematoxylin and Eosin. For BrdU (5-bromodeoxyuridine) analysis of cellular proliferation (Cells: A Laboratory Manual. D. Spector, R. Goldman and L. Leinwand eds. Cold Spring Harbor Laboratory Press, 1998)., animals were injected with 50 μg BrdU per g total bodyweight two hours before sacrifice followed by fixation and paraffin embedding as above. Generation of MMTV-Hunk transgenic mice.

A full-length cDNA clone, G3, encoding Hunk, was digested with maI and SpeI to liberate a 3.2 kb fragment containing the complete coding sequence for Hunk (GenBank Accession number AF167987). This fragment was cloned downstream of the mouse mammary tumor virus long terminal repeat (MMTV LTR) into the multiple cloning site of pBS-MMTV-pA (Gunther, unpublished), which consists of the MMTV LTR upstream of the H-ras leader sequence (Huang et al., Cell, 27:245-255 (1981)) and SV40 splicing and polyadenylation signals. Linearized plasmid DNA was injected into fertilized oocytes harvested from superovulated FVB mice.

Tail-derived DNA was prepared as described (Hogan et al., Cold Spring Harbor, N.Y.: Cold Spring Harbor Laboratory Press (1994)). Mice were genotyped by Southern hybridization analysis and by two independent PCR reactions designed to amplify a region within the SV40 portion of the transgene, and a region spanning the junction between Hunk and SV40 sequences. A portion of the Gapdh (glycelaldehyde-3-phosphate dehydrogenase) locus (GenBank accession No. M32599) was amplified as a positive control for PCR reactions. Oligonucleotide primer sequences were Gapdh F:

### CTCACTCAAGATTGTCAGCAATGC (SEQID NO:11); Gapdh B:

### AGGGTTTCTTACTCCTTGGAGGC (SEQID NO:12); SV40 F:

### CCTTAAACGCCTGGTGCTACGC (SEQID NO:13); SV40 B:

### GCAGTAGCCTCATCATCACTAGATGG (SEQID NO:14); Hunk F:

CTTTCTTTTTCCCCTGACC (SEQID NO:15); PolyA⁺ B: ACGGTGAGTAGCGTCACG (SEQID NO:16). Southern hybridization analysis of tail-derived genomic DNA digested with SpeI was performed according to standard methods using a probe specific to the SV40 portion of the transgene.

Four founder mice were identified harboring the MMTV-Hunk transgenein tail-derived DNA that passed the transgene to offspring in a Mendelian fashion. These were screened for transgene expression by Northern hybridization and RNase protection analysis. One founder line, MHK3, was identified that expressed the MMTV-Hunk transgene at high levels. Of note, a subset of transgene-positive MHK3 animals was found not to express the MMTW-Hunk transgene. All MHK3 non-expressing animals were analyzed by Southern hybridization analysis to confirm transgene presence and the expected MHK3-specific integration site.

The tightly regulated expression of Hunk observed in the mammary gland during pregnancy and in response to ovarian hormones indicates that Hunk may play a role in mediating pregnancy-induced changes in the mammary gland. To test this hypothesis, transgenic mice overexpressing Hunk in a mammary-specific fashion were generated using the MMTV LTR. Activity of the MMTV LTR was up-regulated in mammary epithelial cells during pregnancy and lactation in response to rising levels of prolactin, progesterone and glucocorticoids.

Since endogenous Hunk expression is heterogeneous and transiently up-regulated during early pregnancy, MMTV-driven expression of Hunk in transgenic mice was predicted to alter the temporal and spatial profile of Hunk expression in the mammary gland. Accordingly, a cDNA encoding the full-length Hunk protein was cloned downstream of the MMTV LTR and injected into superovulated FVB mice.

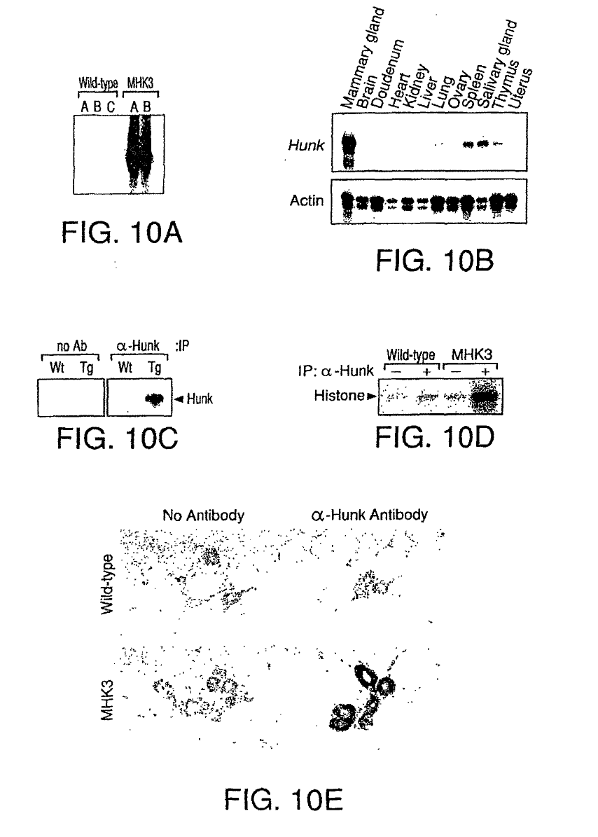

One of the four founder lines, MHK3, was found to express the Hunk transgene at high levels in the mammary gland and was, therefore, studied further (FIG. 10A). The tissue specificity of transgene expression in the MHK3 line was determined by RNase protection analysis, as described in greater detail below, using a transgene-specific probe (FIG. 10B). This analysis confirmed that nulliparous MHK3 transgenic females express high levels of the MMTV-Hunk transgene in the mammary gland and lower but detectable levels of transgene expression in the spleen, salivary gland, lung and thymus, as has been observed for other MMTV transgenic mouse models.

The hormonally responsive nature of the MMTV LTR often results in low levels of expression in the mammary glands of nulliparous transgenic animals and high levels of transgene expression during pregnancy that peak during lactation. In contrast, MHK3 animals express high levels of the MMTV-Hunk transgene in the nulliparous state. In addition, MMTV-Hunk transgene expression levels in mammary glands from pregnant or lactating MHK3 animals were found to vary less than 3-fold relative to nulliparous MHK3 animals, a range of expression that is far less than that typically found in MMTV-based transgenic mouse models (data not shown). Together, these data indicate that MMTV-Hunk transgene expression is high relative to endogenous Hunk expression during all stages of postnatal mammary development.

To determine if Hunk mRNA levels in transgenic mice resulted in changes in Hunk protein levels, antisera specific to Hunk were used to analyze Hunk expression levels in extracts prepared from lactating mammary glands of MHK3 transgenic and wild type mice (FIG. 10 C).

Protein Analysis.

Generation of anti-Hunk antisera, immunoblotting and immunoprecipitation were performed, as described in the previous examples. Protein was extracted from mammary glands by dounce homogenization in EBC buffer, also as described in the previous examples. For immunoprecipitation, 500 μg of protein (3 mg/ml) was precleared with 1/10 vol of 1:1 protein A-Sepharose in PBS overnight at 4° C. Precleared lysates were incubated overnight at 4° C. in EBC (50 mM Tris-HCl, pH 7.9; 120 mM NaCl; 0.5% NP40), plus 5% Tween 20 (Bio-Rad, Hercules, Calif.) with or without affinity-purified antisera raised against the C-terminus of Hunk (0.4 μg/ml). Immune complexes were precipitated by incubating with 40 μl of 1:1 protein A-Sepharose in PBS for 1 hour at 4° C. Complexes were washed sequentially with EBC plus 5% Tween 20, EBC (2×), and PBS (2×).

One-fifth of the precipitated complexes were used in an in vitro kinase reaction as previously described in the preceding examples, with 5 μM ATP and 0.5 μg/ml histone H1. The remaining precipitate was electrophoresed on a 10% SDS-PAGE gel, transferred onto a PVDF membrane, and immunoblotted with an antibody against the C-terminus of Hunk, also as described in the preceding to examples.

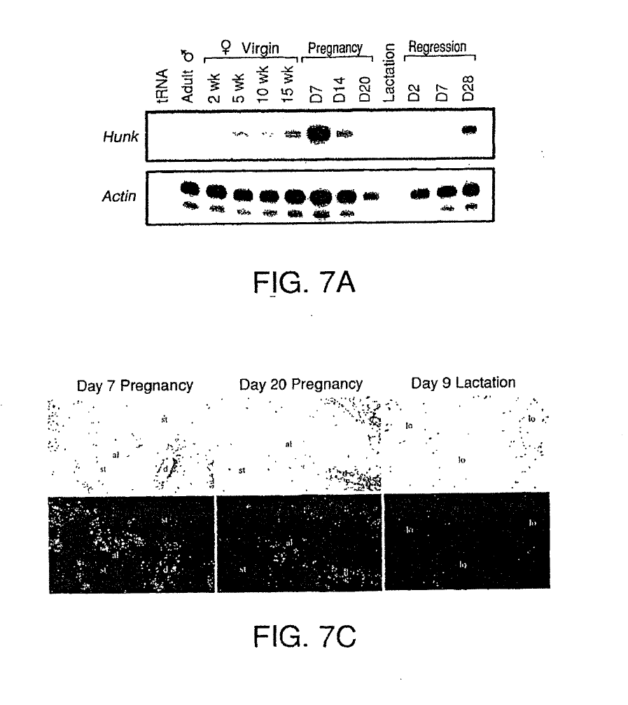

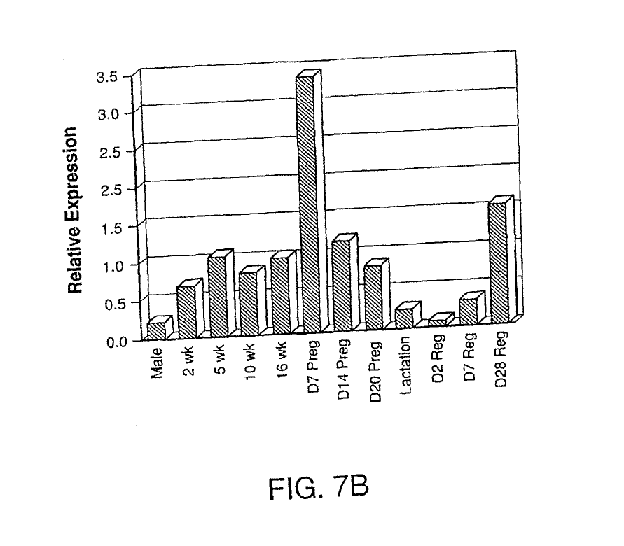

Western analysis of immunoprecipitated Hunk using Hunk-specific antisera revealed increased amounts of Hunk protein in extracts prepared from transgenic when compared with wild type mammary glands (FIG. 10C). The inability to detect Hunk protein in extracts from wild type lactating glands was consistent with the barely detectable levels of endogenous Hunk mRNA expression during this developmental stage (FIG. 7). Conversely, MMTV-Hunk transgene expression was very high during lactation (data not shown).

Hunk-Associated Kinase Activity.

To demonstrate that Hunk-associated kinase activity is also elevated in MHK3 transgenic animals, in vitro kinase assays were performed. Hunk was immunoprecipitated from protein extracts prepared from the lactating mammary glands of wild type or transgenic mice as above (FIG. 10D). Control immunoprecipitation reactions were carried out in the absence of anti-Hunk antisera. The resulting immunoprecipitates were incubated with γ-³²P-ATP and histone H1.

As predicted, based on the relative quantities of Hunk in these extracts, Hunk-associated kinase activity was substantially greater in immunoprecipitates prepared from transgenic when compared with wild type mammary glands, confirming that MHK3 transgenic animals manifest increased levels of both Hunk protein and Hunk-associated kinase activity.

Immunohistochemistry.

To investigate the spatial pattern of Hunk protein expression in MHK3 transgenic animals, immunohistochemistry was performed on mammary glands harvested from 14-week old nulliparous transgenic and wild type female mice (FIG. 10E). Mammary glands from nulliparous wild type and MHK3 transgenic females were fixed in 4% paraformaldehyde overnight and transferred to 70% ethanol prior to paraffin embedding. 5 μm sections were dewaxed in xylene and sequentially rehydrated in 100%, 95% and 70% ethanol, followed by PBS. Sections were incubated in Antigen Unmasking Solution (Vector Laboratories, Burlingame, Calif.) for 30 minutes at 100° C., and then transferred to PBS at room temperature (RT). Sections were incubated for 2 hours at RT with antibody raised against the C-terminus of Hunk, washed in PBS (×3), then incubated with 1:500 biotinylated goat anti-rabbit antibody (Vector Laboratories) in 1% BSA/PBS for 30 minutes at RT. After washing in PBS (×3), slides were incubated in a 1:250 dilution of Avidin (Vector Laboratories) for 15 minutes at RT and washed in PBS (3×). NBT and BCIP substrate addition was performed in alkaline phosphate buffer for 3 minutes according to manufacturer instructions (Boehringer Mannheim Biochemicals). Sections were counter-stained for 10 minutes in 0.5% (w/v) Methyl Green in 1.0 M NaOAc (sodium acetate), pH 4.0.

Consistent with high levels of MMTV-Hunk mRNA expression in MHK3 mice, this analysis revealed high levels of Hunk protein expression in transgenic, as compared with wild type mammary glands. As described for other MMTV transgenic models, exogenously expressed Hunk was restricted to the epithelium of MHK3 mice. In addition, Hunk expression in the mammary epithelium of MHK3 animals was found to be relatively homogeneous, unlike the heterogeneous patterns of transgene expression observed in other MMTV transgenic models or the heterogeneous expression of endogenous Hunk mRNA. These data indicate that as compared with wild type animals, MHK3 transgenic animals overexpress Hunk in a mammary epithelial-specific and relatively homogenous manner.

Notably, some MHK3 transgenic animals did not express the MMTV-Hunk transgene. The presence of the MHK3-specific transgene integration site was confirmed by Southern hybridization analysis for all non-expressing MHK3 transgenic mice. A similar type of transgene silencing has been observed in other MMTV transgenic models (Betzl et al., Biol. Chem., 377:711-719 (1996); Sternlicht et al., Cell, 98:137-146 (1999)).

### Hunk Expression is Developmentally Regulated in the Mammary Gland.

RNase Protection Analysis.

RNase protection analysis was used to determine the temporal pattern of Hunk expression during the postnatal development of the murine mammary gland (FIG. 7A), and to distinguish transgenic from endogenous Hunk expression in MHK3 animals. Mammary glands were harvested from male FVB mice, virgin mice at developmental time points prior to puberty (2 weeks), during puberty (5 weeks) and after puberty (10 weeks and 15 weeks), as well as from mice during early, mid and late pregnancy (day 7, 14 and 20), lactation (day 9), and during postlactational regression (days 2, 7 and 28).

Ribonuclease protection analysis was performed, as described in Example 2, using 40 pig samples of total RNA isolated from mammary glands at the indicated time points in FIG. 7A, hybridized to a ³²P-labeled antisense RNA riboprobe spanning the 3′-end of the Hunk cDNA, specific to nucleotides 276-500 of Hunk, the 5′-end of the SV40 polyadenylation signal sequence, and nucleotides 1142-1241 of β-actin added to each reaction as an internal control (GenBank Accession number X03672). RNA preparation, Northern hybridization and labeling of cDNA probes were performed (as described in the previous Examples; Marquis et al., 1995). The ³²P-labeled cDNA probe for Hunk encompassed nucleotides 275 to 793 (GenBank Accession number AF167987) Signal intensities were quantified by phosphorimager analysis (Molecular Dynamics, Sunnyvale, Calif.).

Steady-state levels of Hunk mRNA were shown to be low, and remained relatively constant throughout virgin development. During early pregnancy (day 7), when alveolar buds begin to proliferate rapidly and differentiate, Hunk mRNA levels underwent a dramatic increase and then returned to baseline by mid-pregnancy (FIG. 7A, 7B). The apparent decline in β-actin expression seen by RNase protection analysis during late pregnancy, lactation and early postlactational regression results from a dilutional effect that is due to large-scale expression of genes for milk proteins during late pregnancy and lactation (Buhler et al., Dev. Biol., 155:87-96 (1993); Gavin et al., Mol. Cell. Biol., 12:2418-2423 (1992); Marquis et al., 1995). Quantification and normalized of Hunk expression to β-actin to control for this dilutional effect confirmed that Hunk expression returned to baseline levels by mid-pregnancy, and decreased further during lactation and early postlactational regression (FIG. 7B).

An essentially identical expression profile was observed during pregnancy when Hunk mRNA levels were normalized to cytokeratin 18, an epithelial-specific marker, indicating that developmental changes in Hunk expression are not the result of changes in epithelial cell content in the gland during pregnancy (data not shown).

This conclusion was supported by the finding that Hunk mRNA expression levels decreased from day 7 to day 14 of pregnancy, despite ongoing increases in epithelial cell content that occur during this stage of development. Furthermore, changes in Hunk expression did not appear to be the result of increased cellular proliferation, since the pattern of Hunk expression observed during pregnancy did not correlate with levels of epithelial proliferation that, unlike Hunk expression, remained elevated during mid-pregnancy as graphically shown in FIG. 11B.

In Situ Hybridization.

In order to determine whether the observed pregnancy-induced changes in Hunk mRNA expression levels represent global changes in expression throughout the mammary gland, or changes in expressing subpopulations of cells, in situ hybridization was performed (FIG. 7C, and data not shown), as described in Example 2, using a PCR template containing nucleotides 276 to 793 of Hunk at the time points shown in FIG. 7C. Exposure times were 6 weeks in all cases.

Consistent with the results from RNase protection analysis, in situ hybridization confirmed that Hunk expression in the mammary gland was highest at day 7 of pregnancy and decreased progressively throughout the remainder of pregnancy and lactation. This analysis also revealed that Hunk was expressed exclusively in the epithelium throughout mammary gland development, and that Hunk up-regulation during pregnancy appeared to result from both the up-regulation of Hunk in a subset of cells and an increase in the proportion of Hunk-expressing epithelial cells (FIG. 7C, and data not shown).

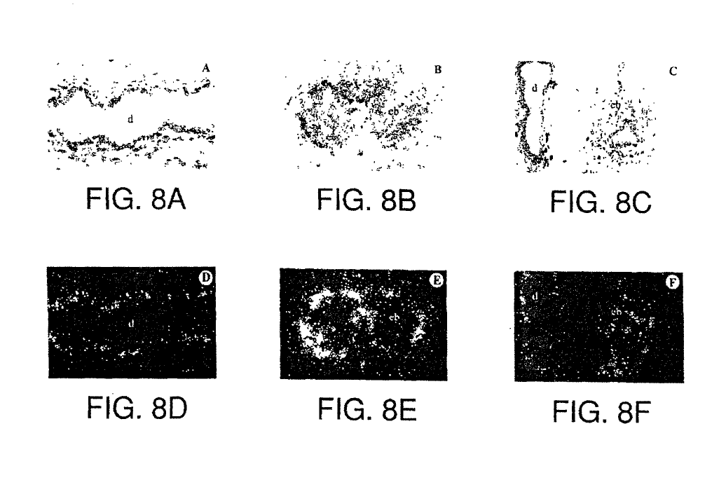

The observation that cells that express Hunk at a high level, are found adjacent to non-expressing cells, indicates that, as in other organs of the adult mouse, Hunk expression in the mammary gland is spatially restricted (FIG. 7C and FIG. 8). This heterogeneous expression pattern is particularly striking in terminal end buds and epithelial ducts of the adolescent gland (FIG. 8), indicating that the murine mammary epithelium is composed of Hunk-expressing and Hunk non-expressing cell types.

### Hunk Expression is Regulated by Ovarian Hormones

The observation that Hunk mRNA levels increase in the mammary gland during pregnancy, led to an analysis of whether expression of Hunk is modulated by estrogen and progesterone. Oophorectomized FVB 5-week-old nulliparous female mice were treated for fourteen (14) days with 17β-estradiol alone, progesterone alone, or a combination of both hormones. Intact (sham) and oophorectomized, non-hormone treated (OVX) animals were used for comparison.

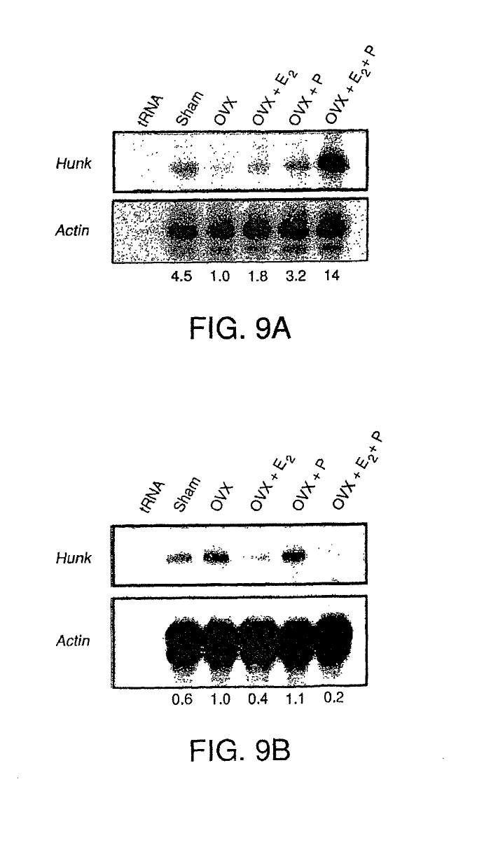

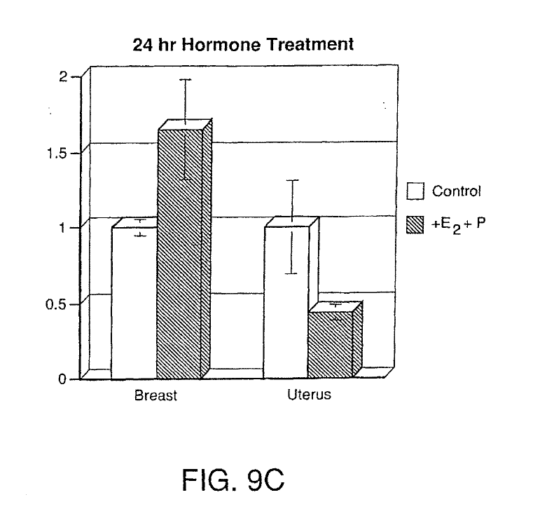

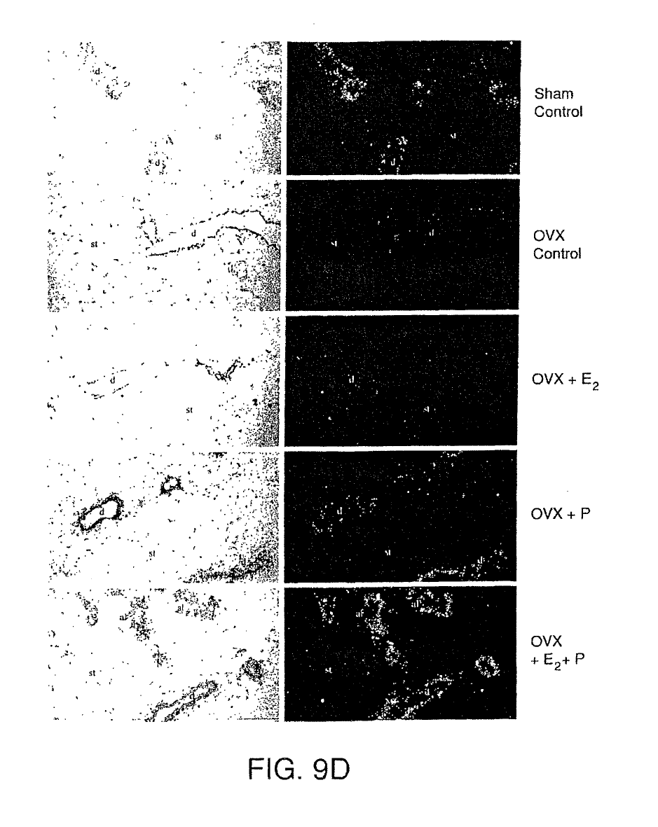

Hunk mRNA levels were quantified by RNase protection analysis, as described above, of samples of total RNA prepared from mammary glands (20 μg) (FIG. 9A) or uteri (40 μg) (FIG. 9B) pooled from at least 10 animals in each experimental group. Hybridization was performed overnight with ³²P-Iabeled antisense RNA probes specific for Hunk and β-actin. Signal intensities were quantified by phosphorimager analysis, and Hunk expression was normalized to β-actin expression levels. Steady-state Hunk mRNA levels were found to be approximately 4-fold lower in the mammary glands of oophorectomized mice when compared with intact mice, indicating that maintenance of basal levels of Hunk expression in the mammary glands of nulliparous mice requires ovarian hormones (FIG. 9A).

Treatment of oophorectomized animals with 17β-estradiol alone, increased Hunk mRNA expression. But the increase was only to levels below those observed in intact animals. By comparison, treatment with progesterone alone, increased Hunk mRNA expression to levels comparable with those observed in intact animals. In contrast, treatment of oophorectomized animals with both 17β-estradiol and progesterone resulted in a 14-fold increase in the level of Hunk mRNA relative to control oophorectomized animals, and a 3-fold increase relative to intact animals, similar to increases in Hunk expression observed during early pregnancy. These observations indicated that the increase in Hunk mRNA expression observed in the mammary gland during early pregnancy results, either directly or indirectly, from increases in circulating levels of steroid hormones, such as estrogens and progesterone.

Treatment of mice with ovarian hormones also affected Hunk expression in the uterus (FIG. 9B). Steady-state Hunk mRNA levels were nearly 2-fold higher in oophorectomized animals compared with intact mice suggesting that circulating levels of 17β-estradiol may repress Hunk expression in the uteri of nulliparous mice. Consistent with this suggestion, treatment of oophorectomized animals with 17β-estradiol, either alone or in combination with progesterone, decreased Hunk expression to levels below those observed in either intact or oophorectomized animals.

In contrast to findings in the mammary gland, progesterone treatment had little if any effect on Hunk expression in the uterus. These results indicated that the increase in Hunk mRNA expression observed in the uterus following oophorectomy is due, either directly or indirectly, to loss of tonic inhibition of Hunk expression by estradiol. The observation that the combination of estradiol and progesterone has opposing effects on Hunk expression in the mammary gland and uterus is consistent with the opposing physiological effects of these hormones on proliferation and differentiation in these tissues.

The effects of estradiol and progesterone on Hunk expression in the mammary gland and uterus were confirmed by in situ hybridization analysis performed on tissues from the experimental animals described above (FIG. 9D, and data not shown). Consistent with RNase protection results, oophorectomy resulted in a marked decrease in Hunk mRNA expression in the mammary epithelium and the combination of 17β-estradiol and progesterone resulted in a synergistic increase in Hunk expression. Reminiscent of Hunk expression during early pregnancy, the up-regulation of Hunk mKNA levels in oophorectomized animals treated with a combination of 17β-estradiol and progesterone occurred in a subset of epithelial cells in both ducts and developing alveolar buds.

Since the above experiments involved the chronic administration of hormones, sufficient time elapsed during hormone treatment for significant developmental changes to occur in both the mammary glands and uteri of oophorectomized animals. As such, these experiments do not distinguish whether changes in Hunk expression reflect direct regulation by ovarian hormones, or are a consequence of the changes in epithelial proliferation and differentiation that occur in response to the chronic administration of ovarian hormones.

To address this issue, mice were treated with 17β-estradiol, progesterone, or a combination of 5 mg progesterone in 5% gum arabic and 20 μg of 17β-estradiol in PBS. Injection with PBS alone was used as control (FIG. 9C). Analysis of Hunk mRNA expression levels in these mice revealed a pattern similar to that observed in mice treated chronically with hormones. Within 24 hours of the administration of 17β-estradiol and progesterone, steady-state levels of Hunk mRNA increased in the mammary gland, and decreased in the uterus, but the mice treated in such a manner did not develop the marked morphological changes characteristic of long-term hormone administration. (FIG. 9C). Thus, these findings confirmed that the regulation of Hunk expression by estradiol and progesterone is not solely a consequence of changes in mammary and uterine tissue architecture that occur in response to chronic hormone treatment, but rather that the changes appear to result from direct regulation by these hormones.

### Hunk Overexpression Results in Impaired Lactation.

Consonant with the hypothesis that Hunk plays a role in mammary gland development during pregnancy, it was noted that the number of pups successfully reared by MHK3 transgenic females was significantly lower than those of wild type animals. Many of the pups died within 1-2 days of birth, independent of pup genotype. In contrast, offspring of transgenic males mated to wild type females displayed survival rates comparable with those observed for offspring of wild type crosses. These observations suggested that the inability to successfully rear pups was due to a defect in the ability of MHK3 transgenic females to lactate.

To confirm the initial observations regarding reduced RNA content in MHK3 mammary glands, the yield was determined of total RNA (500 μg), isolated from number 3 and number 5 mammary glands harvested from either wild type or MHK3 transgenic females during mammary development at the time points shown in FIG. 11A. The average total RNA yield for each group is represented as the mean±s.e.m. At least three mice were analyzed from each group.

As expected, in wild type animals, this analysis revealed an approximately 20-fold increase in RNA yield from lactating when compared with nulliparous mammary glands, particularly seen at day 18.5 of pregnancy, and day 2 of lactation (t-test, P=0.047 and 0.0007, respectively). In contrast, the increase in RNA yield over this developmental interval was significantly lower in MHK3 transgenic glands, with the difference between wild type and transgenic glands becoming more pronounced towards late-pregnancy and lactation (wild type versus transgenic, t-test P=0.047 (day 18.5 of pregnancy) and P=0.0007 (day 2 of lactation). In fact, at day 2 of lactation only one third of the total amount of RNA was isolated from transgenic when compared with wild type glands. Non-expressing MHK3 transgenic females exhibited RNA yields that were indistinguishable from wild type animals, indicating that the reduction in RNA observed in MHK3 animals was dependent upon expression of the Hunk transgene (FIG. 11A).

These data suggest impaired mammary development in MHK3 animals during pregnancy and lactation. Consistent with the presence of a lactation defect in MHK3 mice, it was further noted that to mammary glands from pregnant or lactating transgenic animals contained lower amounts of RNA as compared with their wild type counterparts. The amount of total RNA isolated from wild type murine mammary glands is highly dependent upon the developmental stage, and can increase almost two orders of magnitude from the nulliparous state to the peak of lactation. The dramatic increase in RNA content during pregnancy and lactation as compared with nulliparous animals is, therefore, due to a combination of increased epithelial cell number and increased milk protein gene expression by individual alveolar epithelial cells.

### Hunk Overexpression Decreases Epithelial Proliferation During Mid-Pregnancy.

Lobuloalveolar development during pregnancy involves both proliferation and differentiation of alveolar epithelial cells. Alveolar cell proliferation occurs primarily during the first two trimesters of pregnancy, while alveolar differentiation occurs in a graded and progressive manner throughout pregnancy. To determine whether the decrease in RNA yield obtained from MHK3 transgenic glands during pregnancy is related to a decrease in cellular proliferation in these mice, BrdU incorporation rates were compared in epithelial cells from wild type and transgenic mammary glands (FIG. 11B).

Wild type and MHK3 transgenic female mice at different developmental stages were pulse labeled with BrdU before sacrifice. Number 4 mammary glands were harvested from each at day 12.5 and day 18.5 of pregnancy, and day 2 of lactation. At least 3-transgene-expressing mice and 3-wild type mice were analyzed for each time point. The relative percentage of BrdU-positive epithelial cells in the mammary glands of wild type was determined by quantitative analysis of anti-BrdU-stained sections and compared with MHK3 transgenic mice during development (FIG. 11B). Two hours after treatment injections of 50 μg BrdU/g total body weight, the cells were fixed and paraffin embedded. Then paraffin-embedded 5 μm sections were dewaxed as above, pretreated in 2N HC 1 for 20 minutes at RT, washed in 0.1 M borate buffer, pH 8.5 (×2) and rinsed in PBS. Harvested glands were fixed and stained with Carmine dye in order to visualize epithelial ducts and alveoli. BrdU immunohistochemistry was performed using the Vectastain Elite ABC Kit (Vector Laboratories), rat anti-BrdU IgG (Vector Laboratories), and a secondary biotinylated rabbit anti-rat IgG antibody, according to manufacturer instructions. Sections were counter-stained with Methyl Green as described above.

BrdU incorporation was detected using an anti-BrdU antibody followed by ABC detection method (Vector Laboratories). The percentage of BrdU-positive epithelial cells was determined after normalizing nuclear area to the average nuclear size of either BrdU positive or negative cells. The fraction of BrdU-positive and negative nuclei in the epithelial cells was quantified by color segmentation analysis of digitally captured images using Image-Pro Plus software (Media Cybernetics LP, Silver Spring, Md.). At least four different fields/animal, and 3-animals/time point were analyzed for BrdU incorporation. A significant difference in the fraction of BrdU-positive cells was observed between wild type and transgenic mammary glands only at day 12.5 of pregnancy (t-test, P=0.004).

As predicted based upon the similar morphology of wild type and transgenic mammary glands in nulliparous animals (data not shown), no significant difference in the percentage of BrdU-positive cells was observed between wild type and transgene-expressing mammary glands harvested from nulliparous animals.

Moreover, a dramatic increase in epithelial proliferation was observed at day 6.5 of pregnancy both in wild type and transgenic animals relative to nulliparous females. In contrast, at day 12.5 of pregnancy, epithelial proliferation rates remained high in wild type glands, but dropped markedly in glands from MHK3 animals (wild type versus transgenic at day 12.5, t-test, P=0.004). By comparison, no differences in epithelial proliferation rates were observed between wild type and transgenic glands at day 18.5 of pregnancy. Furthermore, no differences in apoptosis rates were observed between wild type and MHK3 transgenic glands during virgin development, pregnancy or lactation, as evidenced by similar levels of TUNEL-positive cells (data not shown). Since MMTV-Hunk transgene expression levels in MHK3 animals are roughly comparable in the mammary gland throughout pregnancy and do not coincide with the observed defect in proliferation, it was concluded that Hunk overexpression inhibits mammary epithelial proliferation specifically during mid-pregnancy.

### Example 4

### Hunk Expression and Overexpression

### Hunk Overexpression Impairs Lobuloalveolar Development.

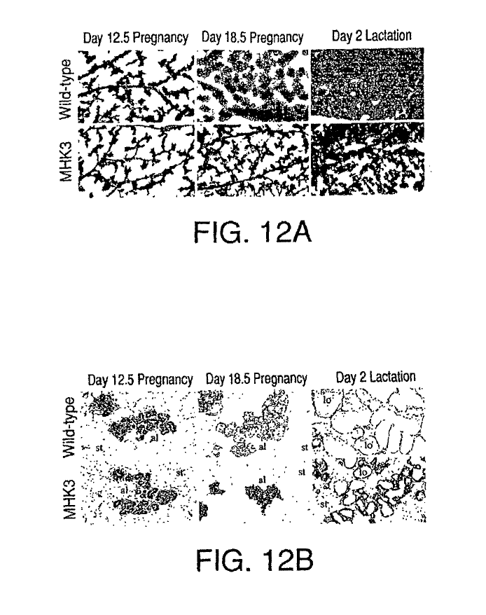

The finding above, of increased pup death among offspring of MHK3 females, when taken together with the decreased RNA content of mammary glands from lactating MHK3 animals, suggested that MHK3 female glands may have a defect in lobuloalveolar development. To address this hypothesis directly, MHK3 transgenic females were sacrificed at different stages of pregnancy and lactation for morphological analysis. However, analysis of both whole mounts and Hematoxylin and Eosin stained sections at day 6.5 and day 12.5 of pregnancy revealed no obvious morphological differences between the mammary glands of wild type and MHK3 transgenic animals, despite the fact that epithelial cell proliferation is markedly impaired in MHK3 female mice at day 12.5 of pregnancy (FIG. 11B, FIG. 12, and data not shown).

In contrast, marked morphological differences were observed between wild type and transgenic animals at day 18.5 of pregnancy. Analysis of whole mounts and Hematoxylin and Eosin stained sections at this stage of development consistently showed decreased lobuloalveolar development in MHK3 transgenic animals (FIG. 12). In addition to their larger size, alveoli in wild type mice at day 18.5 of pregnancy contained copious amounts of lipid, whereas those of MHK3 mice did not.

In addition to the abnormalities observed at day 18.5 of pregnancy, decreased lobuloalveolar development was also observed in MHK3 females at day 2 of lactation. Normally during lactation the mammary gland is filled with casein-secreting lobules, such that by whole-mount analysis the gland is entirely opaque, and by histological analysis no white adipose tissue is seen (FIG. 12). In contrast, lobuloalveolar units in lactating Hunk-overexpressing transgenic animals were smaller, and appeared less developed by whole-mount analysis as compared with wild type and non-expressing MHK3 females (FIG. 12A, and data not shown). Consequently, only half of the mammary fat pad of lactating MHK3 mice was occupied by secretory alveoli (FIG. 12B).

While this may be due in part to decreased epithelial cell proliferation observed during mid-pregnancy, morphometric analysis of Hematoxylin and Eosin stained sections from MHK3 mice at day 18.5 of pregnancy, and day 2 of lactation, revealed that compared with their wild type counterparts, the mammary glands of MHK3 animals consist of a normal number of alveoli that are uniformly smaller and less differentiated morphologically. They do not contain a smaller number of morphologically normal alveoli. (FIG. 12B, and data not shown). Moreover, alveoli in lactating transgenic animals were less distended with milk when compared with wild type glands. In contrast, similar analyses performed on the mammary glands of non-expressing MHK3 transgenic animals during lactation revealed no morphological defects (data not shown). These observations indicate that dysregulated expression of Hunk impairs terminal differentiation of the mammary gland during late pregnancy and lactation in a manner potentially distinct from the observed defect in epithelial proliferation.

### Hunk Overexpression Inhibits Mammary Epithelial Differentiation.

The dramatic changes in epithelial differentiation that occur in the mammary gland during lobuloalveolar development are reflected on a molecular level by the tightly regulated and temporally ordered expression of genes for milk proteins (Robinson et al., Development 121:2079-2090 (1995)). While steady-state mRNA levels for each of these genes typically increase throughout pregnancy, each gene undergoes a maximal increase in expression at a characteristic time during pregnancy. These differential expression profiles permit individual genes to be classified as early (β-casein), intermediate (κ-casein, lactoferrin), late-intermediate (WAP, whey acidic protein), or late (ε-casein) markers of mammary epithelial differentiation (Robinson et al., 1995; D'Cruz, unpublished). As such, the expression of these genes can be used as a molecular correlate for the extent of mammary epithelial differentiation. Accordingly, analysis of temporal expression patterns of milk protein genes permits the degree of lobuloalveolar differentiation to be reproducibly and objectively determined at the molecular level.

To confirm that the defect in lobuloalveolar development observed in MHK3 transgenic mice included a defect in differentiation, and was not simply a consequence of reduced epithelial cell numbers, the expression of a panel of molecular differentiation markers was examined in wild type and MHK3 animals during lobuloalveolar development. Probes for milk protein gene expression were: β-casein, nucleotides 181-719 (GenBank Accession number X04490); κ-casein, nucleotides 125-661 (GenBank Accession number M10114); lactoferrin, nucleotides 993-2065 (GenBank Accession number D88510); WAP, nucleotides 131-483 (GenBank Accession number X01158), and ε-casein, nucleotides 83-637 (GenBank Accession number V00740).

If the defect in lobuloalveolar development was solely due to reduced epithelial cell mass, then the absolute level of expression of milk protein genes in MHK3 animals should be similar to that observed in wild type animals when normalized for epithelial content. Similarly, if alveolar cells present in MHK3 glands differentiate normally during pregnancy, then the levels of expression of early, mid and late differentiation markers relative to each other should be similar to that observed in wild type animals. As such, the observation that the absolute levels of expression of multiple differentiation markers are reduced despite normalizing for epithelial content, or that the expression of these differentiation markers relative to each other is altered compared to wild type animals, would indicate that mammary epithelial differentiation is impaired in MHK3 animals and is independent of the observed proliferation defect.

To determine whether MHK3 animals manifest a defect in differentiation in addition to the defect in proliferation demonstrated above, mRNA expression levels were determined at day 6.5 of pregnancy (FIG. 13A), day 12.5 of pregnancy (FIG. 13B), day 18.5 of pregnancy (FIG. 13C) or at day 2 of lactation (FIG. 13D), for a panel of early (β-casein), intermediate (κ-casein, lactoferrin), late intermediate (WAP), and late (ε-casein) markers of mammary epithelial differentiation in mammary glands from transgenic and wild type animals.

Although few if any morphological differences were noted in transgenic mice before day 18.5 of pregnancy, when normalized to β-actin expression, steady-state levels of expression for all five milk protein genes were reduced in mammary glands from MHK3 transgenic mice compared with wild type mice, beginning as early as day 6.5 of pregnancy and persisting throughout pregnancy and into lactation. In contrast, expression levels of the epithelial cell marker, cytokeratin 18, did not differ significantly between wild type and transgenic glands at any stage of pregnancy or lactation when normalized to β-actin expression (FIG. 13). Although β-actin levels did not change significantly on a per cell basis during pregnancy and lactation, the enormous contribution of the expression of milk protein genes to the total RNA pool results in an apparent decrease in the expression of reference genes when comparing equal amounts of total RNA (FIGS. 7 and 13). The magnitude of this dilutional effect correlated with the differentiation state of the mammary gland. Thus, the lower levels of expression of milk protein genes observed in the less differentiated MHK3 glands results in a less severe dilutional effect and apparent increases in β-actin and cytokeratin 18 expression in the mammary glands of MHK3 animals, as compared with wild type animals at day 18 of pregnancy, and day 2 of lactation. Therefore, in aggregate, the data set forth herein indicate that the reduced expression of differentiation markers in MHK3 animals during pregnancy and lactation is not simply due to a reduction in epithelial cell content and suggests that mammary glands from Hwrøk-overexpressing transgenic mice are less differentiated than wild type glands at each stage of lobuloalveolar development.

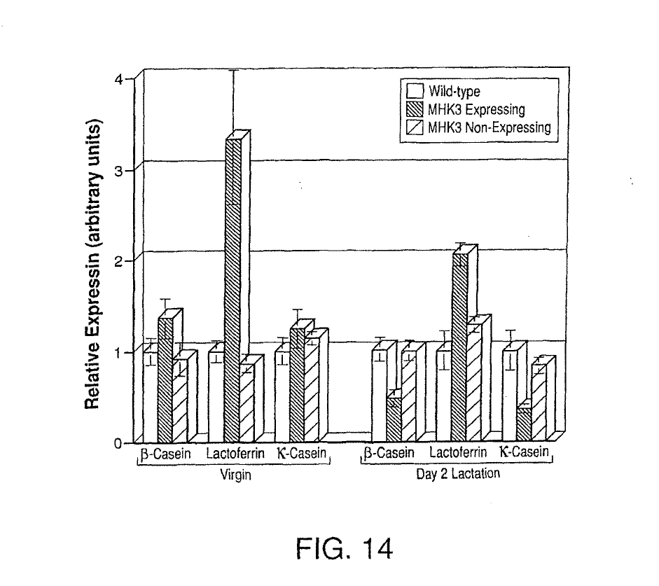

As further controls for these experiments, expression of milk protein genes was analyzed in non-expressing MHK3 transgenic females at day 2 of lactation (FIGS. 13 and 14, and data not shown). No differences in the expression either of cytokeratin 18 or of alveolar differentiation markers were observed between non-expressing MHK3 glands and glands from wild type mice, consistent with the lack of morphological or functional defects in non-expressing MHK3 glands.

FIG. 13E summarizes a multivariate regression analysis of expression products shown in FIGS. 13A-13D, demonstrating the effects of transgene expression and developmental stage on the natural logarithm of cytokeratin 18 and expression levels of milk protein genes. All expression levels were normalized to β-actin. The average effect of transgene expression (Effect) on the expression of each milk protein gene is represented as the natural logarithm of the average fold-difference between transgenic and wild type values. The respective P value (significance of transgene effect) is shown for each milk protein gene. Notably, the transgene expression had no effect on cytokeratin 18 expression, and resulted in an average decrease in the expression levels of differentiation markers ranging from 2.0-fold (β-casein) to 6.5-fold (6-casein). The R² value represents the degree to which the difference in the observed data from the null hypothesis is due to transgene expression. The P value for the significance of the regression model was <0.01 for all differentiation markers shown.

Northern analyses of expression products shown in FIGS. 13A-13D were quantified by phosphorimager quantification methods (as described in the previous Examples; Marquis et al., 1995)(see FIG. 13F). As above, the ³²P-labeled cDNA probe for Hunk encompassed nucleotides 275 to 793 (GenBank Accession number AF167987). The number of mice analyzed in each group were: 4 Wt, 5 Tg (d6.5); 3 Wt, 3 Tg (d12.5 and d18.5); and 4 Wt, 4 Tg, 4 non-expressing Tg (d2 Lact). Together, these findings strongly suggested that the abnormalities in mammary epithelial differentiation observed in MHK3 mice are due to MMTV-Hunk transgene expression, rather than to site-specific integration effects, such as the insertional disruption of an endogenous gene.

To further analyze the impact of MMTV-Hunk transgene expression on lobuloalveolar development, a multivariate regression analysis was performed on the above normalized gene expression is data to quantitate the effects of transgene expression on mammary epithelial differentiation during a developmental interval from day 6.5 of pregnancy to day 2 of lactation (FIG. 13E, 13F). This analysis revealed that the expression of four epithelial differentiation markers, β-casein, κ-casein, WAP, and ε-casein, was significantly lower in the mammary glands of transgenic animals compared with wild type animals across all developmental time points.

No differences were observed in cytokeratin 18 expression between wild type and transgenic glands, confirming that normalization to β-actin expression was sufficient to control for differences in epithelial cell content. These results indicated that the mammary glands of MHK3 animals were significantly less differentiated than wild type glands throughout pregnancy and into lactation.

Interestingly, the average reductions in mRNA expression levels observed for the late differentiation marker, ε-casein (Tg effect=−1.87; 6.5-fold), and the late-intermediate differentiation marker, WAP (Tg effect=−1.53; 4.6-fold), were considerably more pronounced than the reductions in expression observed for the early differentiation marker, β-casein (Tg effect=−0.70; 2.0-fold), and the intermediate differentiation marker, κ-casein (Tg effect=−0.94; 2.6-fold) (FIG. 13). The observation that transgene expression had a greater effect on the expression of late differentiation markers compared with early differentiation markers indicated that late events in mammary epithelial differentiation are disproportionately affected during lobuloalveolar development in MHK3 mice. This finding was consistent with the morphological defects observed in these mice during late pregnancy. Hunk upregulates lactoferrin expression in MHK3 mice.

Surprisingly, while the expression of all 5 epithelial differentiation markers examined was reduced in the mammary glands of MHK3 transgenic animals throughout pregnancy, expression of the gene for lactoferrin was actually higher in transgenic animals compared with wild type animals at day 2 of lactation (FIGS. 13D and 14). This finding led to an analysis of the impact of Hunk overexpression on lactoferrin expression in nulliparous MHK3 mice. Sample sizes were 16, 10 and 8 animals, respectively, for adolescent mice, and 4 animals per group for lactation points. Northern hybridization analysis and quantification was performed, as above, on 3 μg (virgin) or 5 μg (day 2 lactation) total RNA, isolated from mammary glands using ³²P-labeled cDNA probes specific for milk protein genes as indicated in FIG. 14.

Consistent with results obtained in lactating MHK3 animals, steady-state levels of lactoferrin mRNA were significantly higher in the mammary glands of nulliparous MHK3 expressing transgenic animals compared with either non-expressing MHK3 transgenic animals or age-matched nulliparous wild type animals, after normalization to fi-actin (FIG. 14). This effect was surprising. Therefore to determine whether the effects of Hunk overexpression on lactoferrin expression may be more specific than the generally inhibited mammary epithelial differentiation that results from Hunk overexpression during pregnancy and lactation, gene expression patterns were compared in wild type and MHK3 nulliparous transgenic glands using oligonucleotide-based cDNA microarrays. These microarray studies revealed that, of the approximately 5500 genes analyzed, the gene for lactoferrin is one of only 16 genes whose expression changes by more than 2.5-fold in transgenic animals, when compared with wild type glands. As noted above, the mammary glands of nulliparous MHK3 animals are morphologically indistinguishable from those of wild type littermates. Thus, the data indicate that the effects of Hunk overexpressionon lactoferrin gene regulation are relatively specific, and are unlikely to be secondary to marked abnormalities in mammary gland morphology or to global changes in gene expression.

In contrast to lactoferrin, mRNA expression levels of the epithelial differentiation markers, β-casein, κ-casein, α-lactalbumin (Lalba—Mouse Genome Informatics, location), WDNM1(Expi—Mouse Genome Informatics), and WAP, in adolescent nulliparous females, were not significantly affected by Hunk overexpression (FIG. 14, and data not shown). Consistent with this finding, the rate of ductal elongation and extent of epithelial side-branching in mammary glands from 5- to 6-week-old nulliparous transgenic mice was comparable with that observed in wild type mice, as analyzed by whole-mount and histological analysis (data not shown). These observations indicated that Hunk does not cause precocious differentiation of the mammary gland during puberty, but may specifically activate pathways resulting in lactoferrin upregulation. Similarly, the observation that lactoferrin expression is up-regulated in the mammary glands of lactating MHK3 animals, despite the global inhibitory effect of Hunk overexpression on mammary epithelial differentiation during late pregnancy and lactation, confirmed the conclusion that the effects of Hunk on lactoferrin expression are distinct from those on mammary epithelial differentiation.

Finally, as shown, the treatment of mice with 17β-estradiol and progesterone results in the rapid and synergistic up-regulation of Hunk expression in the mammary gland, indicating that the up-regulation of Hunk expression in response to hormones is not a consequence of the marked changes in epithelial differentiation or epithelial cell number that occur either during early pregnancy or in response to the chronic administration of 17β-estradiol and progesterone. Interestingly, unlike the effect of steroid hormones on Hunk expression in the mammary gland, treatment of mice with 17β-estradiol either alone or in combination with progesterone results in down-regulation of Hunk expression in the uterus. The opposing effects of combined estradiol and progesterone treatment on Hunk expression in the mammary gland and uterus is reminiscent of the dichotomous effects of these hormones on epithelial proliferation in these tissues. As such, the data shows that the effect of steroid hormones on Hunk expression in the mammary gland and uterus parallels the dichotomous response of these tissues to estradiol and progesterone, thus providing additional support for the role of Hunk as a downstream effector of estrogen and progesterone, and offers an explanation for the dichotomous response of these tissues to steroid hormones.

### Example 5

### HUNK Expression in Human Primary Ovarian and Colon Tumors

To investigate the potential involvement of HUNK, or a cell type in which HUNK is expressed, in human breast, ovarian and colon carcinogenesis, HUNK expression levels were determined in a panel of human primary breast, ovarian and colon cancers along with benign tissue samples from each of these organs. An RNase protection analysis was performed, as above, using 30 μg of total RNA isolated from tumors hybridized with a ³²P-labeled antisense riboprobe specific for HUNK or for β-actin. As a negative control, tRNA was used for comparison.

RNA was isolated from 6 benign breast tissue samples and from 46 primary breast tumors obtained after surgery. An RNase protection analysis was performed using 10 μg of total RNA hybridized with a ³²P-labeled antisense riboprobe specific for HUNK, cytokeratin 18 (CKl 8) or for β-actin. HUNK and β-actin expression levels were quantified by phosphorimager analysis, and HUNK expression levels were normalized to either CK 18 or β-actin for each sample. HUNK expression levels in breast tumors were compared with benign tissue. Normalized HUNK expression levels in the benign tissues was set equal to 1.0. This analysis demonstrated that among all breast tumors, HUNK is expressed at a level that is 2.2-fold lower than in benign ovarian tissue. Moreover, when analyzed by subsets, 76% of all breast tumors were found to exhibit HUNK expression levels that were 5.0-fold lower than the average HUNK expression levels observed in benign tissue. Further analysis of HUNK expression as a function of breast tumor grade revealed that HUNK expression correlates negatively with tumor grade with poorly-differentiated (p=0.036) and moderately-differentiated (p=0.0029) tumors exhibiting lower levels of HUNK expression than benign tissues. Finally, expression of HUNK was also found to be decreased in both ductal carcinomas and lobular carcinomas.

In a similar manner, RNA was isolated from 16 benign ovarian tissue samples and from 22 primary ovarian tumors obtained after surgery. An RNase protection analysis was performed using 10 μg of total RNA hybridized with a ³²P-labeled antisense riboprobe specific for HUNK or for β-actin. HUNK and β-actin expression levels were quantified by phosphorimager analysis, and HUNK expression levels were normalized to β-actin for each sample. HUNK expression levels in ovarian tumors were compared with benign tissue. Normalized HUNK expression levels in the benign tissues was set equal to 1.0. This analysis demonstrated that HUNK is expressed in ovarian tumors at a level that is 10.3-fold higher than in benign ovarian tissue (p=0.0000034). Further analysis of HUNK expression as a function of ovarian tumor grade revealed that HUNK expression correlates positively with tumor grade with poorly-differentiated tumors and moderately-differentiated tumors exhibiting higher levels of HUNK expression than well-differentiated tumors.

Finally, RNA was isolated from 17 benign colon tissue samples and from 24 paired primary colon tumors obtained after surgery (e.g., benign samples were taken from the same patient as the tumor samples). An RNase protection analysis was performed using 10 μg of total RNA hybridized with a ³²P-labeled antisense riboprobe specific for HUNK or for β-actin. HUNK and β-actin expression levels were quantified by phosphorimager analysis, and HUNK expression levels were normalized to β-actin for each sample. HUNK expression levels in colon tumors were compared with benign tissue. Normalized HUNK expression levels in the benign tissues was set equal to 1.0. This analysis demonstrated that HUNK is expressed in colon tumors at a level that is 1.9-fold higher than in benign colon tissue (p=0.035). Notably, this elevated level of expression of HUNK in colon tumors was primarily due to the massive overexpression of HUNK in a subset of colon tumors. Specifically, 4 tumors exhibited expression levels that were greater than 10 standard deviations from the mean of benign tissues. Finally, expression of the HUNK kinase has been shown to be increased in a subset of human colon carcinomas compared to benign tissue, and to be positively associated with tumor grade.

### Example 6

### Role of HUNK in Mammary Tumor Metastasis

### Animal and Tissue Preparation

Mice were housed under barrier conditions with a 12-hour light/dark cycle. For histological analysis, tumors were fixed in 4% paraformaldehyde O/N and transferred to 70% ethanol prior to paraffin embedding; sections were cut and stained with Hematoxylin and Eosin.

Cloning of Hunk cDNA

Nucleotides 276 to 793 of Hunk (GenBank Accession #AF 167987) was used to screen a human fetal brain cDNA library (Stratagene, La Jolla, Calif.) as previously described (Gardner et al., Genomics 63:46-59 (2000)). Two overlapping clones spanning the entire ORF were sequenced on both strands to obtain the composite nucleotide and amino acid sequence.

### Analysis of HUNK Expression

RNA was prepared as previously described (Marquis et al., Nat. Genet. 11:17-26 (1995); Rajan et al., Proc. Natl. Acad. Sci. USA 93:13078-83 (1996)). Nucleotides 359 to 582 of human HUNK or nucleotides 1142 to 1241 of β-actin (GenBank accession #X03672) were used as probes for RNAse protection analysis (Marquis et al., Nat. Genet. 11:17-26 (1995)).

### Quantitative Real-Time RT-PCR

cDNA was generated from total RNA using the Superscript™ First-strand synthesis system as per manufacturer's protocol (Invitrogen, Carlsbad, Calif.). Sequences of primers and probes are as follows: TBP primers (Forward: 5′ GGA GCT GTG ATG TGA AGT TTC CTA TAA 3′ (SEQ ID NO:19); Backward: 5′ AAC CAG GAA ATA ACT CTG GCT CAT AA 3′ (SEQ ID NO:20)), HUNK primers (Forward: 5′CAC CAA AGC CCT CCT GAA GGA 3′ (SEQ ID NO:21); Backward: 5′ GCC ACA CAA TTG GAA TCT GAG GTT T 3′ (SEQ ID NO:22)), TBP probe (5′ VIC-AGG CCT TGT GCT CAC CCA CCA ACA-TAMRA 3′ (SEQ ID NO:23)), HUNK probe (5′ FAM-CTC CAA GTC CAG CTT CCC CGA CAA AG-TAMRA 3′ (SEQ ID NO:24)).

### Generation and Analysis of Hunk-Deficient Mice

A 129/Sv mouse genomic library (Stratagene, La Jolla, Calif.) was screened with a Hunk cDNA fragment (nt 1-706) (Gardner et al., Genomics 63:46-59 (2000)). The Hunk gene was disrupted by replacement of a 1.1 kb fragment containing the putative promoter and exon 1 of Hunk with a pGKneo cassette flanked by LoxP sites. Twenty-five micrograms of linearized vector was inserted into 1×10⁷ E14 ES cells by electroporation and subsequently selected in 300 μg/ml G418. Properly target clones, as well as resulting mice, were screened by Southern hybridization using a 3′ Xho I/Xmn I flanking probe. Lung protein lysates (7 mg) were subjected to immunoprecipitation and immunoblotting with Hunk-specific C-terminal antisera as previously described (Gardner et al., Development 127:4493-509 (2000)). Tumor and Metastasis Analysis

Hunk-deficient animals were crossed to mice harbouring an MMTV-c-myc transgene (Leder et al., Cell 45:485-95 (1986)). Hunk heterozygous, MMTV-c-myc mice were backcrossed to Hunk heterozygous animals. MMTV-c-myc female animals of each Hunk genotype were mated twice, then monitored twice weekly for mammary tumors. Mice possessing tumors with a maximum diameter of 20 mm were sacrificed and organs were examined for metastases using a Leica Wild MZ8 dissection microscope.

For transplant experiments, Hunk wild-type and heterozygous metastatic tumors, and Hunk-deficient non-metastatic tumors were digested at 37° C. for 3 hours in collagenase, followed by a 15 min digestion in trypsin. Single cell suspensions were injected into athymic nude mice (tail vein 5×10⁵ cells, mammary fat pad 5×10⁶ cells). Mice were sacrificed either eight weeks post-injection (tail vein assays) or when tumors achieved a maximum diameter of 20 mm (fat pad assays). Soft agar growth assays were performed in 6-well dishes as previously described using 4×10⁶ cells/well (Lo et al., Cancer Res. 64:6127-36 (2004)). Colonies were counted after two weeks.

### Oligonucleotide Microarray Analyses

Approximately 5 μg of total RNA was used for each tumor sample. Biotinylated cRNA was generated and hybridized to Affymetrix HGU95A (human tumors) or MGU74Av2 (murine tumors) oligonucleotide arrays. Sample preparation, raw data collection and gene expression analysis were performed as described previously (Master et al., Mol. Endocrinol. 16:1185-203 (2002); Master et al., Genome. Biol. 6:R20 (2005)).

### Generation of Murine and Human HUNK-Expression Signatures

The list of differentially expressed genes comparing Hunk-expressing and non-expressing primary human breast cancers was comprised of the union of the list of genes exhibiting differential expression in at least 24 of 30 pairwise comparisons as assessed by MAS5 (Affymetrix, Santa Clara, Calif.) and the list of differentially expressed genes identified by ChipStat with p<0.0035. This combined analytical approach has been demonstrated to identify differentially expressed genes with a high degree of sensitivity and specificity (Master et al., Genome. Biol. 6:R20 (2005)). All gene lists were further filtered to include only those probe sets identified as present in at least 50% of the samples in any sample group.

For the mouse expression signature, the list of differentially expressed genes was comprised of the union of three gene lists. These were 1) the list of genes demonstrating differential expression in at least 32 of 36 pairwise comparisons as assessed by MAS5; 2) the list of differentially expressed genes identified by ChipStat with p<0.00028; and 3) the intersection of the list of genes differentially expressed in at least 28 of 36 pairwise comparisons (MAS5) and identified by ChipStat at p<0.00077.

### Comparison to External Gene Lists

For list overlap analysis, the significance of the size of the overlap between two gene lists was assessed using the hypergeometric distribution. For comparison of lists between mouse and human data sets, murine genes were first mapped to homologous human gene using HomoloGene (NCBI).

Clustering of published human breast cancer data sets was preformed using Ward's hierarchical clustering method with the mouse or human Hunk-expression signatures. For the data set of van't Veer et is al. (van 't Veer et al., Nature 415:530-6 (2002)), genes were included in the analysis only if p<0.01 in more than 5 samples. In the data sets of Sorlie et al. (Sorlie et al., Proc. Natl. Acad. Sci. USA 100:8418-23 (2003)) and Ma et al. (Ma et al., Cancer Cell 5:607-16 (2004)) only genes available on at least 95% of the arrays and whose variances were in the top 25% were included. Z-scores of the log-ratios for each gene were used in the analysis. Kaplan-Meier survival curves and log rank analysis were generated using Prism 4.0 (GraphPad Software, San Diego, Calif.).

The proportions of genes having predictive value were determined by per-gene ANOVA analysis and Tukey-Kramer multiple comparison tests. Only genes having FDR-adjusted ANOVA p-value less than 0.05, and whose expression was significantly different between at least one pair of tumor clusters with significant hazard ratio were considered to be predictive.

Multivariate Cox's proportional hazard model was used to calculate the hazard ratios among the tumor clusters and to assess their significance. A proportional hazard model was also used to assess the prognostic power of tumor clustering results after adjusting for common prognostic indicators. When analyzing variables with N levels (N>2), N-1 dummy variables were used. The correlations between the clustering results and commonly used prognostic indicators were assessed by Fisher's exact test, and tumor size was analyzed as a binned variable. Identification and designation of tumor subtypes was performed as previously described (Sorlie et al., Proc. Natl. Acad. Sci. USA 100:8418-23 (2003)).

### Human HUNK is Differentially Expressed in Human Breast Cancers.

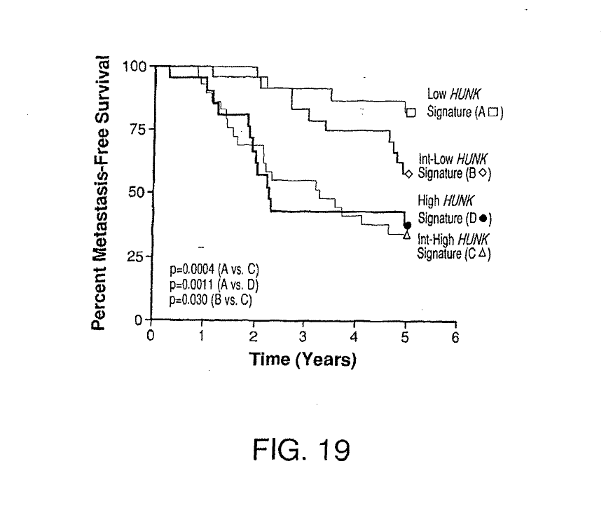

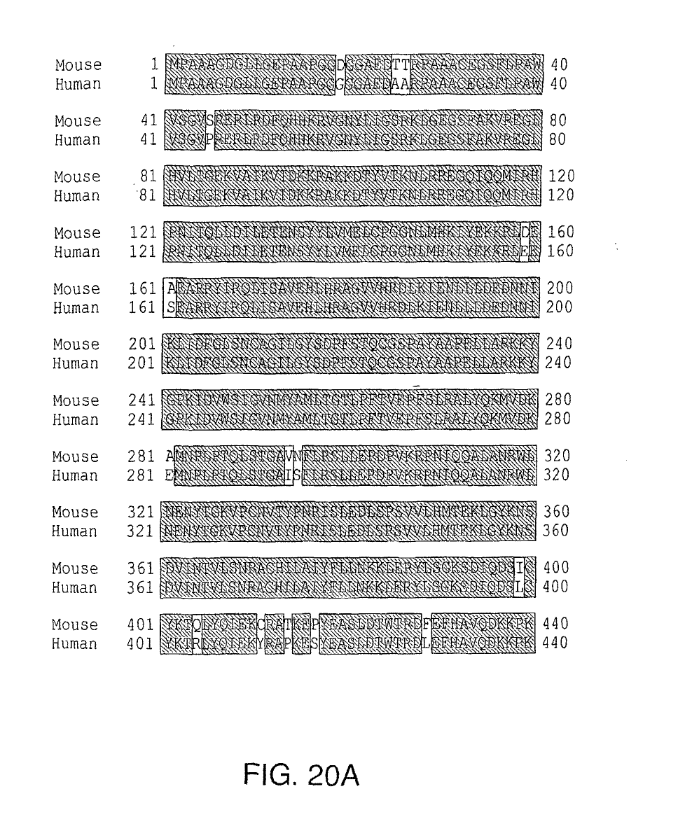

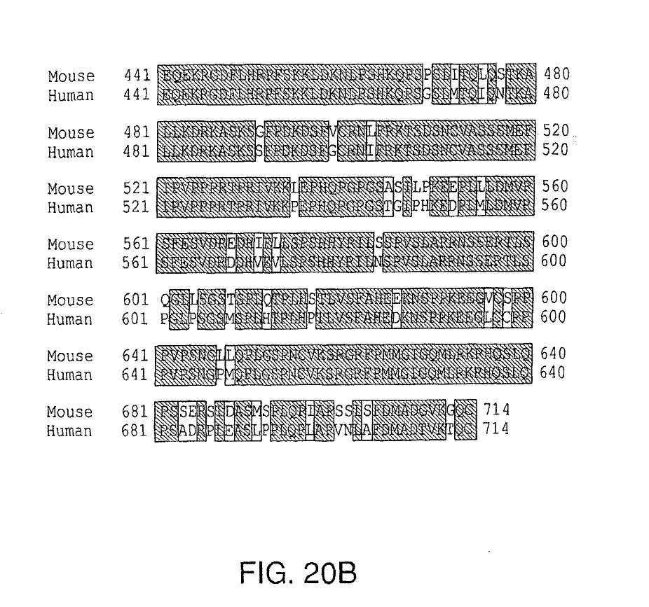

Murine Hunk was previously cloned and defined its expression during mammary gland development (Gardner et al., Genomics, 63:46-59 (2000); Gardner, et al., Development, 127:4493-4509 (2000)). To extend these analyses, multiple cDNA clones for HUNK were isolated from a human foetal brain cDNA library. Sequence analysis yielded a composite cDNA spanning an open reading frame of 714 amino acids (GenBank accession #NM_{—}014586). Review of human genome data indicated that a single HUNK isoform exists that is 92% identical to murine Hunk at the amino acid level (FIG. 19).

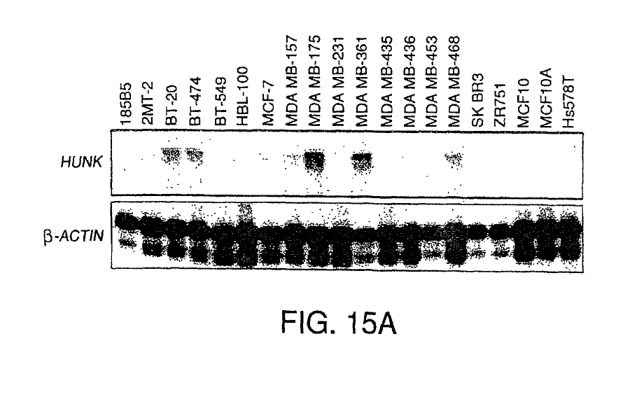

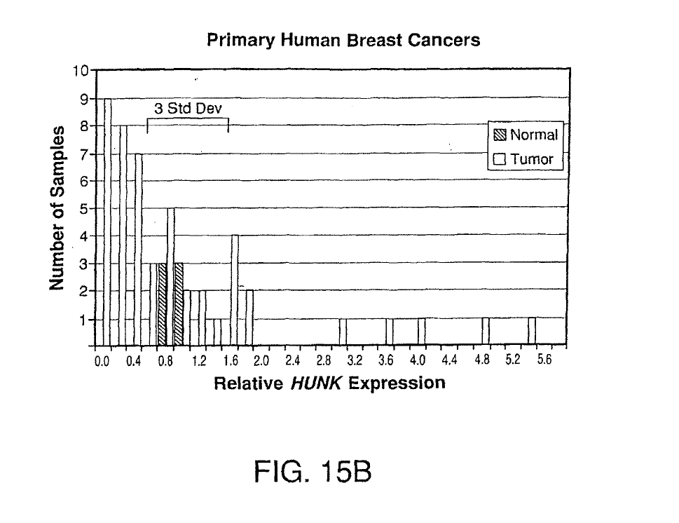

Given the defects in mammary epithelial proliferation and differentiation that was previously identified in MMTV-Hunk transgenic mice (Gardner et al., Development 127:4493-509 (2000)), it was investigated whether HUNK plays a role in human breast cancer. As an initial step towards examining this hypothesis, RNase protection and real-time quantitative RT-PCR analyses were used to demonstrate that HUNK exhibits a ˜200-fold range of expression (FIG. 15A and data not shown). Interestingly, HUNK expression did not correlate with transformation, HER2/neu amplification or oestrogen receptor (ER) status. Consistent with the non-uniform expression of HUNK in cell lines, expression in human primary breast cancers exhibited a greater than 70-fold range of expression (FIG. 15B). While HUNK-expression levels in normal breast tissue was relatively constant, 27% of human breast cancers (13/48) expressed HUNK at levels three standard deviations or more greater than the mean of normal breast tissue. Conversely, fifty percent of tumors (24/48) expressed HUNK at levels less than three standard deviations of the mean. This highly heterogeneous pattern of expression suggested that HUNK may be differentially expressed among different breast cancer subtypes.

### HUNK Expression Predicts Metastasis-Free Survival in Breast Cancer Patients.

To identify molecular features characteristic of tumors expressing high levels of HUNK, gene expression profiles of eleven primary human breast cancers were analyzed using Affymetrix HGU95A oligonucleotide arrays. Six tumors expressed HUNK at levels at least four-fold lower than normal breast tissue (HUNK-low), whereas five tumors expressed levels of HUNK at least three-fold higher than normal breast tissue (HUNK-high). Of ˜9000 genes analyzed, 204 were consistently upregulated and 306 were consistently downregulated in HUNK-high tumors (HUNK-expression signature) using established algorithms (Master et al., Mol. Endocrinol. 16:1185-203 (2002); Master et al., Genome. Biol. 6:R20 (2005)).

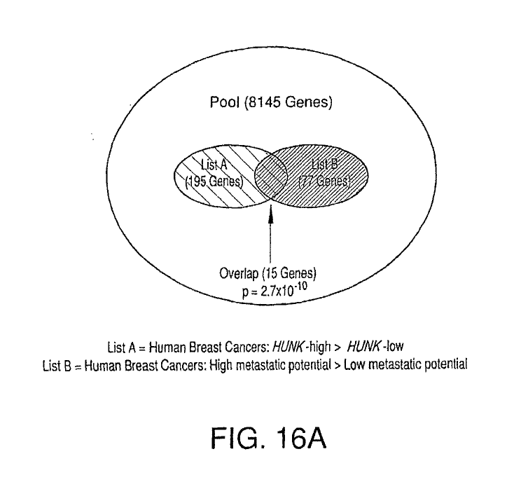

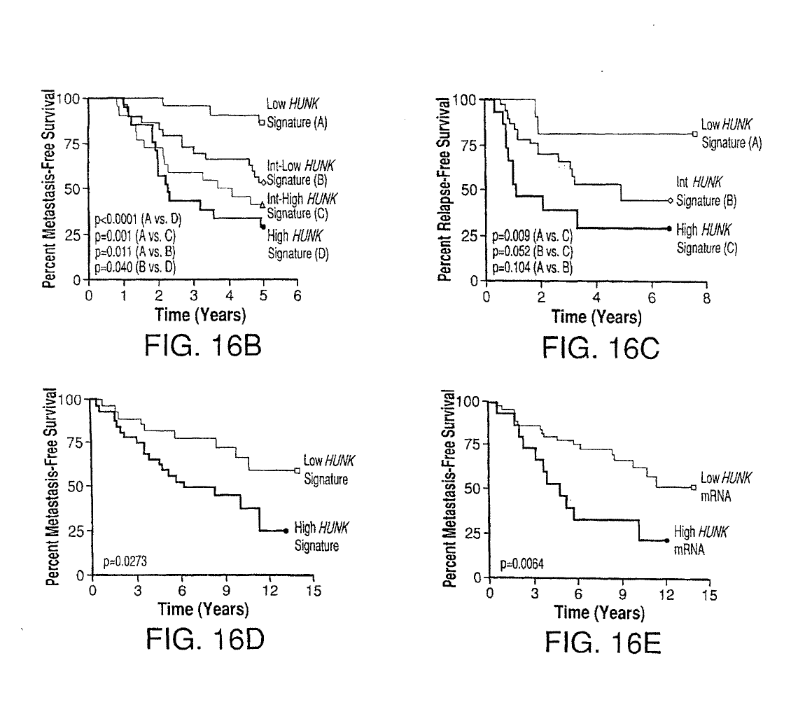

Based on these findings, if molecular differences exist between Hunk-expressing and non-expressing tumors that are relevant to the behavior of human breast cancers, the genes that are differentially expressed between these two groups might participate in cellular processes that contribute to the clinical outcome of breast cancer patients. Therefore, the list of genes that correlate with high HUNK expression in human breast cancers was compared to the gene list described by van't Veer et al. (van 't Veer et al., Nature 415:530-6 (2002)) that identifies human breast cancers with high metastatic potential (FIG. 16A). This latter list was generated by microarray analysis of 97 lymph node-negative breast cancers in women who were free of metastases at presentation and who were monitored for a period of five years following the surgical removal of their tumor. Since these gene lists were derived using different microarray platforms, the gene “pool” used in this comparison was restricted to genes represented on both arrays. Moreover, since inclusion of genes not expressed in either experiment would lead to an overestimation of the significance of any overlap between these lists, genes that were detected in less than half of the examined primary tumor samples were excluded. A total of 8145 genes met both criteria. Of these, 195 correlated with high HUNK expression (List A) and 77 correlated with high metastatic potential (List B). Given the list sizes and gene population size, only two genes would be expected to appear on both lists by chance. In contrast, 15 genes were common to both lists, demonstrating a highly significant degree of similarity between the HUNK signature and genes associated with high metastatic potential (p=2.72×10⁻¹⁰, hypergeometric test). This result suggests that the molecular features that define HUNK expressing human breast cancers are related to those that identify human breast cancers with high metastatic potential.

The genes comprising the HUNK-expression signature were then used to hierarchically cluster an independent set of human breast cancers with known clinical outcome. If HUNK expression is indeed related to metastatic outcome, it was likely that tumors would segregate—in an unsupervised manner—into clusters that differed with respect to metastatic potential. As shown in FIG. 16B, hierarchical clustering using the HUNK signature segregated early-stage, node-negative breast cancers (van 't Veer et al., Nature 415:530-6 (2002)) into four groups. Consistent with the finding linking the gene expression profiles of HUNK-expressing tumors to molecular features of highly metastatic human breast cancers, patients with tumors exhibiting the highest degree of similarity to the HUNK-expression signature (Cluster D) had the shortest metastasis-free survival (FIG. 16B, Cluster A vs. Cluster D, Hazard Ratio (HR)=9.45, p<0.0001). The elevated risk of metastasis associated with the HUNK-expression signature in this cohort was higher than that associated with commonly used indicators of clinical outcome including HER2/NEU amplification (HR=4.10), ER status (HR=2.32) and tumor grade (HR=3.30) (Table 3).

TABLE 3
Hazard ratios of prognostic indicators.

| | van't Veer et al. | | Sorlie et al. | | Ma et al. | |
|---|---|---|---|---|---|---|
| | HR | p-value | HR | p-value | HR | p-value |
| HUNK signature | 9.45 | 0.0004 | 6.58 | 0.015 | 2.39 | 0.033 |
| HUNK expression | N/A | N/A | N/A | N/A | 2.72 | 0.006 |
| Lymph node status | N/A | N/A | 1.33 | 0.414 | 0.97 | 0.947 |
| Tumor size | 1.73 | 0.004 | 1.11 | 0.810 | 1.33 | 0.269 |
| HER2 status | 4.10 | 0.002 | 3.04 | 0.042 | 1.90 | 0.385 |
| ER status | 2.32 | 0.005 | 2.59 | 0.021 | N/A | N/A |
| Tumor grade | 3.30 | 0.001 | 5.08 | 0.031 | 2.01 | 0.073 |

Clinical outcome hazard ratios (HR) and associated p-values for the HUNK-expression signature, HUNK expression and clinically utilized prognostic variables are indicated for data sets of van't Veer et al. (van't Veer et al., Nature 415:530-6 (2002)), Sorlie et al. (Sorlie et al., Proc. Natl. Acad. Sci USA 100:8418-23 (2003)) and Ma et al. (Ma et al., Cancer Cell 5:607-16 (2004)).

Additionally, tumors with an intermediate degree of similarity to the HUNK-signature exhibited intermediate risks of metastatic relapse (Cluster A vs. Cluster C, HR=6.21, p=0.001; Cluster A vs. Cluster B, HR=4.37, p=0.011). Interestingly, the prognostic significance of the HUNK signature was not limited to the 15 previously identified metastasis-associated genes (van 't Veer et al., Nature 415:530-6 (2002)) since over 71% (258) of the genes in the HUNK-associated gene signature have predictive value within this data set. Consistent with this, elimination of the 15 genes identified in FIG. 16A did not abrogate the ability of the HUNK-signature to predict clinical outcome.

To extend these findings, a similar analysis was performed with the data sets of Sorlie et al. (Sorlie et al., Proc. Natl. Acad. Sci. USA 100:8418-23 (2003)) and Ma et al. (Ma et al., Cancer Cell 5:607-16 (2004)), consisting of locally advanced breast cancers and ER-positive breast cancers, respectively. The HUNK-expression signature segregated locally advanced breast cancers into three clusters with significantly different potential for relapse (FIG. 16C, Cluster A vs. Cluster C, HR=6.58, p=0.009). Similarly, ER-positive breast cancers were segregated by the HUNK signature into two clusters with significantly different potential for metastatic relapse (FIG. 16D, HR=2.39, p=0.027). As with early stage, node-negative breast cancers, HUNK was a stronger predictor of metastasis- and relapse-free survival than other commonly utilized prognostic indicators, including tumor size, tumor grade, lymph node status, ER-status and HER2/Neu amplification (Table 3). Thus, the HUNK-associated gene expression signature identified herein represents a robust predictor of metastasis-free survival in women with breast cancer.

As a further indication of the relationship between HUNK expression and metastatic outcome, it was investigated whether HUNK mRNA expression was able to predict metastasis-free survival. A single probe directed against HUNK in the experiments conducted by Ma et al. (Ma et al., Cancer Cell 5:607-16 (2004)), exhibited expression significantly higher than background as well as significant variance across samples, consistent with the range of expression previously observed in primary breast cancers. Use of this probe as a marker of HUNK expression confirmed that—similar to the HUNK signature—HUNK mRNA expression was significantly associated with metastatic outcome (FIG. 16E). Specifically, women with tumors expressing high levels of HUNK (upper quartile) had a significantly worse metastatic prognosis than women with tumors expressing low levels of HUNK (lower three quartiles, HR=2.72, p=0.0064). Consistent with the hypothesis that the HUNK signature, in fact, reflects HUNK gene expression, significant overlap was evident between tumor clusters segregated based on HUNK expression and those segregated based on the HUNK-expression signature (p=0.018, Fisher's exact test), Thus, HUNK expression itself, as well as the HUNK-expression signature, identifies human breast cancers with high metastatic potential.

### Hunk is Required for Metastasis of Mammary Tumors in Mice.

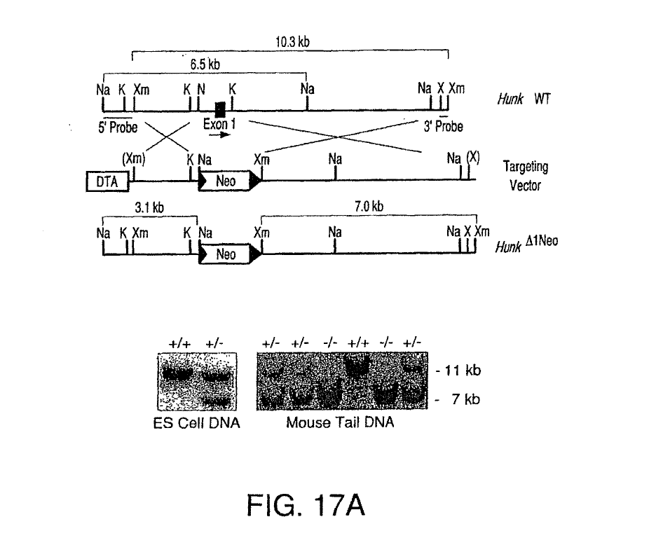

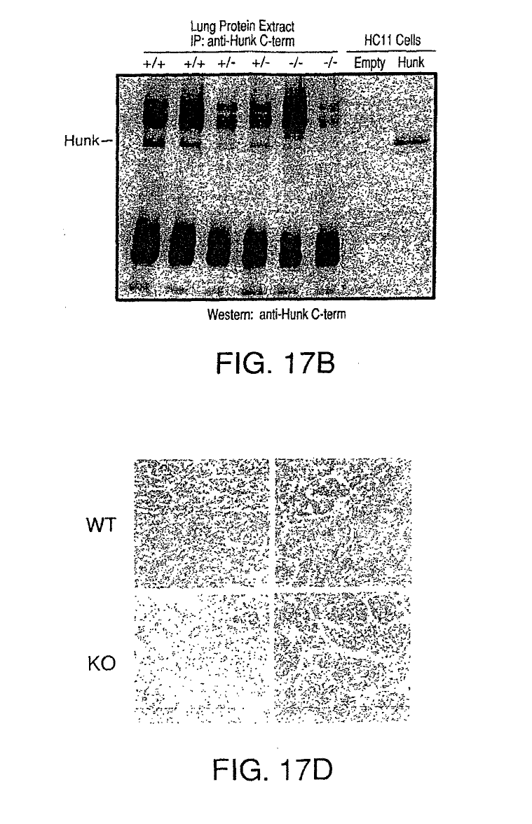

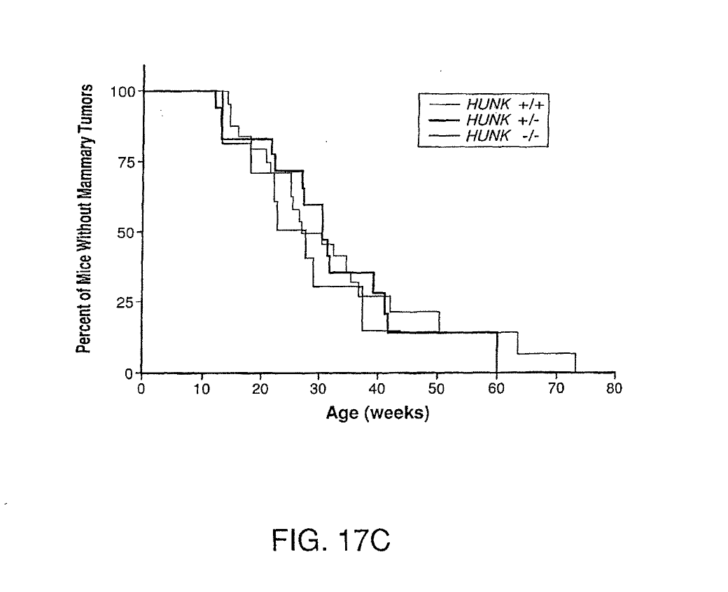

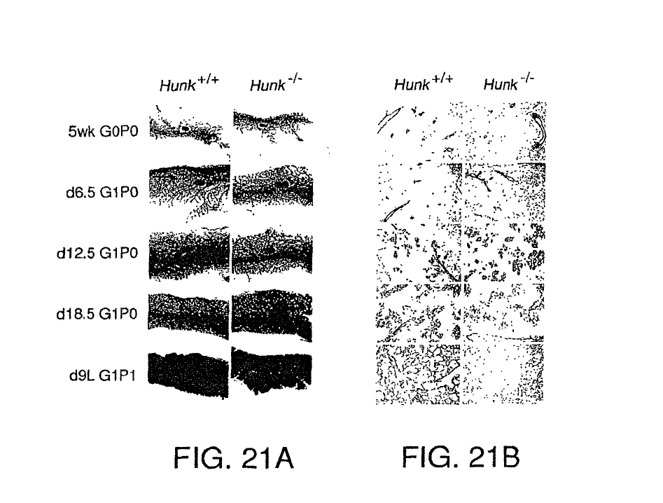

The relationship between HUNK expression and the likelihood of metastasis could result if HUNK merely served as a marker for highly aggressive breast cancers. Alternatively, HUNK could directly influence the metastatic behavior of breast cancers. To distinguish between these possibilities, a Hunk-deficient mouse strain was generated and characterized. Standard gene targeting techniques were used to delete the putative promoter and the first exon of the Hunk gene (FIG. 17A). Hunk-deficient mice did not express detectable Hunk protein, yet exhibited similar viability, longevity, fertility, and organogenesis compared to littermate controls (FIG. 17B and data not shown). Likewise, an extensive analysis of mammary gland development failed to reveal morphological, histological, or functional differences among Hunk wild type, heterozygous or homozygous mutant mice (FIGS. 21 A-B). These findings demonstrated that Hunk is not required for murine development, including that of the mammary gland.

To assess the role of Hunk in mammary tumorigenesis and metastasis, Hunk-deficient mice were crossed to mice bearing an MMTV-c-myc transgene. Mice overexpressing c-myc develop mammary tumors that metastasize to the lung (Leder et al., Cell 45:485-95 (1986)). Moreover, c-MYC is overexpressed in 40-50% of human breast cancers and is associated with aggressive tumor behavior and decreased relapse-free survival (Naidu et al., Int. J. Mol. Med. 9:189-96 (2002); Guerin et al., Oncogene Res. 3:21-31 (1988); Deming et al., Br. J. Cancer 83:1688-95 (2000)). MMTV-c-myc transgenic mice that were wild-type, heterozygous, or deficient for Hunk, were monitored twice weekly for mammary tumor development. No differences in tumor latency, multiplicity, or growth rates were observed among the different Hunk genotypes (FIG. 17D and data not shown). Furthermore, tumors from wild type and Hunk-deficient mice were histologically indistinguishable (FIG. 17D). Thus, Hunk is not required for c-myc-induced mammary tumorigenesis.

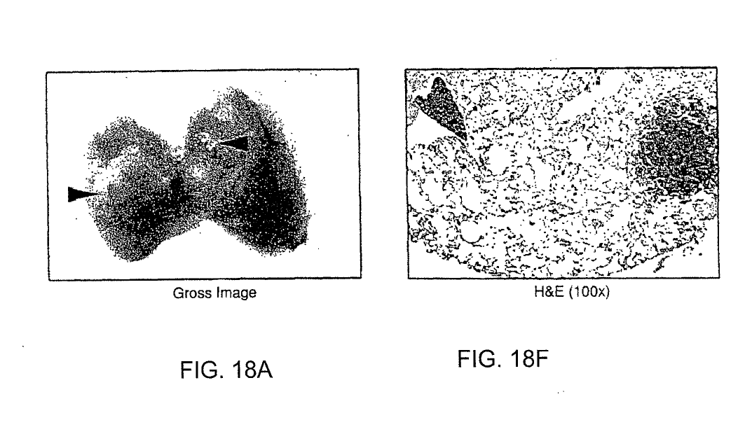

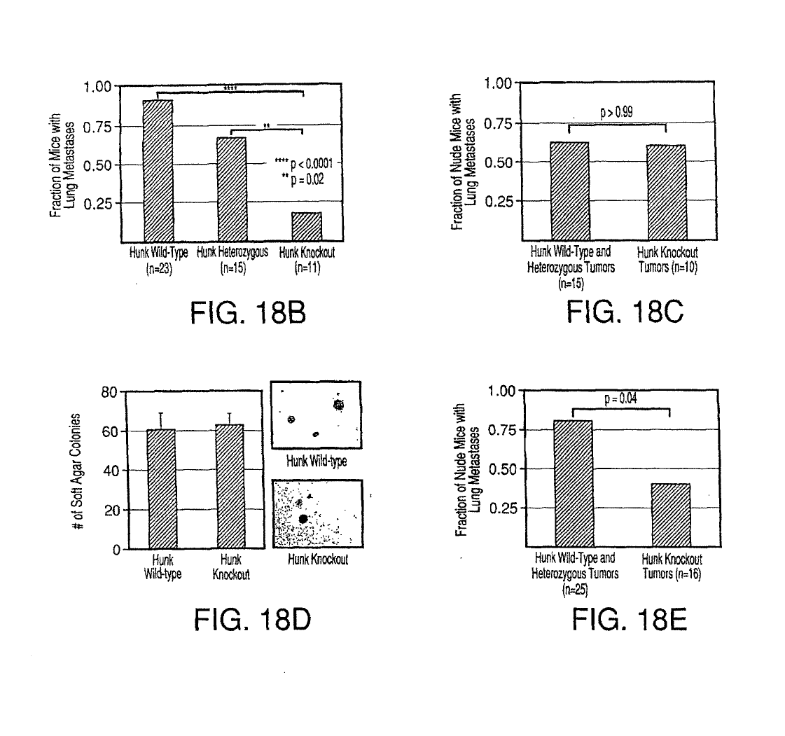

To test the hypothesis that Hunk contributes to mammary tumor metastasis, mice possessing similarly-sized tumors were sacrificed and examined at necropsy for distant metastases. A subset of animals had grossly detectable lung lesions that were determined by histological evaluation to be metastatic epithelial tumors (FIG. 18A). Notably, the frequency of mammary tumor metastasis in Hunk-deficient mice was ˜5-fold lower than that observed in Hunk wild type mice (FIG. 18b, p0.0001). Hunk-heterozygous mice exhibited an intermediate metastatic rate that was significantly higher than that observed in homozygous mutant mice. These results demonstrate that Hunk is required for the efficient metastasis of c-myc-induced murine mammary tumors.

To characterize the nature of the defect in metastatic potential conferred by Hunk mutation, primary mammary tumor cells from Hunk wild type, Hunk heterozygous and Hunk-deficient mice were introduced via tail vein injection directly into the circulation of nude mice. These tumor cells displayed a similar metastatic frequency across Hunk genotypes (FIG. 18C). Consistent with this, Hunk wild type and Hunk-deficient tumor cells exhibited similar anchorage-independent growth characteristics (FIG. 18D). In contrast, primary tumor cells orthotopically transplanted into the mammary fat pads of nude mice recapitulated the difference in metastatic potential observed between Hunk wild type and Hunk-deficient tumors (FIG. 18E). Taken together, these results suggest that the metastatic defect observed in Hunk-deficient tumor cells is attributable to a decreased ability to enter the circulation rather than an impaired ability to survive within the circulation or at distant sites.

### Murine Tumors Recapitulate Features of Human Breast Cancer.

The finding that tumors arising in Hunk-deficient mice display reduced metastatic potential, as described elsewhere herein, was consistent with the initial hypothesis that HUNK plays a critical role in the metastasis of human breast cancers. However, since the biology of human tumors and mouse tumors is clearly not identical (Van Dyke et al., Cell 108:135-44 (2002)), assessment was required to determine just how closely the Hunk-expression signature derived from c-myc-induced mammary tumors in Hunk-deficient mice resembled the Hunk-expression signature derived from human breast cancers. To accomplish this, the gene expression profiles of six c-myc-induced tumors from Hunk-deficient animals were compared with those of six c-myc-induced tumors from Hunk wild-type animals. Unsupervised hierarchical clustering segregated tumors into two distinct groups corresponding to Hunk genotype (FIG. 19). This demonstrates that while Hunk wild type and Hunk-deficient tumors appear histologically similar, they are biologically and molecularly distinct.

Genes expressed at higher levels in Hunk wild type compared to Hunk-deficient tumors were compared to genes expressed at higher levels in Hunk-expressing human breast cancers. This analysis demonstrated a significant overlap between the murine and the human expression signatures (p=5.2×10⁻⁶, hypergeometric test). Moreover, the murine and human expression signatures were found to cluster Hunk-expressing and non-expressing human breast cancers in a significantly similar manner (p=0.015, Fisher's exact test). Thus, the molecular features distinguishing c-myc-induced murine mammary tumors in Hunk wild type compared to Hunk-deficient mice reflect multiple molecular features of human breast cancers selected solely on the basis of their expression of HUNK.

The significant similarity observed between murine and human Hunk-expression signatures, to coupled with the ability of the Hunk-expression signature to predict clinical outcome, suggested that this Hunk-deficient mouse model recapitulates biological features relevant to the metastasis of human breast cancers. To explore this possibility, the murine Hunk-expression signature was used to hierarchically cluster the data set of van't Veer et al. (van 't Veer et al., Nature 415:530-6 (2002)) (FIG. 19). Human breast cancers analyzed in this manner segregated into four groups that exhibited significant differences in metastatic potential (FIG. 19). As with the human HUNK signature, the groups of tumors with the highest metastatic potential were associated with the high Hunk-expression signature (Cluster A vs. Cluster D, HR=6.1, p=0.0011). A remarkably high degree of similarity was observed between the tumor clusters defined by the mouse Hunk signature and those defined by the human HUNK signature (p=1.10×10⁻⁴¹, Fisher's exact test). Additionally, similar to results obtained with the human Hunk-expression signature, over 65% (263) of genes in the murine Hunk-expression signature were found to have predictive value for clinical outcome. These results demonstrate that the murine Hunk-expression signature is a strong predictor of metastasis-free survival in women with breast cancer, and further suggest that the Hunk-deficient mouse model accurately reflects key aspects of human breast cancer biology.

### HUNK is a Robust and Independent Predictor of Metastatic Outcome.

As demonstrated elsewhere herein, a gene expression signature associated with HUNK expression can predict clinical outcome in a broad spectrum of breast cancer patients. Also as set forth elsewhere herein, genetic evidence in the mouse demonstrates that Hunk is required for efficient breast cancer metastasis. It was further shown herein that HUNK expression is associated with a greater risk of metastatic relapse than currently used clinical prognostic indicators (Table 3).

In theory, the prognostic power of the HUNK signature could be attributable to its association with a commonly used prognostic indicator of breast cancer outcome. To test this hypothesis, contingency table, along with Fisher's exact analysis, was used to assess the association between breast cancer clusters segregated based upon Hunk-expression signature and HER2 expression, lymph node status and tumor size. No significant association was observed between the HUNK signature and any of these parameters. In contrast, the Hunk-expression signature was strongly correlated with ER-negative cancers in the data set of van't Veer et al. (p=2.7×10⁴¹), and displayed a similar, though non-significant, skewing in the data set of Sorlie et al. (p=0.097). These results are consistent with the aggressive behavior of both ER-negative breast cancers and cancers with high HUNK expression. Nevertheless, the HUNK-expression signature was predictive in the ER-positive data set of Ma et al., as well as in the subsets of ER-positive cancers in the data sets of van't Veer et al. and Sorlie et al. (HR=3.2, p=0.003 and HR=I 3.7 p=0.006, respectively). Moreover, the Hunk-expression signature retains the ability to predict clinical outcome in both data sets even after adjusting for ER-expression using a two variable proportional hazard ratio model (Table 4).

TABLE 4
Proportional hazard ratio models.

| | van't Veer et al. | | Sorlie et al. | | Ma et al. | |
|---|---|---|---|---|---|---|
| | HR | p-value | HR | p-value | HR | p-value |
| HUNK signature | 5.15 | 0.013 | 5.34 | 0.035 | N/A | N/A |
| ER status | 1.39 | 0.561 | 1.98 | 0.108 | N/A | N/A |
| HUNK signature | 6.10 | 0.008 | 4.08 | 0.115 | 2.06 | 0.103 |
| Tumor grade | 2.10 | 0.102 | 2.42 | 0.299 | 1.48 | 0.351 |

Hazard ratios and associated p-values are listed for the HUNK-expression signature and ER-status or tumor grade using two-variable proportional hazard ratio modelling. Hazard ratios and p-values are displayed for HUNK signature and ER (top) and HUNK signature and tumor grade (bottom) after adjusting for the predictive power of the corresponding variable for data sets of van't Veer et al. (van't Veer et al., Nature 415:530-6 (2002)), Sortie et al. (Sortie et al., Proc Natl. Acad. Sci. USA 100:8418-23 (2003)) and Ma et al. (Ma et al., Cancer Cell 5:607-16 (2004)).

In contrast, the ER-status of cancers was not predictive after adjusting for HUNK. Thus, the Hunk-expression signature has prognostic power that is independent of its relationship with ER-status, whereas ER status does not have prognostic power independent of its relationship with the HUNK signature.

Similar analyses demonstrated a strong correlation between the Hunk-expression signature and histological grade in all three data sets evaluated (van't Veer et al. p=1.4×10⁻⁷, Sorlie et al. p=2.2×10⁻⁵, Ma et al. p=2.0×10⁻⁶). Proportional hazard ratio modelling demonstrated that the Hunk-expression signature remains predictive after adjusting for tumor grade with cancers in the data set of van't Veer et al. (Table 4). Thus, in early-stage lymph node-negative tumors, the Hunk-expression signature predicts metastatic outcome independent of tumor grade. Within the data sets of Sorlie et al. and Ma et al., neither the Hunk-expression signature nor tumor grade were able to predict clinical outcome independently of each other. However, clinical outcome was correlated more strongly with the HUNK-expression signature than tumor grade in each data set (p=0.115 vs. 0.299 Sorlie et al.; p=0.103 vs. 0.351 Ma et al.). These results suggest that while the HUNK signature is correlated with tumor grade in these data sets, it predicts clinical outcome more accurately than tumor grade. These findings suggest that Hunk may play a role in regulating the cellular processes that underlie the determination of tumor grade.

Finally, Sorlie et al. have used gene expression profiling to define breast cancer subtypes that display distinct propensities to metastasize and recur (Sorlie et al., Proc. Natl. Acad. Sci. USA 98:10869-74 (2001)). These subtypes, in order of increasing likelihood of recurrence, are defined as luminal A, normal-like, luminal B+C, ERBB2-positive and basal subtypes. Contingency table and Fisher's exact analysis of early stage breast cancers (van de Vijver et al., N. Engl. J. Med. 347:1999-2009 (2002)) to revealed an overrepresentation of the basal subtype within the high-HUNK expressing cluster (Cluster D, FIG. 16C and data not shown). However, the predictive power of the Hunk-expression signature was not simply due to its association with basal subtype tumors, as evidenced by differences in clinical outcome among the remaining three clusters of tumors identified using the HUNK signature. In aggregate, these clusters contained a single basal cancer and were composed primarily of luminal B subtypes is (Cluster C, FIG. 16C), luminal A subtypes (Cluster B, FIG. 16C) and luminal A and normal-like subtypes (Cluster A, FIG. 16C). Similarly, in locally advanced breast cancers (Sorlie et al., Proc. Natl. Acad. Sci. USA 100:8418-23 (2003)) the high-HUNK expressing cluster of tumors exhibited an overrepresentation of basal and luminal B cancer subtypes (Cluster C, FIG. 16D), whereas the intermediate-Hunk-expressing cluster contained a overrepresentation of ERBB2-positive and luminal A subtypes (Cluster B, FIG. 16D). Thus, the HUNK signature is broadly associated with the aggressive behavior of breast cancer subtypes, but it is not exclusively associated with a particular subtype.

### Example 7

### HUNK Kinase Activity is Essential for Metastasis

As described elsewhere herein, HUNK kinase activity is involved in metastasis of mammary tumor cells. As set forth more fully below, HUNK kinase activity is required for metastasis of breast cancer cells.

Specifically, it is disclosed for the first time herein that a cell line derived from a MMTV-myc breast cancer arising in a Hunk knockout mouse does not efficiently metastasize to the lungs of a mouse, when the cells are allowed to form a tumor in a mammary fat pad of a recipient mouse. Further experiments with the same cell line demonstrated that expression of wild type Hunk in this cell line fully restores the metastatic potential of this cell line. Expression of a mutant form of Hunk in this same cell line, wherein the mutant form of Hunk lacks kinase activity, has no effect on metastatic potential. Taken together, these results indicate that Hunk kinase activity is responsible for the metastatic phenotype of breast cancer cells.

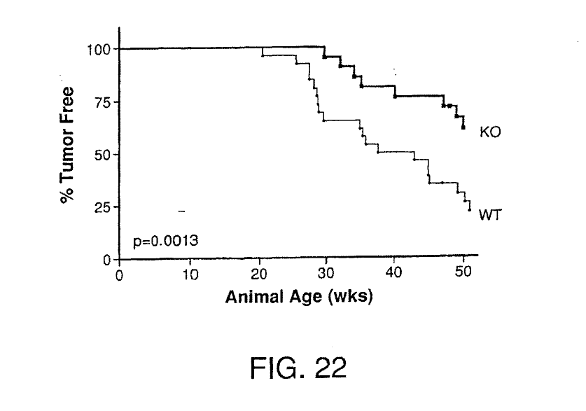

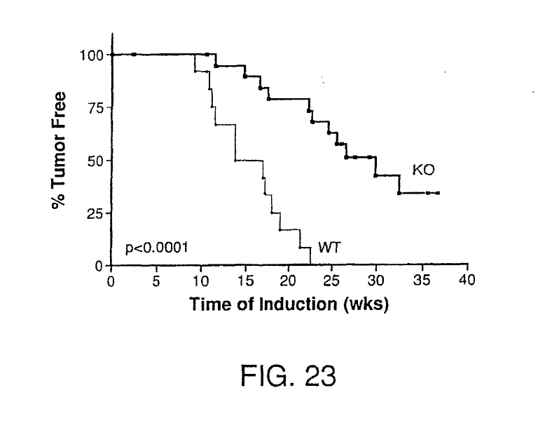

As described in detail elsewhere herein, Hunk is required for metastasis of Myc-initiated mammary tumors. Prior to the present invention, it had not been determined whether Hunk might also play a role in mammary tumor formation. It is demonstrated herein for the first time that Hunk-deficient (Hunk^{Δ1Neo/Δ1Neo}) animals were bred to the MMTV-Neu mammary tumor model. Hunk-wild type and Hunk-deficient transgenic cohorts were monitored weekly for mammary tumor development. This analysis revealed that Hunk-deficient MMTV-Neu animals display a ˜2-fold (25 week) increase in mean tumor latency when compared to Hunk wild type MMTV-Neu controls (FIG. 22). Additionally, Hunk-deficient animals displayed decreased tumor multiplicity when compared to wild type controls (FIG. 23). These results demonstrate that Hunk is required for Neu-induced tumor formation as Neu-induced tumors in Hunk-deficient animals display increased latency and decreased tumor multiplicity.

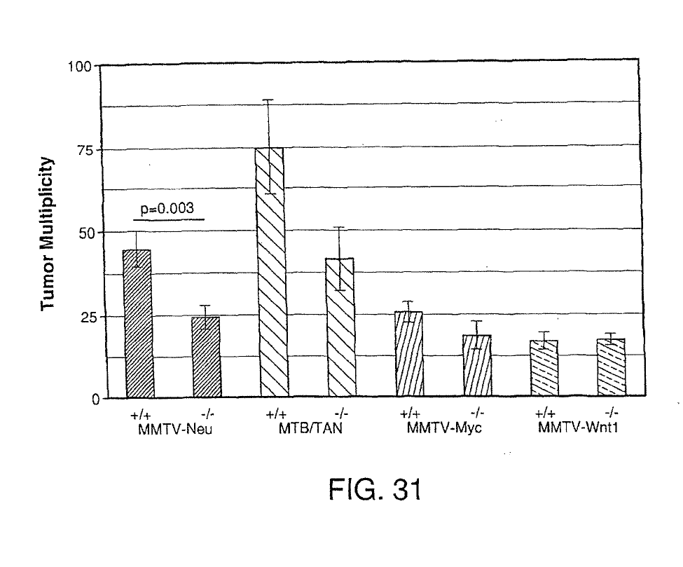

To confirm results observed in the Hunk-deficient MMTV-Neu animals, Hunk-knockout (Hunk^{Δ1/Δ1} Neo excised) animals were bred to a mammary specific doxycycline inducible Neu-transgenic is model. Animals were bitransgenic animals were induced with 0.1 mg/ml doxycycline and monitored weekly for tumor incidence. Similar to results obtained utilizing the MMTV-Neu model system, Hunk-knockout MMTV-rtTA/TetOp-NeuNT (MTB/TAN) mice displayed a ˜2-fold increase in tumor latency (FIG. 31). Additionally, while differences in tumor multiplicity are not statistically significant, the trend is comparable to that observed in the MMTV-Neu cohort (FIG. 23). Therefore, in two independent models of Neu-induced tumorigenesis, it was observed that in the absence of Hunk, tumor latency is increased and tumor multiplicity is decreased.

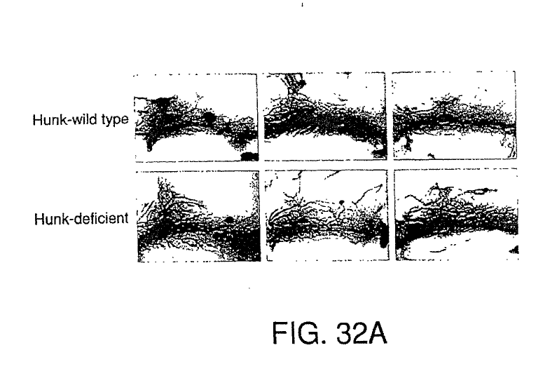

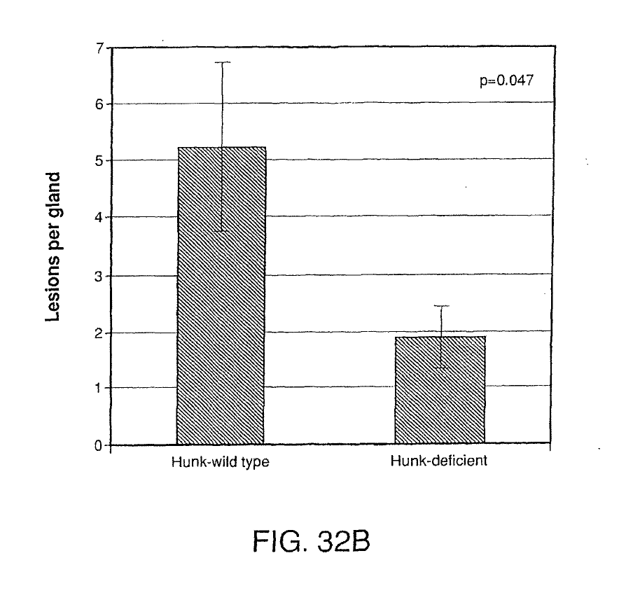

When animals were examined at necropsy it was observed that a number of the glands while not bearing bona fide tumors did bear hyperplastic lesions (FIG. 32A). To determine if the incidence of hyperplastic lesions was decreased in Hunk-deficient animals, carmine-stained non-tumor bearing number 4 mammary glands were examined. Consistent with the decreased tumor multiplicity observed in Hunk-deficient animals the incidence of hyperplastic lesions was also decreased in Hunk-deficient non-tumor bearing mammary glands (FIG. 32B). These results suggest that Hunk may regulate events early development of a Neu-induced tumor.

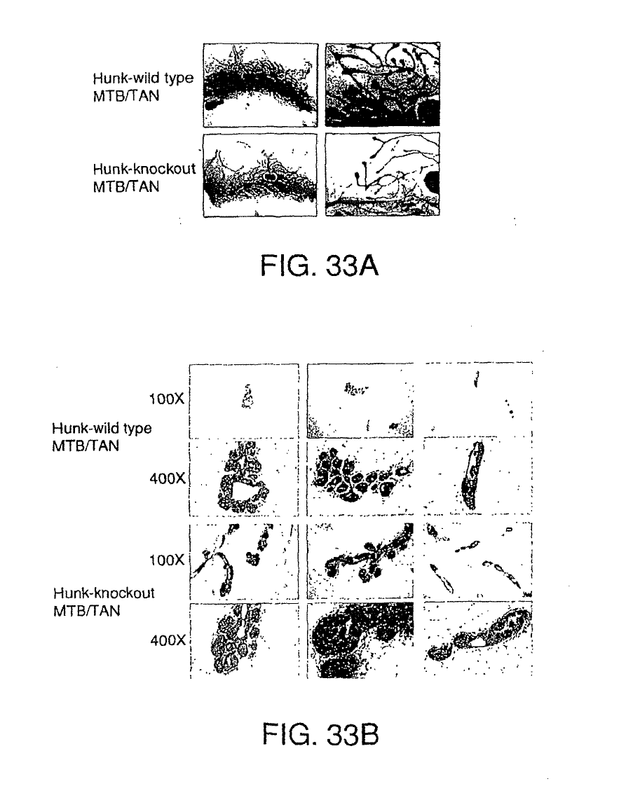

One of the advantages of utilizing an inducible oncogene system is that the oncogene can be induced for defined periods of time allowing one to examine the effects of short-term induction. In light of observations suggesting that Hunk may be required for early events in Neu-induced tumorigenesis, the morphology of carmine-stained mammary glands induced for four days with doxycycline was examined. Consistent with previously reported results, MTB/TAN mammary glands induced with doxycycline for four days displayed significant hyperplasia when compared to uninduced controls. However, no differences were observed when comparing Hunk-wild type and Hunk-knockout MTB/TAN mammary glands (FIG. 33A). Similarly, no differences were observed in hematoxylin and eosin stained sections (FIG. 33B).

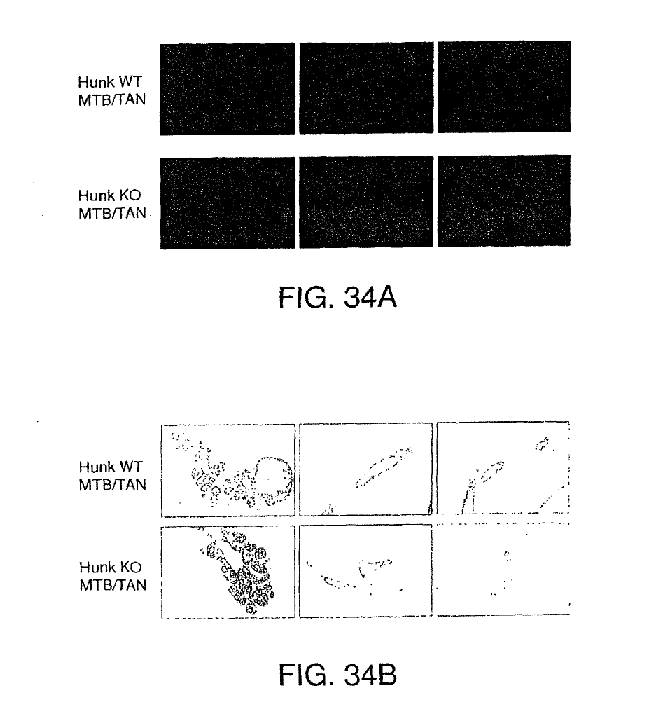

While no overt histological differences existed between Hunk-wild type and Hunk-knockout MTB/TAN mammary glands, differences in cellular proliferation and apoptosis may exist. Utilizing BrdU incorporation as a surrogate for cellular proliferation, anti-BrdU immunohistochemistry was preformed on 6 Hunk-wild type and 6 Hunk-knockout MTB/TAN mammary glands induced for 96 hrs. No statistically significant differences in epithelial cell proliferation were observed between Hunk genotypes (FIGS. 34A and 34B). Similarly, TUNEL staining was preformed on 6 Hunk-wild type and 6 Hunk-knockout MTB/TAN mammary glands induced for 96 hrs. No TUNEL positive epithelial cells were observed in either Hunk-wild type or Hunk-knockout mammary glands. Thus, the tumor latency and multiplicity defects observed in Hunk-knockout mammary glands do not effect cellular proliferation or apoptosis upon 96 hrs induction with doxycycline.

To assess the gene expression differences in Hunk-wild type and Hunk-knockout mammary glands upon 4 days of Neu induction samples were run on Affymetrix MOE34a gene arrays. Gene expression analysis identified 175 probesets which were significantly upregulated and 197 probesets which were significantly downregulated in Hunk-wild type MTB/TAN mammary glands when compared to Hunk-knockout controls. To identify the subset of genes specifically involved in Neu-induced tumorigenesis, a second list of Neu-induced and repressed genes was obtained. To determine if a significant overlap existed between those genes differentially expressed in Hunk-knockout MTB/TAN mammary glands and those genes differentially expressed in response to Neu the two lists were then overlapped. From a total population of 10,828 probesets, 175 probesets are increased in Hunk-wild type MTB/TAN mammary glands and 442 probesets are increased in response to Neu-induction. A significant number of genes (52 probesets) were found to be in common (p=4.56×10⁻³¹) between these lists of genes. Conversely, 197 probesets are repressed in Hunk-wild type MTB/TAN mammary glands and 437 probesets are repressed in response to Neu-induction. From these lists of genes significant number (78 probesets) display a significant overlap (p=3.31×10⁻⁵⁶). Strikingly there are only two probesets that show the demonstrating the inverse relationship. Thus, it appears that a significant number of genes which are normally induced by expression of Neu in the mammary gland fail to be induced in the Hunk-knockout mammary gland. Similarly, a significant number of genes which are normally repressed by expression of Neu fail to be repressed in the Hunk-knockout mammary gland.

The Pea3 family of transcription factors has been shown to be overexpressed in human breast cancers. Additionally, their activity has been shown to be required for Neu-induced murine mammary tumorigenesis. Among the three Pea3 family members, Er81 expression increases in response to Neu-induction for 4 days. In Hunk-knockout mammary glands Er81 fails to be induced upon Neu-induction. In contrast, the other two family members Pea3 and Erm are not differentially expressed between Hunk-wild type and knockout mammary glands (FIG. 6a). Interestingly, Er81 expression is restored in Hunk-knockout Neu-induced tumors, suggesting that Er81 may be critical for the development of Neu tumors.

### Example 8

### Hunk is Required for Mammary Tumor Formation Induced by the Neu Oncogene

Activation of the Neu oncogene in Hunk-knockout mice results in delayed mammary tumor development, as well as a decreased number of tumors, as compared to the Neu-mediated activation of Hunk in Hunk wild type mice.

The Neu oncogene was activated in Hunk knockout mice by breeding mice to either MMTV-Neu transgenic mice or to MMTVO-rtTA;TetO-neu transgenic mice.

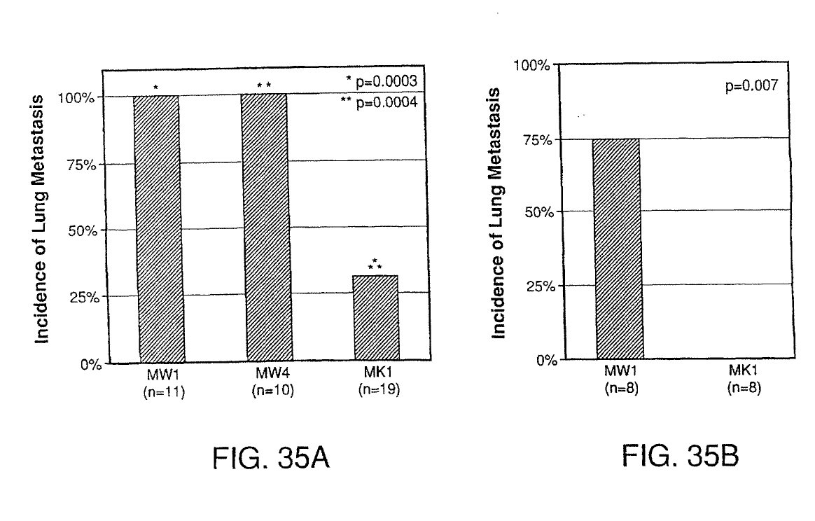

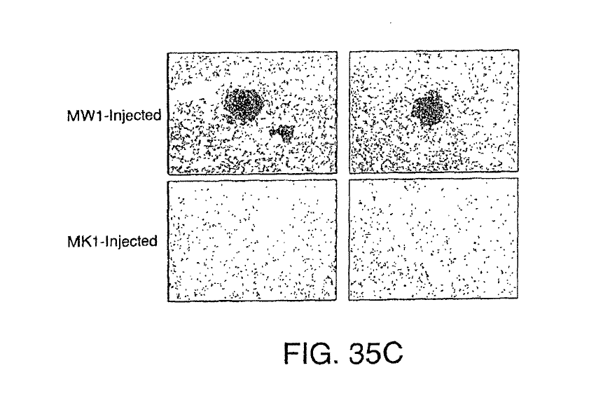

To further define the role of Hunk in mammary tumor metastasis, cell lines were established from Hunk wild type MMTV-c-myc tumors (MW 1 and MW4) and Hunk-deficient MMTV-c-myc tumors (MK1). These cell lines display indistinguishable morphology and growth characteristics when grown in vitro. As described in detail elsewhere herein, Hunk-deficient MMTV-c-myc primary tumors transplanted into the mammary fat pads of nude mice fail to metastasize when compared to wild-type controls. Upon establishment of cell lines, retention of this phenotype was assessed within the cell lines set forth herein. Cohorts of nude mice were orthotopically injected with 5×10⁶ cells, tumor growth was monitored and animals were sacrificed upon reaching a mean tumor cross-sectional area of 400 mm². No differences in tumor growth were observed, however, animals harboring MK1-derived tumors demonstrated a 3 to 6-fold decreased incidence of metastasis when compared to animals harboring MW1- or MW4-derived tumors (FIG. 35A). Additionally, when animals were sacrificed upon reaching a mean tumor cross-sectional area of 225 mm², 75% (6/8) animals harboring MW1-derived tumors metastasized whereas none of the animals harboring MK1-derived tumors exhibit metastases (0/8) (FIG. 35B). Inspection of lungs from animals harboring MK1-derived tumors by H&E did not yield any evidence of metastasis (FIG. 35C). Therefore, similar to orthotopic transplantation of Hunk-deficient tumors, MK1-derived tumors display a cell autonomous defect in metastasis.

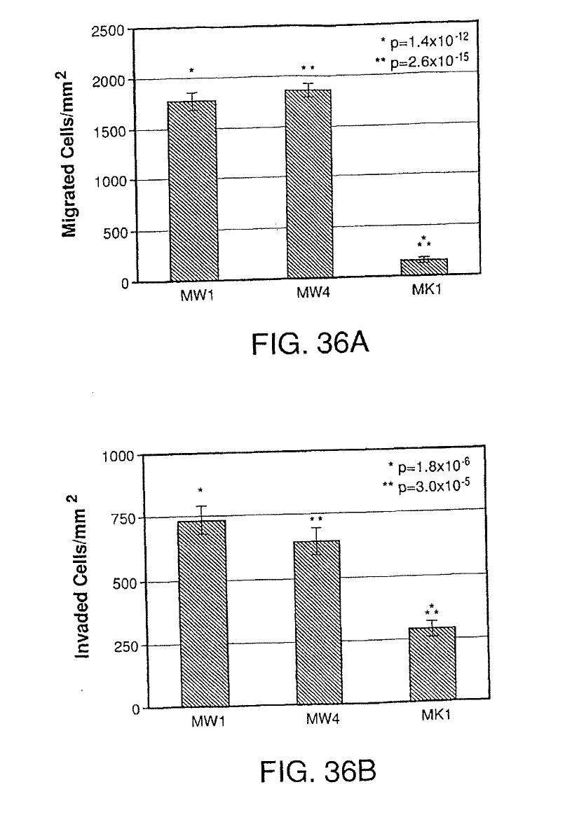

Also as described elsewhere herein, Hunk-deficient tumor cells exhibit a block in the metastatic process prior to intravasation. To further characterize this defect, it was investigated whether this defect was attributable to decreased migratory and invasive properties. Cells that translocated to the bottom of Transwell™ chamber inserts after 18 hrs were stained and counted. In multiple experiments, ˜12-fold-fewer MK1 cells migrated across the Transwell chamber membrane when compared to MW1 and MW4 (FIG. 36A). Cells were seeded in Matrigel-coated Transwell chambers to assess the invasive properties of these cell lines. Similar to results obtained with uncoated chambers, Hw<<A:—deficient cells were found to translocate less frequently (˜2.4-fold) than their wild-type controls (FIG. 36B). These results suggest that the defect in mammary tumor metastasis observed in Hunk-deficient MMTV-c-myc mice may be attributable to decreased migratory and invasive properties of Hunk-deficient cells.

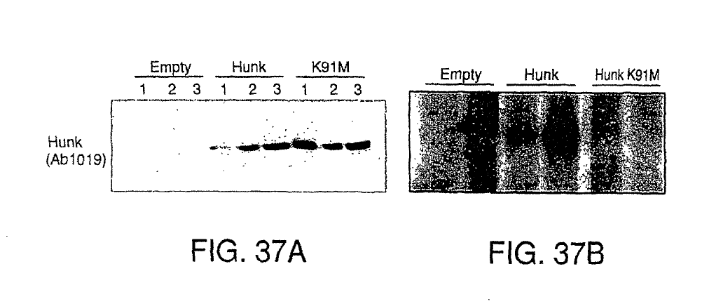

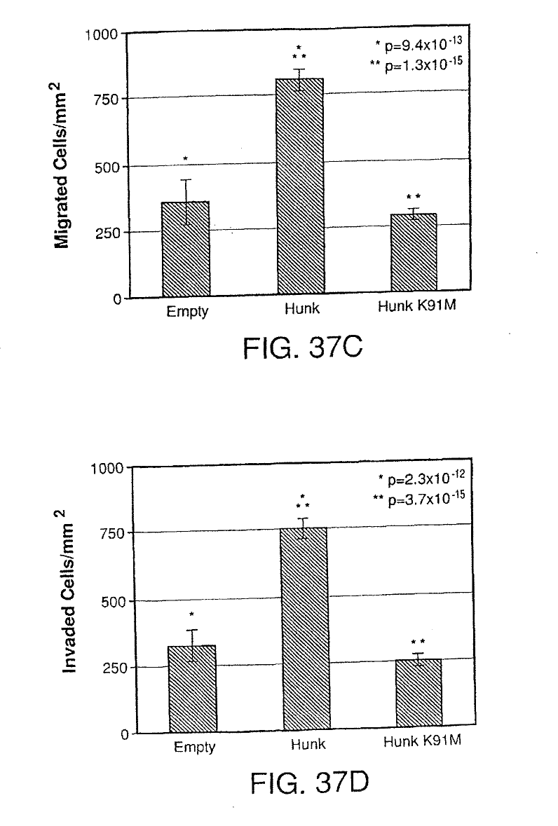

These observations are consistent with Hunk being required for mammary tumor metastasis. In a further analysis of the role of Hunk in metastasis, it was investigated whether Hunk directly regulates these cellular processes or whether Hunk is required for the establishment of a cell type predisposed to increased migration, invasion and metastasis. To assess the ability of Hunk to promote cellular migration, invasion and tumor metastasis, MK1 cells were retrovirally transduced with retrovirus expressing either wild type Hunk or a mutated, kinase-dead form of Hunk (Hunk K91M). Hunk K91 M bears a lysine to methionine substitution at a conserved residue in subdomain II, which is critical for the ATP-binding pocket. Similar substitutions have been utilized to inactivate other kinases without altering substrate binding. Independent, stably transduced pools of MK1 cells expressed readily detectable levels of both Hunk (MK1H) and Hunk K91M (MK1K). when compared to empty vector controls (MK1E) (FIG. 37A). In vitro kinase assays, demonstrate that expression of wild type Hunk is results in increased Hunk kinase activity, however, Hunk K91M transduction was not accompanied by an increase in immunoprecipitated kinase activity (FIG. 37B). These results demonstrate that the K91M substitution results in an inactive Hunk kinase.

The ability of Hunk to promote cellular migration was assessed by seeding Hunk transduced stable pools, as described elsewhere herein, in Biocoat™ control inserts. Hunk expressing MK1 cells consistently translocated ˜2.3 fold more frequently than empty vector controls and ˜2.8 fold more frequently than Hunk K91M expressing pools (FIG. 37C). Similarly, when plated on Matrigel-coated Biocoat™ inserts, Hunk expressing stable pools translocated ˜2.3 fold more frequently than empty vector controls and ˜3.0 fold more frequently than Hunk K91M expressing pools (FIG. 37D). These results demonstrate that Hunk is sufficient to increase the migratory and invasive properties of mammary tumor cells, and that the ability of Hunk to promote migration and invasion is dependent on Hunk kinase activity.

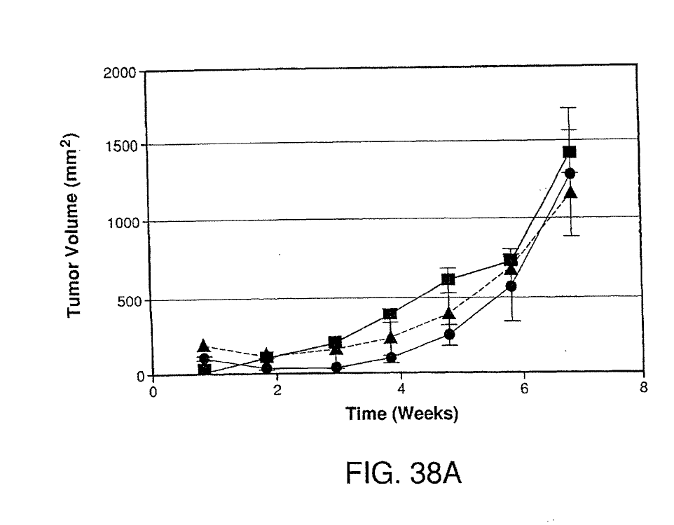

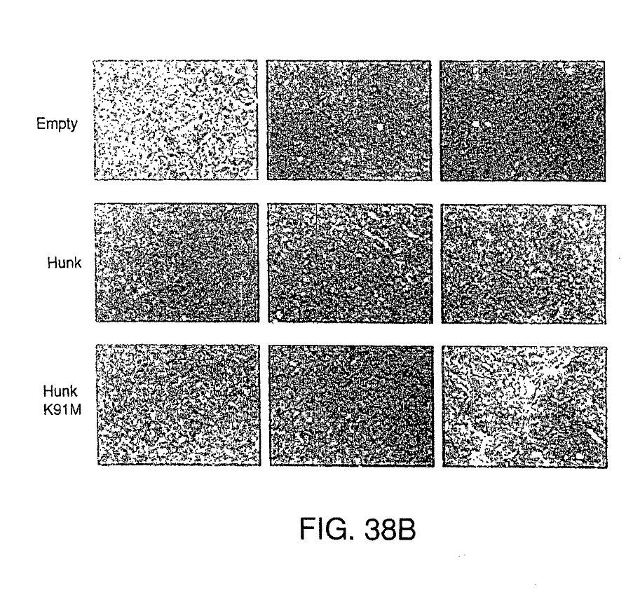

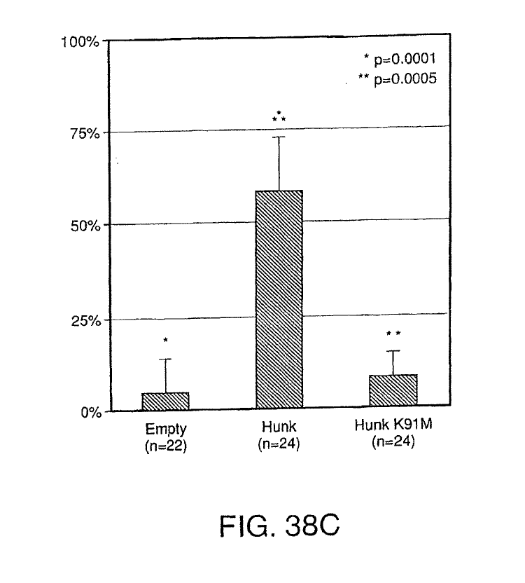

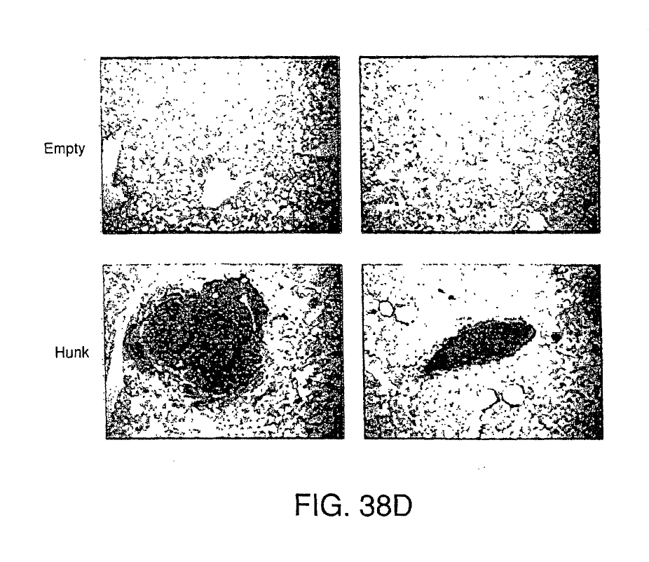

Because Hunk is able to promote migration and invasion in vitro, it was investigated whether Hunk may also be able to promote metastasis in vivo. Stably-transduced cell lines described elsewhere herein were orthotopically transplanted into the fat pads of nude mice. Mice were monitored for tumor growth and sacrificed upon reaching a mean tumor cross-sectional area of 225 mm². No differences in tumor growth were observed (FIG. 38A), consistent with the results set forth herein regarding observations of primary tumors and MW1 and MK1 tumors. Likewise, histological inspection of the tumors by H&E revealed no discernable differences between cohorts (FIG. 38B). Upon inspection of the lungs, to however, animals harboring tumors derived from Hunk expressing pools displayed a ˜11.6 fold increase in incidence of metastases when compared to empty vector controls and a ˜7.3 fold increase in incidence of metastases when compared to Hunk K91M expressing controls (FIGS. 38C and 39D). These results demonstrate that Hunk is sufficient to promote mammary tumor metastasis and that this effect is dependent on the Hunk kinase activity.

### Example 9

### HUNK Signature as a Predictor for Metastasis-Free Survival

The hierarchical clustering method described elsewhere herein (FIG. 19) demonstrates that a gene expression signature associated with HUNK expression can be used to hierarchically cluster human breast cancer samples from patients and thereby predict the likelihood of a metastatic relapse. To confirm the predictive power of gene expression signatures associated with HUNK expression, an alternative computational approach was further conducted. In this method, a centroid is calculated from a set of samples wherein the centroid is composed of the subset of genes which best distinguish HUNK-high from HUNK-low tumors (determined by the difference in expression between groups and variability within groups). Independent individual samples (external data sets) are then tested for their similarity to HUNK-high and HUNK-low tumors with respect to expression of these genes. The HUNK-high and HUNK-low centroids represent the average expression of these genes within the HUNK-high and HUNK-low groups respectively.

### Calculating the Hunk Centroids.

To apply the centroid method, microarray data were first normalized by RMA. Filters were then applied such that only probe sets that were present in at least 20% of the samples and changed at least two folds across the samples were retained. For genes with multiple probe sets only the probe set with the highest medium expression was used in analyses. Expression of each gene was scaled by the mean across samples. The centroids were defined as the within-group average expressions of the top 5% genes after ranking the genes by the ratios of between-group vs. within-group sum-of-squares of the normalized and scaled signal values.

### Preprocessing the External Human Data Sets.

Genes in the van't Veer data set were filtered by retaining only those with p-values less than 0.01 and with at least 2-fold change in at least 5 samples. Genes in the Sorlie data set were retained only if they were physically present on at least 90% of the arrays. Genes in the Ma data set were retained only if their variance is in the top 40%. Genes in the Wang data set were filter by keeping those present in at least 20% of the samples and changed at least two folds across the samples. Gene expression was scaled by the mean across samples for the Sorlie, Ma and Wang data set.

### Classifying the External Human Samples.

Samples from the four external data sets were classified into three Hunk groups based on the Pearson correlation coefficient between each sample's gene expression profile and the Hunk centroids. Samples were assigned to the high Hunk group if they have correlation coefficients higher than 0.1 with the high Hunk (or Hunk WT) centroid and lower than −0.1 with the low Hunk centroid (or Hunk WT). Samples were assigned to the low Hunk group if they have correlation coefficients higher than 0.1 with the low Hunk (or Hunk KO) centroid and lower than −0.1 with the high Hunk centroid (or Hunk KO). The remaining samples formed the intermediate Hunk group. The significance of the difference between the Kaplan-Meier survival curves of the Hunk groups within each data set was assessed by the log-rank test. Hazard ratios between the high Hunk and low Hunk groups and their significance were estimated and tested by the Cox proportional hazard model.

### Derivation and Prognostic Power of a Mouse Hunk Centroid.

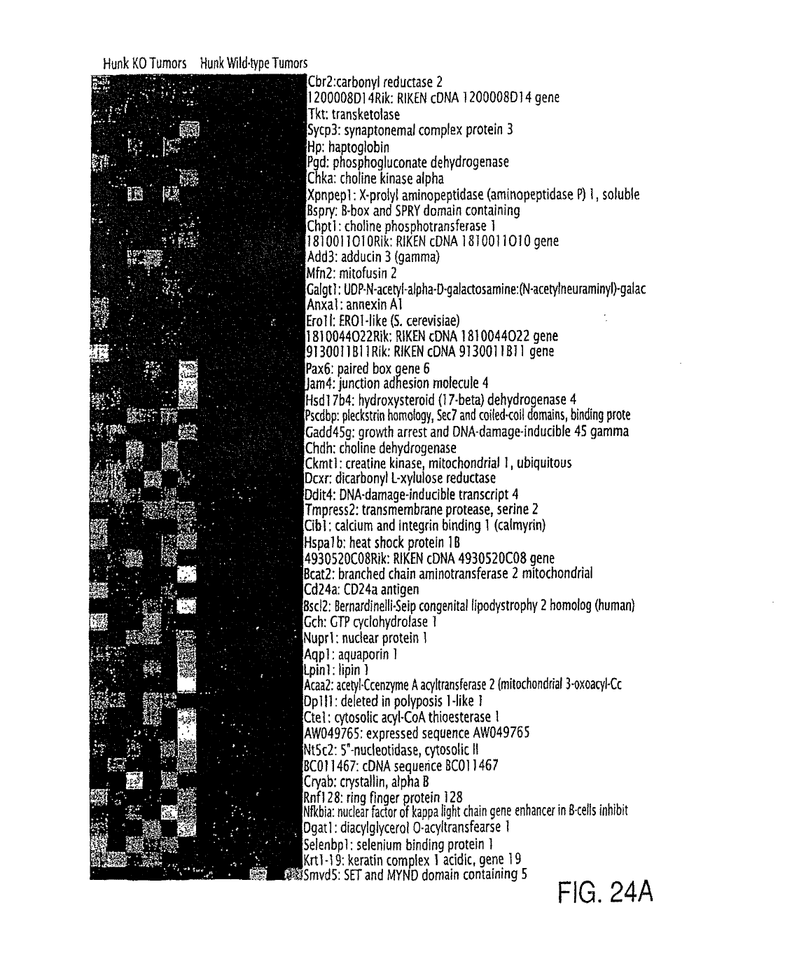

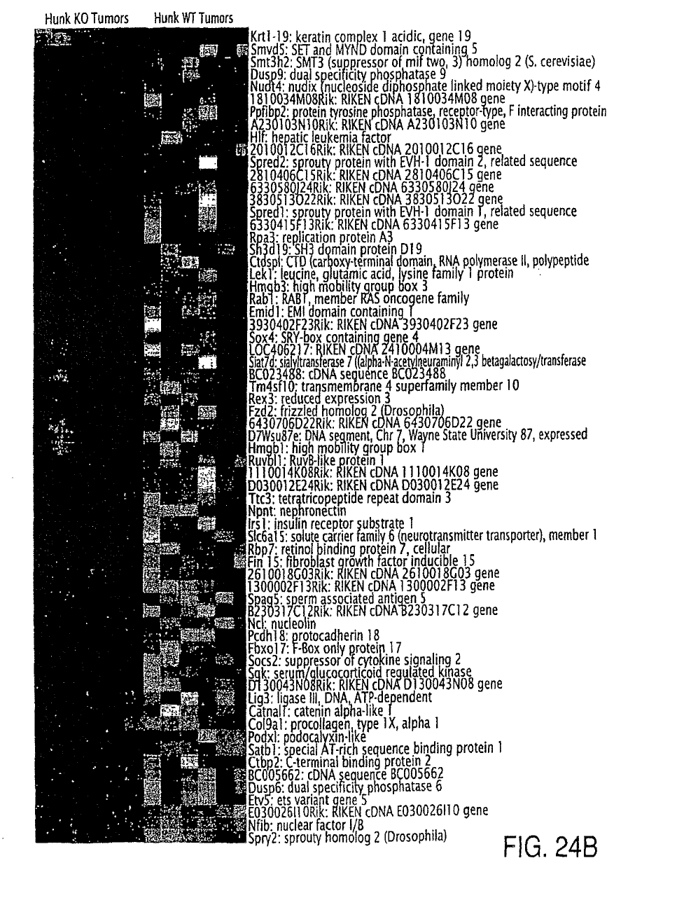

The centroid method was first applied to a set of 12 mammary tumors arising in MMTV-myc mice that were either wild-type for Hunk (6 samples) or deleted for Hunk (6 samples). Using the above methodology, a centroid was calculated consisting of those genes best able to distinguish Hunk wild-type from Hunk knockout myc-induced tumors (FIG. 24). As with the previous analysis using hierarchical clustering (FIG. 19), this centroid analysis clearly demonstrates that while these two groups of tumors are morphologically similar, they can be easily distinguished at the molecular level by gene expression profiling.

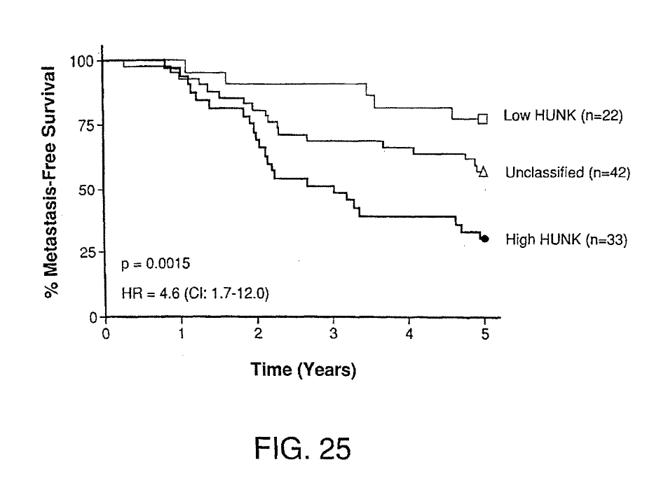

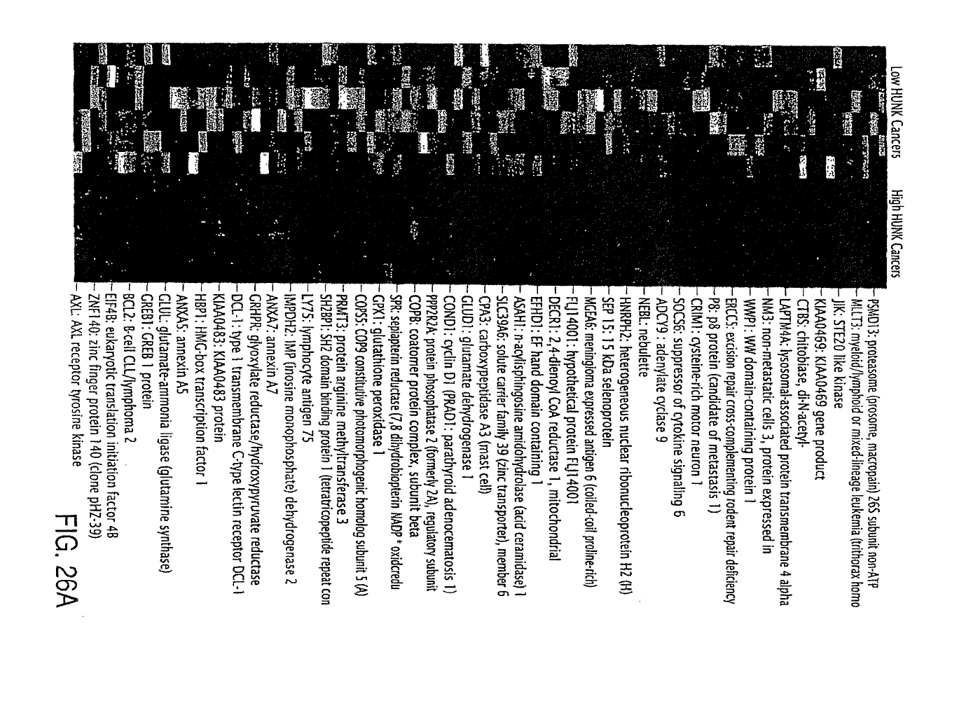

Next, the above methods were used to determine whether similarity to this mouse Hunk centroid would predict metastasis-free survival in women with breast cancer. The mouse Hunk centroid was used to classify human breast cancer samples from the van't Veer data set into those similar to Hunk wild-type tumors (High Hunk), those similar to Hunk knockout tumors (Low Hunk), and those in an intermediate group (Unclassified). Kaplan-Meier metastasis-free survival curves were then generated for each of these three groups (FIG. 25). This analysis demonstrated that women whose breast cancers were similar to the gene expression signature derived from mouse mammary tumors induced by c-myc in Hunk wild-type mice were ˜4.6-fold more likely (p<0.0015) to relapse over a five year period than women whose breast cancers were similar to the gene expression signature derived from mouse mammary tumors induced by c-myc in Hunk knockout mice (FIG. 25). Unclassified tumors showed an intermediate rate of relapse (FIG. 25). This demonstrates that similarity to a mouse Hunk centroid gene expression signature predicts decreased metastasis-free survival in women with breast cancer.

### Derivation and Prognostic Power of a Human HUNK Centroid.

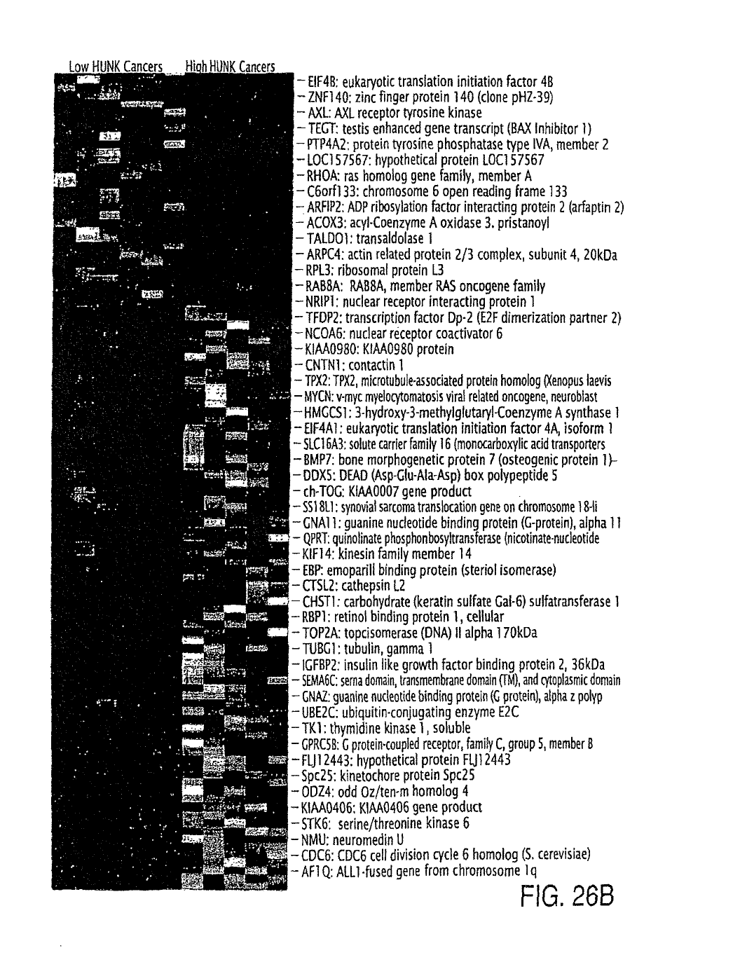

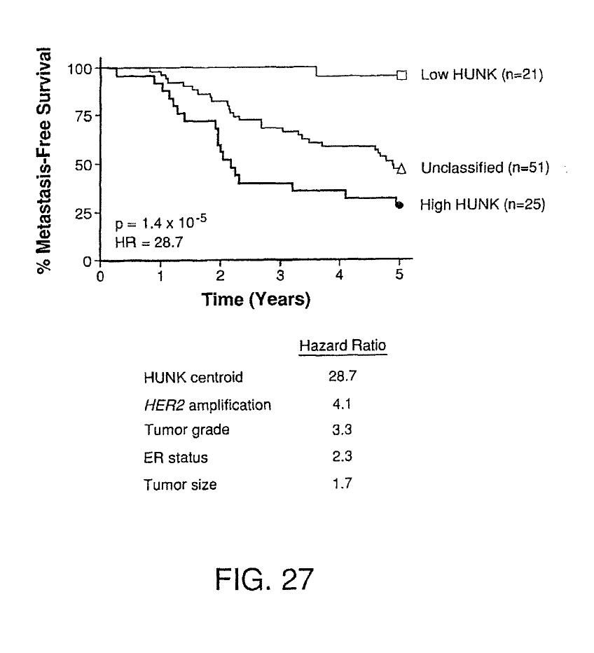

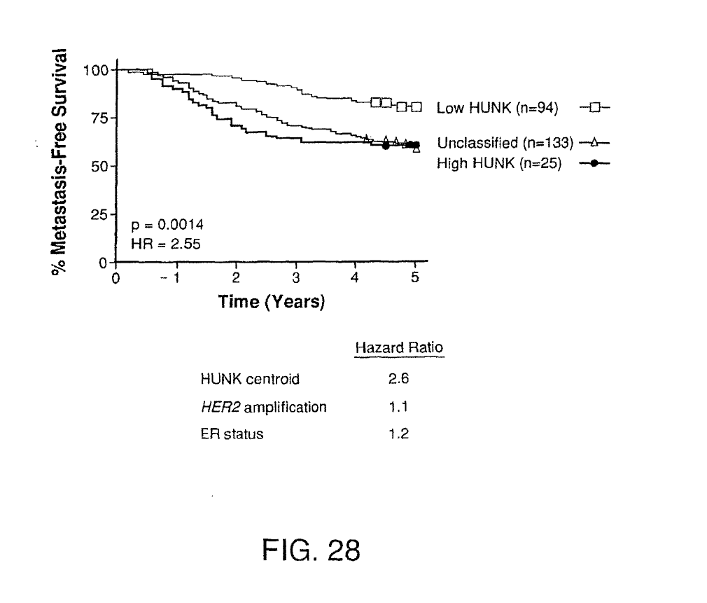

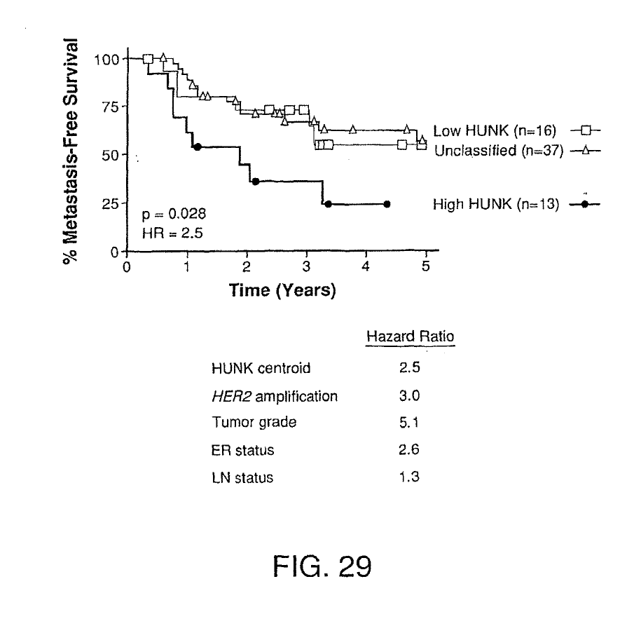

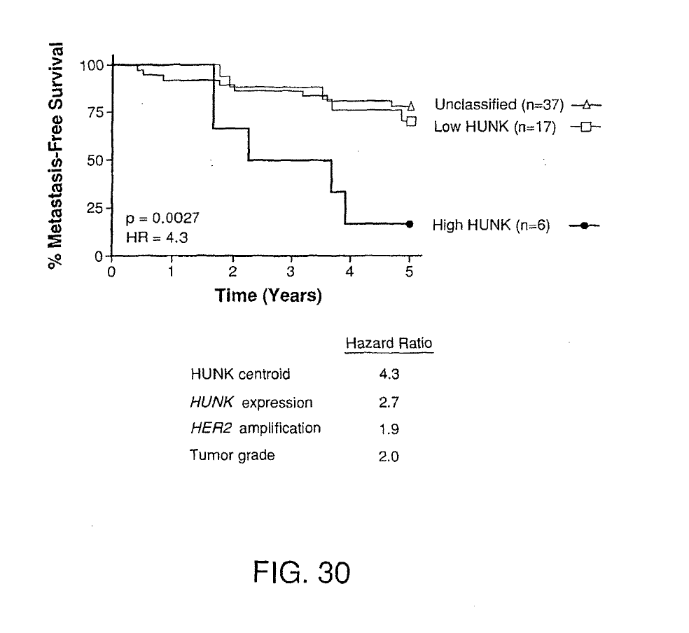

To extend these findings, it was investigated whether a similar centroid approach could be used to derive a human centroid associated with high HUNK expression in human breast cancers, and whether such a centroid was capable of predicting metastasis-free survival in human breast cancer patients. Using methods similar to those above, microarray expression profiles from human breast cancers expressing either high levels of HUNK or low levels of HUNK (FIG. 16) were used to calculate a human HUNK centroid (FIG. 26). This human HUNK centroid was then used to classify human breast cancer samples from the van't Veer, Wang, Sorlie, and Ma data sets into those most similar to high HUNK-expressing (High HUNK), low HUNK-expressing (Low HUNK), or intermediate (unclassified) breast cancers. Kaplan-Meier metastasis-free survival curves were then generated for each of these three groups for the van't Veer (FIG. 27), Wang (FIG. 28), Sorlie (FIG. 29), or Ma (FIG. 30) data sets. This analysis demonstrated that in all four data sets, women whose breast cancers were similar to the gene expression signature derived from high HUNK-expressing breast cancers were significantly more likely to relapse over a five year period than women whose breast cancers were similar to the gene expression signature derived from low HUNK-expressing breast cancers. Specifically, in the van't Veer data set, similarity to the high HUNK centroid was associated with a 28.7-fold increase in the likelihood of relapse within a five-year period (p=0.000014) (FIG. 27). This risk is far greater than that associated with high tumor grade, large tumor size, ER-negative status or HER2/neu amplification. In the Wang data set, similarity to the high HUNK centroid was associated with a 2.6-fold increase in the likelihood of relapse within a five-year period (p=0.0014), and this risk was again greater than that associated with ER-negative status or HER2/neu amplification in this patient population (FIG. 28). Similarly, in the Sorlie and Ma data sets, similarity to the high HUNK centroid was associated with a 2.5-fold (p=0.028) or 4.3-fold (p=0.0027) increase, respectively, in the likelihood of relapse within a five-year period (FIGS. 29 and 30). Finally, it is important to note that since these data sets represent a wide variety of different clinical contexts (e.g. early as well as late stage, node-negative as well as node-positive, ER-negative as well as ER-positive, and HER2/neu-amplified as well as HER2/neu-unamplified breast cancers), these findings indicate that the human HUNK centroid is a robust predictor of metastasis-free survival in women with breast cancer. In aggregate, the above centroid analysis—both mouse and human—confirm findings set forth in detail elsewhere herein using hierarchical clustering demonstrating that gene expression signatures associated with HUNK expression are powerful predictors of metastasis-free survival in women with breast cancer.

Each and every patent, patent application and publication that is cited in the foregoing specification is herein incorporated by reference in its entirety.

While the foregoing specification has been described with regard to certain preferred embodiments, and many details have been set forth for the purpose of illustration, it will be apparent to those skilled in the art that the invention may be subject to various modifications and additional embodiments, and that certain of the details described herein can be varied considerably without departing from the spirit and scope of the invention. Such modifications, equivalent variations and additional embodiments are also intended to fall within the scope of the appended claims.

What is claimed is:

1. A method of predicting metastasis-free survival of a patient diagnosed with a tumor, cancer, carcinoma, sarcoma, neoplasm, leukemia, lymphoma or hyperproliferative cell disease or oncogene expression, the method comprising detecting gene expression signature associated with elevated expression of Snf-1-related protein kinase HUNK.

2. The method of claim 1, wherein the patient is diagnosed with breast cancer.

3. A method of predicting appropriate therapy for a patient diagnosed with a tumor, cancer, carcinoma, sarcoma, neoplasm, leukemia, lymphoma or hyperproliferative cell disease or oncogene expression, wherein the method comprises predicting metastasis-free survival of said patient as in claim 1, and determining the appropriate course of therapy based on the expected metastasis-free survival of said patient.

4. The method of claim 3, wherein the patient is diagnosed with breast cancer.

5. A method of using Hunk, which encodes an Snf-1-related protein kinase, as a prognostic tool in a patient, wherein the method comprises detecting gene expression signature associated with expression of Hunk in the patient to predict the behavior of a tumor, cancer, carcinoma, sarcoma, neoplasm, leukemia, lymphoma or hyperproliferative cell disease or oncogene expression in the patient, and applying that detection to predict the appropriate therapy for the patient to treat the tumor, cancer, carcinoma, sarcoma, neoplasm, leukemia, lymphoma or hyperproliferative cell disease or oncogene expression.

6. A method of predicting an increased rate of relapse for a patient diagnosed and treated for a tumor, cancer, carcinoma, sarcoma, neoplasm, leukemia, lymphoma or hyperproliferative cell disease or oncogene expression, the method comprising detecting gene expression signature associated with elevated expression of Snf-1-related protein kinase HUNK.

7. The method of claim 6, wherein the patient is diagnosed with breast cancer.

8. A method of predicting appropriate therapy for a patient diagnosed with a tumor, cancer, carcinoma, sarcoma, neoplasm, leukemia, lymphoma or hyperproliferative cell disease or oncogene expression, wherein the method comprises predicting an increased rate of relapse for said patient as in claim 6, and determining the appropriate course of therapy based on the expected rate of relapse for said patient.

9. The method of claim 8, wherein the patient is diagnosed with breast cancer.

10. A method of using Hunk, which encodes an Snf-1-related protein kinase, as a tool to predict an increased rate of relapse in a patient, wherein the method comprises detecting gene expression signature associated with expression of Hunk in the patient to predict the behavior of a tumor, cancer, carcinoma, sarcoma, neoplasm, leukemia, lymphoma or hyperproliferative cell disease or oncogene expression in the patient, and applying that measurement to predict the appropriate therapy for the patient.

11. The method of claim 10, wherein the patient is diagnosed with breast cancer.

12. A method of diagnosing a cancer, carcinoma, sarcoma, neoplasm, leukemia, lymphoma or hyperproliferative cell disease or oncogene expression in a patient, wherein the method comprises detecting gene expression signature associated with elevated expression of Snf-1-related protein kinase HUNK.

13. The method of claim 12, wherein the patient is suspected of having breast cancer.

14. A method of using Hunk, which encodes an Snf-1-related protein kinase, as a diagnostic tool for the presence of a tumor, cancer, carcinoma, sarcoma, neoplasm, leukemia, lymphoma or hyperproliferative cell disease or oncogene expression in a patient, wherein the method comprises detecting gene expression signature associated with expression of Hunk in the patient as a molecular marker to predict the presence of a tumor, cancer, carcinoma, sarcoma, neoplasm, leukemia, lymphoma or hyperproliferative cell disease or oncogene expression in the patient.

15. A method of treating cancer, hyperproliferative disease or oncogene expression in a patient, wherein the method comprises delivering to a target cell in the patient a therapeutically effective amount of an inhibitor of Hunk to block the activation of, or decrease the activity of, HUNK in the target cell.

16. A method of treating cancer, hyperproliferative disease or oncogene expression in a patient, wherein the method comprises obtaining from the patient a biological sample, for the purpose of (1) diagnosing the presence of a HUNK-related cancer, hyperproliferative disease or oncogene expression, and (2) determining a therapeutically effective amount of an inhibitor of Hunk to administer to the patient in order to block the activation of, or decrease the activity of, HUNK in the target cell.

17. A method of treating a patient according to claim 15, wherein the inhibitor comprises an antisense or anti-Hunk molecule.

18. A method of treating cancer of claim 15, wherein the inhibitor comprises an interfering RNA.

19. The method of treating cancer as set forth in claim 15, wherein the inhibitor is a protein kinase inhibitor.

20. A method of identifying a compound that inhibits Hunk activity, wherein the effect of a compound on Hunk activity is evaluated by comparing (1) the result of contacting a cell comprising Hunk expression with said compound, with (2) the result of contacting a cell lacking Hunk expression with said compound, said method comprising the steps of:
providing a first cell comprising Hunk, then performing the steps of:
a) measuring the metastatic activity of said first cell under defined culture conditions to obtain a first metastatic value;
b) contacting said first cell with said compound;
c) measuring the metastatic activity of said first cell under said culture conditions to obtain a second metastatic value; and
d) determining the difference between said first and said second metastatic values to obtain a first inhibitory value;
providing a second, otherwise identical cell that does not comprise Hunk, then performing the steps of:
a) measuring the metastatic activity of said second cell under said culture conditions to obtain a third metastatic value;
b) contacting said second cell with said compound;
c) measuring the metastatic activity of said second cell under said culture conditions to obtain a fourth metastatic value; and
d) determining the difference between said third and said fourth metastatic values to obtain a second inhibitory value; and
evaluating the activity of said compound, wherein a first inhibitory value greater than a corresponding second inhibitory value indicates that said compound inhibits Hunk activity.


SEQUENCE LISTING (24 sequences)

SEQ ID NO: 1 - DNA, 5024 bp, Murinae gen. sp.
gcaggaggagccagggcagccccgggagccggaggaggagcggctgcgagcgcgggagccgagcgagcgcgatgccggcagcggcgggggacgggctcttgggcgagccggcggcaccggggggcgatggaggcgcggaggacacgaccaggccggcggcggcctgcgagggaagtttcctgcccgcctgggtgagcggcgtgtcccgcgagcggctccgggacttccagcaccacaagcgcgtgggcaactacctcatcggcagcaggaagctgggagagggctccttcgccaaggtgcgcgaggggctgcacgtgctgacgggagaaaaggtagctatcaaggtcatcgataagaaaagagccaagaaagacacctacgtcaccaaaaacctgcgtcgagaggggcagatccagcagatgatccgacaccccaacatcacacagctcctggacatcttggagacagagaacagctactacctggtcatggagctgtgtcctggtggcaacctcatgcacaagatctacgaaaagaaacggttggatgaagccgaggcccgcagatacatccggcaactcatctctgcggtggaacacctgcaccgtgcgggggtggttcacagagacttgaagatagagaatttgctactagatgaagacaataatatcaagctgattgactttggcttgagcaactgtgcagggatcctaggttactcggatccattcagcacacagtgtggcagccctgcctatgctgcgccagaactgcttgccaggaagaaatatggccccaaaattgatgtctggtcaataggcgtgaacatgtatgccatgctgacggggaccctacctttcactgtggagcctttcagcctgagggctctgtatcagaagatggtggacaaagcaatgaatcccctgccgacccagctctccacaggggccgtcaactttctgcgctccctcctggaaccagaccctgtgaagaggccgaatatccagcaagcgctggcgaatcgctggttgaatgagaattacactggaaaggtgccctgcaatgtcacctatcccaacaggatttctttggaagacctgagtcccagcgtggtgctgcacatgactgaaaagctgggctataagaacagtgacgtcatcaacacggtgctctccaaccgcgcctgccacatcctggccatctacttcctgttgaacaagaaacttgagcgctatttgtcagggaaatcagatatccaagatagcatctgctacaagacccagctctaccagatagagaagtgcagagccaccaaggagccctatgaggcctccctggatacctggacgagggactttgaattccatgctgtgcaggataaaaagcccaaagaacaagaaaaaagaggtgattttctccaccgtccgttttccaagaagttggacaagaacctgccttctcacaaacagccatcgccctcgctgatcacacagctccagagtaccaaagccctgctcaaagacaggaaggcctccaagtcaggcttccccgacaaagattccttcgtctgccgcaatcttttccgaaaaacctctgattccaattgtgtggcttcttcttccatggaattcatccctgtcccacctcccaggacaccaaggattgtaaagaaactagagccacaccaaccagggccgggaagtgccagcatcctccccaaggaagagcccctgctgctggatatggtacgctcctttgagtctgtggatcgagaggaccacatagaactgctgtccccttctcaccattataggatcctgagctcgcctgtgagcctggctcgtaggaattctagtgagaggacactctcccaggggctgctgtccggaagtacctcacctctccaaactccactgcattccacgctggtctcttttgcccacgaagaaaagaacagccccccgaaagaggagggtgtgtgttcaccgcctcccgttcccagtaatggcctcctgcagcctctggggagccccaactgtgtgaagagcaggggacggttccccatgatgggcatcggacagatgctgaggaagcggcaccagagcctgcagccttcctcagagaggtccctggacgccagcatgtcccctctgcagcccatagccccctccagcctctcctttgacatggccgacggtgtcaagggccagtgttaacctgggatggcaagattctgggtctctgtgaggacagccacggaacagagctccacacaggcaggcaccagggcatgggtgaacaacctcacgggagcatcctttattcttttatacctgccacacaaagtcccacgcttgtatcagctgaagtccacactcaaagtccacgcacttacttagggaccctctgagacgctgccactagggggagggggagggggcagactgtgggaatcacaccttccagcctgagattttctttgctatcaccaatcactgagccctctccaggatcccctcagtgggctcagagctaaaaaccacacctccatctgctgggccaatcagatttccagactggtaccaggttgtccctcccctcctctctgtgtgtctctcacagttctgtaactgaccgtcagtggtcagttacagtctcacgcggacgtgccactcgctggtaaggacgttcacccaacctagggatccctctacagagggaagcaaccctcctttccctaacagtgagtccccacagagtgctgagtcacagtgctggaccgggaggaagatgggatggcgcctcagacagagatggaacccagcagcgagaacccaggaggaagacgaagactcaaacgctcattcctgtgcaacgttttgacagatttttctttcctctctttctttttcccctgaccttttcttctttttgggttgaaacttgctgaggattgaacgaacttgtccaaagagatctttctttatatgaagtcattaattaaattttttttttaaagacagggtctcattaagtagcccaagctggcttcaaactcatgatcctcctgcctcagcctccaaagtgctgagattacaagtatatacccgtgtctggctcaaaatagcaattcaaaaacaaaaactagttggccagatgaaaagtagttttaccaaattcacgtgtttttgtttttctgagaggctgcagctcagatggccaaaaagctggcaacaggaggaccacagtggcctgcctgcctaagggatagtagcctagccatcctgtgtttataccgtggcagcagcagaaggcatagaacttagctccagatggctctggagagagagaaaggattcttaaagcagagttgagacagcaagaagcagggaattcgctgtgtcatgctgttctgccgtggttagaacttagctgttctgctgggagctaggagcaggcttgccgccccctgggaacacgctcacaagacggttcgtccccaaaggaaacagtgccccccaaacaggctttcagtccactctgtaatctgcaccttcccctccaggattgaaccaaagatgcatttccggttttgtgactgtgccactctgtgtgtctcttgtggaacctggtgttgtctgatcctgtccggctggcgctggatggaggactgtctctgtgtgcatcgtgggccctggtacttagcagaggacaaagggtactgttgtcaggaggggaagacttggcacgggctggaccacagttagtttagaagttatggaacagctcagaatcttctggtctttgactatttcagatggggtcagagaccagagctgtagccaggaagccaggttcatcatcttggtccatcgattctaaagtgggcaaatttctgtgacgtcacaaagccggcctttgccagtgagggctgagacacagtacaactgcctctcatttactggtggcaggcggcttcctttggcctctcagagctctgactgaactagaagagaacacggatttggctgaccctggaagaaagctgctctagtcctggctgaatttggtaagacctggactacttaaaccttagggagggactgactccctcccgaggacccattacaggaggaggccaggcttttctcccagagctgatggtgttcttcattcagcatggcttccgttcagctcccaggacttgacactgaaaatagaactctttaagcagagagaagaggagaaccatccacagacgctccccgtatttgatgtgacgtgtttgagctttgacgggtgaagagtccttttaaaagataactgccagctgcaggcatctggctctgcaaagctggtaggatgtgtacctgtgtactgtgcccgccccctttctcctagccctttatgtctttttctgactgtttgcttttctcgtatgtatgtgtgcctgtgttggtgcgagcctgtggagaaagagtctcccatccttcaaatgcttcgagaacagcgtcagatgtacaactagtttgcctgcgttgctactggtaccttggactctgaactcaggttacccacctgagtcctcagtaggcagtggacccattgagaggcaaatgagaacaggagggagacaagctgtgttctggggcgcacataaacacctgacagacgagtctaggaaaccgcgtgaaagaagaaatgttaaattctttattgttttattatatttatatggaaaatgtggctatccttttgttaagtgcagagtgtattgtctgtttgacccatgactgtccttcatgaatgagtctttgcctgtgattctagtcagcctgtggctactgatgggaacggccgatctgtcatcatgtgaagtccaggaggaagaatctattttagtcatacgatttggtcatgagtaaggactatatttatgtcaccactattgaatatatgtacttttataatggctgtgaaatacactttttcctcacaaaaaaaaaaaaaaaaaaaaaaaaaaa

SEQ ID NO: 2 - protein, 714 aa, Murinae gen. sp.
MPAAAGDGLLGEPAAPGGDGGAEDTTRPAAACEGSFLPAWVSGVSRERLRDFQHHKRVGNYLIGSRKLGEGSFAKVREGLHVLTGEKVAIKVIDKKRAKKDTYVTKNLRREGQIQQMIRHPNITQLLDILETENSYYLVMELCPGGNLMHKIYEKKRLDEAEARRYIRQLISAVEHLHRAGVVHRDLKIENLLLDEDNNIKLIDFGLSNCAGILGYSDPFSTQCGSPAYAAPELLARKKYGPKIDVWSIGVNMYAMLTGTLPFTVEPFSLRALYQKMVDKAMNPLPTQLSTGAVNFLRSLLEPDPVKRPNIQQALANRWLNENYTGKVPCNVTYPNRISLEDLSPSVVLHMTEKLGYKNSDVINTVLSNRACHILAIYFLLNKKLERYLSGKSDIQDSICYKTQLYQIEKCRATKEPYEASLDTWTRDFEFHAVQDKKPKEQEKRGDFLHRPFSKKLDKNLPSHKQPSPSLITQLQSTKALLKDRKASKSGFPDKDSFVCRNLFRKTSDSNCVASSSMEFIPVPPPRTPRIVKKLEPHQPGPGSASILPKEEPLLLDMVRSFESVDREDHIELLSPSHHYRILSSPVSLARRNSSERTLSQGLLSGSTSPLQTPLHSTLVSFAHEEKNSPPKEEGVCSPPPVPSNGLLQPLGSPNCVKSRGRFPMMGIGQMLRKRHQSLQPSSERSLDASMSPLQPIAPSSLSFDMADGVKGQC

SEQ ID NO: 3 - RNA, 10 nt, Artificial Sequence
  Kozak consensus sequence
gccrccaugg

SEQ ID NO: 4 - DNA, 6 bp, Artificial Sequence
  Mus musculus
aataaa

SEQ ID NO: 5 - DNA, 6 bp, Artificial Sequence
  Mus musculus
aataca

SEQ ID NO: 6 - protein, 6 aa, Artificial Sequence
  Mus musculus
DLKPEN

SEQ ID NO: 7 - DNA, 21 bp, Artificial Sequence
  primer
gggcccggatccacmgngayy

SEQ ID NO: 8 - DNA, 28 bp, Artificial Sequence
  primer
cccggggaattccawaggaccasacrtc

SEQ ID NO: 9 - DNA, 25 bp, Artificial Sequence
  primer
gggcccggatccrtrcacmgvgacy

SEQ ID NO: 10 - DNA, 28 bp, Artificial Sequence
  primer
cccggggaattccrwarctccasacatc

SEQ ID NO: 11 - DNA, 24 bp, Artificial Sequence
  primer
ctcactcaagattgtcagcaatgc

SEQ ID NO: 12 - DNA, 23 bp, Artificial Sequence
  primer
agggtttcttactccttggaggc

SEQ ID NO: 13 - DNA, 22 bp, Artificial Sequence
  primer
ccttaaacgcctggtgctacgc

SEQ ID NO: 14 - DNA, 26 bp, Artificial Sequence
  primer
gcagtagcctcatcatcactagatgg

SEQ ID NO: 15 - DNA, 19 bp, Artificial Sequence
  primer
ctttctttttcccctgacc

SEQ ID NO: 16 - DNA, 18 bp, Artificial Sequence
  primer
acggtgagtagcgtcacg

SEQ ID NO: 17 - protein, 714 aa, Homo sapiens
MPAAAGDGLLGEPAAPGGGGGAEDAARPAAACEGSFLPAWVSGVPRERLRDFQHHKRVGNYLIGSRKLGEGSFAKVREGLHVLTGEKVAIKVIDKKRAKKDTYVTKNLRREGQIQQMIRHPNITQLLDILETENSYYLVMELCPGGNLMHKIYEKKRLEESEARRYIRQLISAVEHLHRAGVVHRDLKIENLLLDEDNNIKLIDFGLSNCAGILGYSDPFSTQCGSPAYAAPELLARKKYGPKIDVWSIGVNMYAMLTGTLPFTVEPFSLRALYQKMVDKEMNPLPTQLSTGAISFLRSLLEPDPVKRPNIQQALANRWLNENYTGKVPCNVTYPNRISLEDLSPSVVLHMTEKLGYKNSDVINTVLSNRACHILAIYFLLNKKLERYLSGKSDIQDSLCYKTRLYQIEKYRAPKESYEASLDTWTRDLEFHAVQDKKPKEQEKRGDFLHRPFSKKLDKNLPSHKQPSGSLMTQIQNTKALLKDRKASKSSFPDKDSFGCRNIFRKTSDSNCVASSSMEFIPVPPPRTPRIVKKPEPHQPGPGSTGIPHKEDPLMLDMVRSFESVDRDDHVEVLSPSHHYRILNSPVSLARRNSSERTLSPGLPSGSMSPLHTPLHPTLVSFAHEDKNSPPKEEGLCCPPPVPSNGPMQPLGSPNCVKSRGRFPMMGIGQMLRKRHQSLQPSADRPLEASLPPLQPLAPVNLAFDMADGVKTQC

SEQ ID NO: 18 - DNA, 2826 bp, Homo sapiens
tactccagagttgttgtgtagctttgccattgaaccgatcaatttttaaactcagggcggcggcgggagaagccggggaagccgaagagcctggggaggaggagctgcgagcgcgggagacgagcaggagccgcgcgggccgcgggcgagcgcgatgccggcggcggcgggggacgggctcctgggggagccggcggcgcctgggggcggcggcggcgcggaggacgcggccaggcccgcggcggcctgcgagggaagtttcctgcctgcctgggtgagcggcgtgccccgcgagcggctccgcgacttccagcaccacaagcgcgtgggcaactacctcatcggcagcaggaagctgggcgagggctcctttgccaaggtgcgcgaggggctgcacgtgctgaccggggagaaggtggccataaaagtcattgataagaagagagccaaaaaggacacctatgtcaccaaaaacctgcggcgagagggtcagatccagcagatgatccgccaccccaatatcactcagctccttgatattttagaaacggaaaacagctactacctggtcatggagctgtgccctgggggcaacctgatgcacaagatctatgagaagaagcggctggaggagtccgaagcccgcagatacatccgacagctcatctctgccgtagagcacctgcaccgggccggggtggtccacagagacttgaagatagagaatttgctactagatgaagacaataatatcaagctgattgactttggtttgagcaactgcgcagggatcctgggttactcggatccgttcagcacacagtgtggcagccctgcctacgctgcacctgaactgctcgccaggaagaaatacggccccaaaatcgatgtctggtccataggtgtgaacatgtatgccatgttgaccgggacgctgcctttcacggtggagcctttcagcctgagggctttgtaccagaagatggtagacaaagaaatgaaccccctccccactcagctctccacaggtgccatcagtttcctgcgctctctcctggaaccggatcctgtgaagaggccaaatattcagcaggcactggcgaatcgctggcttaatgagaattacacgggcaaagtgccctgtaatgtcacctatcccaacaggatttctctggaagatctgagcccgagcgtcgtgctgcacatgaccgagaagctgggttacaagaacagcgacgtgatcaacactgtgctctccaaccgcgcctgccacatcctggccatctacttcctcttaaacaagaaactggagcgctatttgtcagggaaatctgacatccaggacagcctctgctacaagacccggctctaccagatagaaaagtacagggcccccaaggagtcctatgaggcctctctggacacctggacacgagatcttgaattccatgccgtgcaggataaaaagcccaaagaacaagaaaaaagaggggattttcttcatcgaccattctccaagaagttggacaagaacctgccctcgcacaaacagccctcaggctcgcttatgacacagattcagaacaccaaagccctcctgaaggaccggaaggcctccaagtccagcttccccgacaaagattcctttggctgccgcaatattttccgcaaaacctcagattccaattgtgtggcttcttcttccatggagttcatccccgtgccaccgcccaggaccccgaggattgtgaagaaaccggagccccatcagccagggcccggaagcactggcatcccccacaaggaagaccccctgatgctggacatggtgcgctccttcgagtctgtggatcgcgacgaccacgtagaagtgctgtctccctctcatcactacaggattctgaactccccggtcagcttggctcgcagaaattccagcgagaggacgctgtccccgggtctgccatccggaagcatgtcgcctctccatactcctttgcatccaactctggtctcttttgctcacgaagataagaacagccccccaaaagaggagggcctgtgttgcccacctccggttcccagcaatggccccatgcagcctctggggagccccaattgtgtgaaaagccgaggccggttccctatgatgggcatcggacagatgttaaggaagcgccatcagagtctgcagccatctgcagataggcccctggaggccagcctgcccccactgcagcccctagcccctgtgaaccttgcctttgacatggccgatggggtcaagacccagtgctaacttgggccagcggggtttggggtatctctagaaaacagcaactgaacagagctccacacatctgtcagggtgtgagcactccaaggcctcgcgtggagcatccttagtcccacctgtagctgaatccacagacccaaagcctgcacaacccaacctcgcttagggacccccagagatgctggaatcgctaggagggttggctccaggggcagccaattcctatcattcagatcttccttcctcccaagtactcaccaaccccttccacttcccacttcccccaggcttggggggaaaacagggcatgagccttctggggcactcagattatggactgttaccagatctttcttcacgctgtgctacatgtgtgcctctcacagcagttggccacagttacagggagagaacaatatcacagtcattcatccaggccacgtttcctctgcggagtgtagcagccctgcctttcatagcagggattacctgaaggccagcaggagccgggggcaggcccaggatcctca

SEQ ID NO: 19 - DNA, 27 bp, Artificial Sequence
  primer
ggagctgtgatgtgaagtttcctataa

SEQ ID NO: 20 - DNA, 26 bp, Artificial Sequence
  primer
aaccaggaaataactctggctcataa

SEQ ID NO: 21 - DNA, 21 bp, Artificial Sequence
  primer
caccaaagccctcctgaagga

SEQ ID NO: 22 - DNA, 25 bp, Artificial Sequence
  primer
gccacacaattggaatctgaggttt

SEQ ID NO: 23 - DNA, 24 bp, Artificial Sequence
  probe
aggccttgtgctcacccaccaaca

SEQ ID NO: 24 - DNA, 26 bp, Artificial Sequence
  probe
ctccaagtccagcttccccgacaaag